(19) United States
(12) Patent Application Publication
(10) Pub. No.: US 2024/0252520 A1
(43) Pub. Date: Aug. 1, 2024

(54) THERAPEUTIC AGENTS AND THEIR USE FOR TREATING CHRONIC WOUNDS

(71) Applicants: Beth Israel Deaconess Medical Center, Inc., Boston, MA (US); President and Fellows of Harvard College, Cambridge, MA (US)

(72) Inventors: Aristidis Veves, Westwood, MA (US); George Theocharidis, Cambridge, MA (US); Zhuqing Li, Malden, MA (US); Ioannis Vlachos, Brookline, MA (US); David J. Mooney, Sudbury, MA (US); Sangmin Lee, Cambridge, MA (US)

(21) Appl. No.: 18/408,546

(22) Filed: Jan. 9, 2024

Related U.S. Application Data
(60) Provisional application No. 63/437,996, filed on Jan. 9, 2023.

(51) Int. Cl. A61K 31/662; A61K 9/06; A61K 9/70; A61K 31/27; A61K 31/445; A61K 31/473; A61K 31/55; A61K 47/02; A61K 47/36; A61L 26/00; A61P 17/02

(52) U.S. Cl. CPC A61K 31/662; A61K 9/06; A61K 9/7023; A61K 31/27; A61K 31/445; A61K 31/473; A61K 31/55; A61K 47/02; A61K 47/36; A61L 26/0023; A61L 26/0066; A61L 26/008; A61P 17/02; A61L 2300/102; A61L 2300/216; A61L 2300/258; A61L 2300/622

(57) ABSTRACT

In certain aspects, the disclosure relates to devices and methods for treating wounds wherein an acetylcholinesterase inhibitor is delivered to a wound to promote wound healing. In further aspects, metrifonate can be delivered to the wound by time release from a wound dressing. In further aspects a sequence of wound dressings can be applied to the wound to treat different phases of wound healing.

### RELATED APPLICATIONS

This application claims priority to U.S. Provisional Patent Application No. 63/437,996 filed on Jan. 9, 2023, the contents of which are incorporated herein by reference in their entirety.

### SUBMISSION OF SEQUENCE LISTING

The Sequence Listing associated with this application is filed in electronic format and hereby incorporated by reference into the specification in its entirety. The name of the file containing the Sequence Listing is 122295_06502_Sequence_Listing. The size of the file is 23,705 bytes, and the file was created on Jan. 9, 2024.

### BACKGROUND

Impaired wound healing is an alarming problem in diabetes, attributed to the development of over 750,000 diabetic foot ulcerations (DFUs) and 70,000 lower extremity amputations per year in the US. See Gregg et al., 2014, The New England Journal of Medicine 370(16):1514-23; Armstrong et al., 2017, The New England Journal of Medicine 376(24):2367-75; and Jeffcoate et al., 2018, Diabetes Care 41(4):645-52. With the expected increase of Diabetes Mellitus (DM), DFUs will represent an even bigger burden for health systems worldwide and may prove to be one of the costliest diabetes complications. See Sen et al., 2019, Adv Wound Care 8(2):39-48. Given the severity of the problem and the rather unsatisfactory efficacy of the currently available therapeutic approaches, new, more effective treatments are urgently needed.

### SUMMARY OF THE INVENTION

In certain aspects, the disclosure relates to a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 and interleukin-17A (IL-17A), wherein the recombinant RNA molecule comprises at least one chemical modification. In some embodiments, the chemical modification is a modified uridine. In some embodiments, the modified uridine is selected from the group consisting of pseudouridine (ψ), N1-methylpseudouridine (m1ψ), N1-ethylpseudouridine, 2-thiouridine, 4′-thiouridine, 5-methylcytidine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the modified uridine is pseudouridine (ψ). In some embodiments, the chemical modification is a modified cytidine. In some embodiments, the modified cytidine is selected from the group consisting of N4-acetyl-cytidine (ac4C), 5-methyl-cytidine, 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine. In some embodiments, the modified cytidine is 5-methyl-cytidine. In some embodiments, the mRNA molecule comprises at least one pseudouridine and at least one 5-methyl-cytidine. In some embodiments, the recombinant RNA molecule is fully modified with pseudouridine. In some embodiments, the recombinant RNA molecule is fully modified with 5-methyl-cytidine. In some embodiments, the recombinant RNA molecule comprises a 5′ untranslated region (5′ UTR). In some embodiments, the recombinant RNA molecule comprises a 3′ untranslated region (3′ UTR). In some embodiments, the recombinant RNA molecule encodes CHI3L1. In some embodiments, the recombinant RNA molecule encodes CHI3L1 and IL-2 ORF7. In some embodiments, the recombinant RNA molecule encodes CHI3L1 and IL-2. In some embodiments, the recombinant RNA molecule is a recombinant mRNA molecule.

In certain aspects, the disclosure relates to a lipid nanoparticle (LNP) comprising a recombinant RNA molecule as described herein. In some embodiments, the LNP is targeted to a fibroblast.

In certain aspects, the disclosure relates to a cell comprising a recombinant RNA molecule as described herein. In some embodiments, the cell is selected from the group consisting of a fibroblast an endothelial cell, and an immune cell. In some embodiments, the immune cell is a macrophage or a T cell. In some embodiments, the cell is a fibroblast.

In certain aspects, the disclosure relates to a pharmaceutical composition comprising: (a) a recombinant RNA molecule or LNP as described herein; and (b) a pharmaceutically acceptable carrier. In some embodiments, the pharmaceutical composition is formulated for topical administration. In some embodiments, the recombinant RNA molecule is present in a therapeutically effective amount.

In certain aspects, the disclosure relates to a wound dressing comprising a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), wherein the recombinant RNA molecule comprises at least one chemical modification. In some embodiments, the recombinant RNA molecule is fully modified with pseudouridine. In some embodiments, the recombinant RNA molecule is fully modified with 5-methyl-cytidine. In some embodiments, the recombinant RNA molecule encodes CHI3L1. In some embodiments, the recombinant RNA molecule encodes CHI3L1 and IL-2 ORF71. In some embodiments, the recombinant RNA molecule encodes CHI3L1 and IL-2. In some embodiments, the recombinant RNA molecule is a recombinant mRNA molecule. In some embodiments, the recombinant RNA molecule is comprised within a lipid nanoparticle (LNP).

In certain aspects, the disclosure relates to a pharmaceutical composition comprising: (a) two or more recombinant RNA molecules each encoding a different polypeptide, wherein each of the polypeptides is selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A); and (b) a pharmaceutically acceptable carrier. In some embodiments, the two or more recombinant RNA molecules are present in a therapeutically effective amount. In some embodiments, the pharmaceutical composition comprises a recombinant RNA molecule encoding CHI3L1 and a recombinant RNA molecule encoding IL-2 ORF7. In some embodiments, the pharmaceutical composition comprises a recombinant RNA molecule encoding CHI3L1 and a recombinant RNA molecule encoding IL-2. In some embodiments, one or more of the recombinant RNA molecules comprises at least one chemical modification. In some embodiments, each of the recombinant RNA molecules is comprised within a lipid nanoparticle (LNP). In some embodiments, the pharmaceutical composition is formulated for topical administration.

In certain aspects, the disclosure relates to a wound dressing comprising a pharmaceutical composition as described herein. In some embodiments, the wound dressing comprises an alginate hydrogel. In some embodiments, the alginate hydrogel is configured to release a therapeutic dose of the recombinant RNA molecule(s) to a wound for a delivery period.

In certain aspects, the disclosure relates to a method of preparing a wound dressing, the method comprising providing a composition comprising a recombinant RNA molecule, LNP, or pharmaceutical composition as described herein, molding said composition into a desired shape, and lyophilizing said molded composition to yield a wound dressing.

In certain aspects, the disclosure relates to a method of preparing a wound dressing, the method comprising providing an alginate solution comprising a recombinant RNA molecule, LNP, or pharmaceutical composition as described herein, molding said alginate solution into a desired shape, inducing a cryo-organized structure by lyophilizing said molded alginate solution, and contacting said cryo-organized structure with a crosslinking agent to yield a wound dressing.

In certain aspects, the disclosure relates to a method of increasing expression of one or more polypeptides in a wound in a subject, wherein the one or more polypeptides is selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), the method comprising administering a recombinant RNA molecule encoding the one or more polypeptides to a wound of a subject in an amount sufficient to increase expression of the one or more polypeptides.

In certain aspects, the disclosure relates to a method of treating a wound in a subject in need thereof, the method comprising administering a therapeutically effective amount of a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A) to the wound, thereby treating the wound in the subject.

In certain aspects, the disclosure relates to a method of treating a wound in a subject in need thereof, the method comprising sequentially applying a therapeutically effective amount of a first recombinant RNA molecule and a second recombinant RNA molecule to the wound of the subject, wherein the first recombinant RNA molecule and the second recombinant RNA molecule each encode one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), and wherein the first recombinant RNA molecule and the second recombinant RNA molecule encode different polypeptides, thereby treating the wound in the subject.

In some embodiments, the wound is a diabetic wound. In some embodiments, the wound is an ulcerative wound. In some embodiments, the wound is a chronic wound. In some embodiments, the wound is a diabetic foot ulceration. In some embodiments, the wound is an acute wound. In some embodiments, the recombinant RNA molecule comprises at least one chemical modification. In some embodiments, the recombinant RNA molecule is fully modified with pseudouridine. In some embodiments, the recombinant RNA molecule is fully modified with 5-methyl-cytidine. In some embodiments, the recombinant RNA molecule encodes CHI3L1. In some embodiments, the recombinant RNA molecule encodes CHI3L1 and IL-2 ORF7. In some embodiments, the recombinant RNA molecule encodes CHI3L1 and IL-2. In some embodiments, the recombinant RNA molecule is comprised within a lipid nanoparticle.

In certain aspects, the disclosure relates to a method of treating a wound in a subject in need thereof, the method comprising administering a cell as described herein to the wound, thereby treating the wound in the subject. In some embodiments, the recombinant RNA molecule is comprised within a wound dressing. In some embodiments, the wound dressing comprises an alginate hydrogel. In some embodiments, the alginate hydrogel is configured to release a therapeutic dose of the recombinant RNA molecule to the wound for a delivery period. In some embodiments, the diabetic wound is a diabetic ulcer wound. In some embodiments, the subject is a human. In some embodiments, the recombinant RNA molecule or cell is administered topically to the site of the wound.

In certain aspects, the disclosure relates to a method of treating a diabetic ulcer wound in a subject in need thereof, the method comprising applying a wound dressing to the diabetic ulcer wound, wherein the wound dressing comprises a therapeutically effective amount of a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), and wherein the recombinant RNA molecule is comprised within a lipid nanoparticle (LNP), thereby treating the diabetic ulcer wound.

In certain aspects, the disclosure relates to a wound dressing for treating a diabetic ulcer wound, comprising an alginate hydrogel comprising a therapeutically effective amount of a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), wherein the recombinant RNA molecule is comprised within a lipid nanoparticle (LNP), and wherein the recombinant RNA molecule is distributed throughout the alginate hydrogel for release onto a diabetic ulcer wound during a delivery period.

In certain aspects, the disclosure relates to a method of identifying an mRNA associated with wound healing, the method comprising a) determining the mRNA expression profile of a first cell obtained from a wound that failed to heal; b) determining the mRNA expression profile of a second cell obtained from a wound that healed; and c) comparing the mRNA expression profile of the first cell and the second cell, thereby identifying an mRNA associated with wound healing.

In some embodiments, the method further comprises determining the mRNA expression profile of a third cell obtained from a subject that does not have a wound, and comparing the mRNA expression profiles of the first cell, second cell and third cell. In some embodiments, the mRNA expression profile is determined by single-cell RNA sequencing (scRNASeq). In some embodiments, the cell is a fibroblast. In some embodiments, the wound is a diabetic wound. In some embodiments, the wound is an ulcerative wound. In some embodiments, the wound is a chronic wound. In some embodiments, wound is a diabetic foot ulceration. In some embodiments, the wound is an acute wound.

In certain aspects, this analysis has indicated that acetylcholinesterase inhibitors such as metrifonate are suitable for the treatment of DFU wounds. Metrifonate can be incorporated into a wound dressing using an alginate hydrogel. This wound dressing can be configured for time release during the inflammatory and proliferation phases of wound healing. In one implementation, the alginate hydrogel can comprise an agent that inhibits release of the metrifonate over a selected period of time. This agent can comprise laponite that electrostatically inhibits release from the wound dressing. The metrifonate can also be incorporated into polymer microspheres that degrade over time to release from the wound dressing over a selected time period.

In further examples of treatment, wound dressings using acetylcholinesterase inhibitors can be used to treat the inflammatory phase of wounds and a further wound dressings can be used to treat to the later stages of wound healing. As these wounds generate exudate during the inflammatory phase, removal and replacement of dressings is frequently necessary. The second dressing to be applied to the wound can comprise recombinant RNA molecules as described herein that are effective to treat the later stages of wound healing. Thus, a wound dressing configured to deliver a therapeutic dose of metrifonate during a delivery period over the first 1-5 days of treatment, for example, and after removal of the first dressing, a second dressing can be applied to deliver a therapeutic dose of a second therapeutic can be applied to the wound. Additional dressings may be applied until re-epithelialization of the skin fully closes the wound.

### BRIEF DESCRIPTION OF THE FIGURES

FIG. 1 shows the phases of acute, non-complicated wound healing are shown in solid lines and the diabetic foot ulceration (DFU) healing in dotted lines. DFU is characterized mainly by the presence of chronic inflammation and, although certain wound areas may progress to the next phase, linear progression to the next phase is absent.

FIG. 2 shows t-SNE analysis depicting 14 sub-clusters of fibroblasts. The sub-clusters enriched in DFU-Healers are marked with lasso.

FIG. 3 shows UMAP of scRNASeq data of fibroblasts from DFU-Healers and DFU-non-Healer colored by their G2M score.

FIGS. 4A-4D show RT-qPCR eGFP gene expression analysis in various cell types. Representative images of cells treated with LNP-eGFP mRNA at 24h. Cells were collected at 4h, 8h, 12h, 48h, and 72h (A&B). eGFP gene expression analysis (C&D). mean±sd, n=3.

FIG. 5A shows a schematic illustration of the overall process of the mRNA-LNP alginate hydrogel bandage including molding, freeze-drying, crosslinking with calcium chloride solution, bPEI entanglement, and mRNA-LNP loading right before usage. FIG. 5B shows photographs of alginate hydrogel bandages treated with different concentration of branched polyethylene imine (bPEI); 0, 0.695, 1.39, 13.9 w/v % (top) and the amount of adsorbed mRNA measured from different bPEI concentration treated alginate bandages (bottom). The amount of initial mRNA is 4.3 ug. FIG. 5C shows cumulative Chi3Li mRNA-LNP release from alginate bandage (black) and 0.695 w/v % bPEI entangled alginate bandages (navy). FIG. 5D shows 3t3 cell viability from different bPEI concentration treated alginate bandages. mean±sd

FIGS. 6A-6C show application of LNP-mRNA bandages to diabetic mice wounds. (A) Representative images of wounds on day 0 (D0) and day 10 (D10) for blank bandage, LNP-fibroblast growth factor 2 (FGF-2) mRNA bandage, LNP-interleukin 2 ORF7 (IL-2 ORF7) mRNA bandage, LNP-CHI3L1 mRNA bandage, and LNP-IL-17A mRNA bandage. (B) The wound healing curve for all groups from D0 to D10. (C) Quantification of the wound closure on D10. mean±sd, n=10-14. IL-2 in the figure indicates IL-2 ORF7.

FIG. 7A shows RT-qPCR analysis of delivered CHI3L1 mRNA in wounds treated with LNP-CHI3L1 mRNA or blank (BLK) bandages on days 1, 3, 5, 7, and 10. FIG. 7B shows Western blot analysis on protein expression at Day 10. FIG. 7C shows RT-qPCR analysis of mouse gene expression for Col1a1, Fn1, Tgfb1, Vegfa, and Il-10 fold change on D6 compared to D3. mean±sd, n=3. IL-2 in the figure indicates IL-2 ORF7.

FIGS. 8A-8B show wound healing studies in porcine skin. Schematic of dermal wound treatment dressing allocation in the diabetic mini-swine (Yucatan's) model (A). Progression of a wound covered with Tegaderm (TD) only (B).

FIG. 9 shows the expression of prioritized markers (n=118) for the MERSCOPE panel across different cell populations (scRNAseq data).

FIGS. 10A-10D show spatial transcriptomic and proteomic profiling of human skin from DFU patients. (A) Visualization of all different probes of MERSCOPE (single molecule FISH) profiling of a human skin sample using a 140-plex targeted gene panel (in-house generated data). (B) Selected regions of the MERSCOPE slide show cellular populations are well defined. Blue: CFD, DCN (Fibroblasts), Light Green: ACTA2, TAGLN (Smooth Muscle Cells), Red: CD4, CD8 (T cells). (C) Heatmap of marker gene expression for cellular populations. (D) Human skin sample subjected to highly multiplexed (21-plex antibody panel) immunofluorescence using the CODEX assay.

FIGS. 11A and 11B show application of blank hydrogel to humanized mice dorsal wound enhanced wound healing. (A) Schematic illustrations of the development of STZ induced diabetic humanized mice. (B) Representative images of wounds on D0, D2, D5 and D7.

FIGS. 12A-12C show endothelial cells, keratinocytes, and human dermal fibroblast migration in vitro 24 hours and 72 hours after scratching. (A) Endothelial cells, quantitation of scratch wounds after 24h. (B&C) Keratinocytes and Human dermal fibroblasts quantitation of scratch wound after 72h. Data represent mean±SD of n>8 observations for three cell lines from three biologically independent experiments. IL-2 in the figure indicates IL-2 ORF7.

FIG. 13 shows the application of a bandage containing LNP-Chi3L1 and LNP-IL-2 ORF7 mRNA to a diabetic mouse dorsal wound. LNP-Chi3L1 mRNA and LNP-IL-2 ORF7 mRNA were loaded to the bandage simultaneously. The wound healing curve is shown for all groups from D0 to D10. Size of the wounds were compared to the wound size on DO. Values represent the mean±and the standard deviation (n=14-16). IL-2 in the figure indicates IL-2 ORF7.

FIG. 14A-14F shows CHI3L1 overexpression in dermal fibroblasts. (A) Western blot analysis of two constructs (1 and 2) overexpressing CHI3L1, untreated cells and control (CTRL) construct expressing cells. (B) RT-qPCR of CHI3L1 (C,D) Representative images (C) and quantitation (D) of crystal violet stained cells. (E,F) Representative images (E) and quantitation (F) of scratch wounds.

FIG. 15A-15C shows CHI3L1 overexpressing fibroblasts improve diabetic wound healing (A) Wound closure as % of open wound compared to day 0 over time. Quantitation of (B) Day 11 angiogenesis and (C) dermal cell proliferation. n=7-8 wounds, mean±sd, unpaired t-test.

FIG. 16A-16B shows M1 Mf enrichment in CHI3L1-OE Fb treated wounds (A) Representative immunofluorescence wound images on Day 11. (B) CD11b and TNFa double + cells. n=7 wounds, mean±sd, unpaired t-test.

FIGS. 17A and 17B show the effects of LNP-CHI3L1 mRNA and LNP-IL-2 ORF7 mRNA on wound healing in a humanized skin graft mouse model with streptozotocin (STZ)-induced diabetes. FIG. 17A shows the wound healing curve for LNP-CHI3L1 mRNA, LNP-IL-2 ORF7 mRNA, and LNP-eGFP mRNA (negative control) bandage-treated wounds from Day 0 to Day 7. The size of the wounds was compared to the wound size on D0. p values indicate differences between LNP-eGFP mRNA (negative control) and the two active treatments, LNP-CHI3L1 mRNA and LNP-IL-2 ORF7 mRNA. There was no difference between LNP-CHI3L1 mRNA and LNP-IL-2 ORF7 mRNA. FIG. 17B shows quantification of the wound closure on Day 7. Values represent the mean and the standard deviation (n=11-18). IL-2 in the figure indicates IL-2 ORF7.

FIG. 18 shows representative immunofluorescent images for CHI3L1 and a Vimentin/Macrophage (RM0029-11H3) marker on blank bandage and CHI3L1 mRNA treated diabetic mouse wounds on day 3.

FIGS. 19A-19C show that the application of LNP-mRNA loaded bandages to db/db mouse wounds enhanced healing. FIG. 19A shows representative images of wounds on day 0 (D0) and day 10 (D10). FIG. 19B shows the wound healing curve from Day 0 to Day 10. Using General Linear Model for repeated measurements, treatment with CHI3L1 mRNA performed better when compared to all other treatments except IL-17 mRNA (P<0.0001). FIG. 19C shows quantification of the wound closure on Day 10. Mean±sd (n=10-20). Statistical significance and P values were determined by one-way ANOVA followed by Tukey's multiple comparison test. IL-2 in the figure indicates IL-2 ORF7.

FIGS. 20A-20C show LNP-mRNA treated human skin xenografted mice wounds. FIG. 20A shows a schematic illustration for the xenotransplantation, STZ induction, and wounding procedures. FIG. 20B shows the wound healing curve for LNP-CHI3L1 mRNA and LNP-eGFP mRNA bandages treated wounds from D0 to D7. Size of the wounds was compared to the wound size on Day 0. Values in A and B represent the mean and the standard deviation (n=11-18). FIG. 20C shows representative images of wounds on day 0 and day 7. mean±s.d. (n=12-18) and P values were determined by t-test.

FIGS. 21A-21D show single-cell RNAseq analysis on CHI3L1 mRNA treated human skin xenografted mice wounds compared to eGFP mRNA treated wounds. FIG. 21 A shows a UMAP of all identified cell types. FIG. 21B shows a heatmap of the proportion of cell types per condition. FIG. 21C shows counts of CHI3L1 between conditions in each cell type. FIG. 21D shows a Pathway barplot of the Fibro_1 cluster, displaying the top 10 deregulated pathways between the CHI3L1 mRNA and eGFP mRNA treated wounds for the Biological Process category.

FIGS. 22A-22E show that the application of LNP-mRNA loaded bandages to human skin wounds enhanced healing. FIG. 22A shows schematics of standardized & ready-to-use ex vivo human skin culturing system from Genoskin®. FIG. 22B shows representative images of wounds on day 0 and day 4. FIG. 22 C shows representative Day 4 Masson's trichrome (MTS) histology images, arrow indicates the stained fibrous tissue area. FIG. 22D shows quantification of fibrous tissue area. FIG. 22E shows quantification of the wound closure expressed as % of open wound compared to Day 0,values represent the mean±s.d. (n=5 for both LNP-eGFP mRNA and LNP-CHI3L1 mRNA groups).

FIGS. 23A and 23B show protein expression and CHI3L1 levels in LNP-CHI3L1 mRNA treated pig dermal fibroblasts. FIG. 23A shows western blot analysis of protein expression from LNP-Chi3L1 mRNA (500 ng/ml) treated pig dermal fibroblasts from Day 1 to Day 5 with one dosage. FIG. 23B shows secreted CHI3L1 protein levels in CHI3L1 mRNA treated pig dermal fibroblasts on day 3.

FIG. 24A shows the 48h and 72 hour Alamar Blue reduction percentage defined metrifonate concentration (10 micrograms/ml) used for measurements.

FIG. 24B shows the quantitation of scratch wounds at 0 and 24 hours.

FIGS. 24C-E shows gene expression of selective markers in naïve and polarized macrophages.

FIG. 25A shows a method for fabricating a wound dressing with metrifonate incorporated within an alginate hydrogel (met-gel).

FIG. 25B shows three different metrifonate loading concentrations (0.08 mg/ml, 0.5 mg/ml and 2 mg/ml for invitro release study.

FIG. 25C shows the in vitro daily release of LD met-gel in weight for seven days.

FIG. 26A-C shows the application metrifonate gel applied to a diabetic mouse dorsal wound for enhanced wound healing wherein (A) shows images of wounds on day 0 (D0) and day 10 (D10) for Tegaderm® with soluble metrifonate (met-sol) applied topically, or low dose met-gel removed at D3, met-gel removed at D6, or medium dose met-gel removed at D3. (B) shows wound healing curve for all groups D0 to D10. (C) shows quantification of the wound closure on D19 with mean+/−sd (n=10-16 and p<0.05.

FIG. 27 shows the QC pipeline performed for a MERSCOPE dataset, capturing transcript abundance, density, accuracy, cell number, boundaries and nuclei detected.

FIG. 28 shows the wound healing curve for all groups from D0 to D7 with the application of metrifonate gel to humanized mice.

FIG. 29 shows metrifonate hydrogel treated humanized mice wounds having increased gene expression for IL-10 (A) and Arg(B) and gene VEGFa(C) with a mean+/−sd, n=4-8.

FIG. 30 shows an illustrative cross-section of a wound dressing such as a bandage to be applied to a chronic wound for therapies as described herein.

FIGS. 31A-31H illustrate day 5 and day 10 data that were analyzed for three treatment groups: blank gel (blk gel) and LD-Met gel (A). Compared to Blk-Gel treated wounds on day 5, the LD Met-Gel significantly accelerated wound healing with a higher re-epithelialization rate (B) and vessel density (D). On day 10, except wound closure rate (E) and HPE area (G), no significance was observed for re-epithelization, and CD31 counts (F & H).

FIGS. 32A-32F illustrate comparisons of LD met-gel treatments and blank dressings where multiple healing metrics were positively regulated including increased re-epithelialization (A) and angiogenesis (B), as well as reduced fibrosis (Tgfb1, Acta2) and inflammation (Il1b, Mmp9) markers gene expression (C). Intriguingly, metrifonate led to enrichment of both total and degranulated mast cells (E), as discerned by toluidine blue staining (D). We have previously identified these granulocytes as integral mediators of diabetic wound healing. We also validated that metrifonate treatment inhibits Acetylcholinesterase (AchE) activity by measuring the levels of the enzyme on Day 5 mouse wounds ( ), a time point that is 2 days after bandage removal

FIGS. 33A-33E illustrate modulating inflammatory response by staining for two major cell types that are involved in the early inflammatory stage, neutrophil and macrophages. Metrifonate bandage application decreases the levels of neutrophil marker NIMP-R14+ cells (A green, B) but increases the number of macrophage marker F4/80+ cells (A red, C). This corroborates the findings indicating that metrifonate promotes neutrophil clearance and facilitates monocyte to macrophage differentiation. The protein expression of both MMP-9 and NGAL (markers for neutrophils) were down regulated by LD-Met gel (D & E).

FIG. 34 illustrate an in vivo study was conducted using db/db mouse model with a day 1 and day 2 sacrifice time points where blank gel served as control, and LD-Met gel was the selected treatment.

FIGS. 35A-35F illustrate gene expression analyses of isolated neutrophils and the rest of the wound cells on day 1 and day 2, using a panel of nicotinic and muscarinic receptors (A-B, E-F). The gene expression of Chrm1 and Chrm2 for isolated neutrophil from the wounds treated by LD-Met gel on day 1 were upregulated (A), whereas only Chrm3 expression showed a 7-fold change in isolated neutrophil collected from LD-Met gel treated wounds compared to neutrophils in blk gel treated wounds on day 2 (D). Intriguingly, Chrm1, Chrm3, Chrna7 were markedly activated in the rest of the cell types by LD-Met gel on day 1 compared to blk gel, and remained continually activated by LD-Met gel through day 2 (E). To further explore the specific cell types that may involve in this process, RT-qPCR gene expression analysis was carried out for the rest of the cell collections from the wounds where specific primers representing canonical markers for different cell types were selected for both day 1 and day 2 (C & F).

FIG. 36A-36C illustrate that nicotinic and muscarinic receptors gene expressions were further studied in human primary cell lines with important roles in the wound healing process, including THP-1 monocyte derived macrophages (M0 naïve macrophages, LPS activated M1 macrophages, and IL-4 & IL-13 activated M2 macrophages), keratinocytes, and fibroblasts (A-C)

FIGS. 37A-37D demonstrate that metrifonate acts through Chrm1 (M1), Chrm3 (M3) muscarinic receptors or α7nAChR nicotinic receptor, another in vivo study was conducted using db/db mouse model with day 5 sacrifice. Highly selective agonists or antagonists were chosen for M1, M3, and α7nAChR receptors and topically applied (2 mg/kg) to the wound area daily. Notably, with M1 antagonist (VU0255035) applied, LD-Met gel treated wounds performed comparably to Blk gel treated wounds (A), whereas M3 (J104129) and α7nAChR (Methyllycaconitine citrate) antagonists had no impact on wound closure rate to both treatment groups when compared to LD-Met gel and blk gel treated wounds with no application of any antagonist. FIGS. 37B & 37C validate the function of Chrm1 receptor, we employed Chrm1 (McN-A 343), Chrm3 (LASSBIo0897), and α7nAChR (PNU-282987) agonists, molecules that can bind and functionally activate the target receptor, separately to the wound area daily with blk-gel treatment to confirm if the agonist(s) are capable of mimicking the function of metrifonate (D). Of note, solely the M1 agonist promoted the wound healing, and outperformed M3 and α7nAChR agonists.

FIG. 38 illustrate the biomaterials that were placed on acute porcine wounds over 20 days and found that metrifonate did not induce any adverse effects and promoted better healing as evidenced by shorter wound lengths in histological assessment (A & B).

FIG. 39 illustrates an exemplary method for applying a first wound dressing for delivery of an AchE material to an ulcerative wound to treat the inflammatory phase of the wound and applying a second wound dressing to treat the wound during a later phase of wound healing with an mRNA molecule.

### DETAILED DESCRIPTION

In certain aspects, the present disclosure relates to recombinant nucleic acid molecules (e.g., RNA molecules) encoding one or more proteins selected from the group consisting of chitinase-3-like protein 1 (CHI3L1), a fibroblast growth factor (FGF), interleukin-2 open reading frame 7 (IL-2 ORF7), an interleukin-17 (IL-17) protein, and an IL-17 receptor, wherein the recombinant nucleic acid molecule (e.g., RNA molecule) comprises at least one chemical modification. In certain aspects, the present disclosure also relates to a method of treating a wound (e.g., an acute, chronic and/or diabetic wound) in a subject in need thereof, the method comprising administering a therapeutically effective amount of a recombinant nucleic acid molecule (e.g., RNA molecule) encoding one or more proteins selected from the group consisting of chitinase-3-like protein 1 (CHI3L1), a fibroblast growth factor 2 (FGF), interleukin-2 ORF7 (IL-2 ORF7), an interleukin-17 (IL-17) protein, and an IL-17 receptor to the wound, thereby treating the wound in the subject. In some embodiments, the one or more proteins are selected from the group consisting of CHI3L1, FGF-2, IL-2 ORF7 and IL-17A.

Single-cell RNA-sequencing (scRNASeq) analysis provides deep insight into cell function and disease pathophysiology by allowing the profiling of the transcriptomic landscape of individual cells in heterogeneous tissues. A recently completed study in our unit primarily focused on differences between diabetic foot ulceration (DFU) patients who heal their ulcers (Healers) and DFU patients who fail to heal them (non-Healers) and investigated molecular changes via scRNASeq analysis of surgically removed DFUs. See Theocharidis et al., 2022, Nature communications 13(1):181, which is incorporated by reference herein in its entirety. Our analysis showed enrichment of a unique population of fibroblasts, named Healing Enhancing (HE)-fibroblasts, overexpressing MMP1, MMP3, MMP11, HIF1A, CHI3L1, and TNFAIP6 genes in the DFU patients with healing wounds. Pathway analysis indicated the activation of multiple immune and inflammatory pathways including IL6, HIF1A and ILK signaling in HE-fibroblasts.

Delivery of mRNA into target cells has the potential to provide functional protein expression that can lead to a broad array of applications. The experimental results provided herein demonstrate that bandages that provide controlled lipid nanoparticle (LNPs) delivery of modified CHI3L1, IL-17A, FGF-2, IL-2 and IL-2 ORF7 mRNAs considerably improve wound healing in diabetic db/db mice. Based on these findings, topical wound treatment with biomaterials that release mRNA corresponding to genes shown to promote wound healing can lead to the development of new therapeutic approaches. Furthermore, combination of various mRNAs with specific temporal delivery can physiologically imitate the activation of various pathways that are associated with DFU healing.

### I. Definitions

The terms “administer”, “administering” or “administration” include any method of delivery of a pharmaceutical composition or agent into a subject's system or to a particular region in or on a subject.

As used herein, “administering in combination”, “co-administration” or “combination therapy” is understood as administration of two or more active agents using separate formulations or a single pharmaceutical formulation, or consecutive administration in any order such that, there is a time period while both (or all) active agents overlap in exerting their biological activities. It is contemplated herein that one active agent (e.g., a first mRNA or cell) can improve the activity of a second therapeutic agent (e.g. a second mRNA or cell), for example, can sensitize target cells to the activities of the second therapeutic agent or can have a synergistic effect with the second therapeutic agent. “Administering in combination” does not require that the agents are administered at the same time, at the same frequency, or by the same route of administration.

The terms “chemical modification” and “chemically modified” refer to modification relative to a wildtype nucleic acid molecule with respect to adenosine (A), guanosine (G), uridine (U), thymidine (T) or cytidine (C) ribonucleosides or deoxyribonucleosides in at least one of their position, pattern, percent or population. In some embodiments, a chemical modification is a replacement of a nucleoside with a different form of the same nucleoside, e.g., a replacement of uridine with pseudouridine, or a replacement of cytidine with 5-methyl-cytidine. In some embodiments, a chemical modification is a replacement of a nucleoside with a different nucleoside, e.g., a replacement of uridine with cytidine. Generally, these terms do not refer to the ribonucleotide modifications in naturally occurring 5′-terminal mRNA cap moieties. With respect to a polypeptide, the term “modification” refers to a modification relative to the canonical set of 20 amino acids. Polypeptides, as provided herein, are also considered “modified” if they contain amino acid substitutions, insertions or a combination of substitutions and insertions.

As used herein, the term “circular RNA” refers to a polyribonucleotide that forms a circular structure through covalent or non-covalent bonds. Circular RNAs are described, for example, in U.S. Pat. No. 11,160,822, which is incorporated by reference herein in its entirety.

As used herein, the terms “increasing” and “decreasing” refer to modulating resulting in, respectively, greater or lesser amounts, function or activity of a parameter relative to a reference. For example, subsequent to administration of a composition described herein, a parameter (e.g., would size) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun.

The term “heterologous” as used herein refers to a combination of elements that do not naturally occur in combination. For example, a polynucleotide that is heterologous to a virus or target cell refers to a polynucleotide that does not naturally occur in the virus or target cell, or that occurs in a position in the virus or target cell that is different from the position at which it occurs in nature. A polypeptide that is heterologous to a target cell refers to a polypeptide that does not naturally occur in the target cell, or that is expressed from a polynucleotide that is heterologous to the target cell.

The terms “interleukin-2 ORF7” or “IL-2 ORF7” as used herein refer to the polypeptide encoded by open reading frame 7 of the human interleukin 2 gene (NCBI Accession No. NM_000586.4, which is incorporated by reference herein in its entirety). The amino acid sequence of IL-2 ORF7 is provided herein as SEQ ID NO: 6, and the nucleic acid sequence of open reading frame 7 of the human interleukin 2 gene is provided herein as SEQ ID NO: 5.

A “subject” to be treated by the methods of the invention can mean either a human or non-human animal, e.g., a mammal. In some embodiments, the subject is a human. In some embodiments, the subject is a non-human mammal. In some embodiments, the non-human mammal is a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In certain embodiments, a subject has a wound (e.g., a chronic wound, an acute wound, an ulcerative wound, or a diabetic wound such as a diabetic foot ulceration) prior to initiation of treatments using the methods of the invention. In certain embodiments, the subject has diabetes prior to initiation of treatments using the methods of the invention.

The term “recombinant nucleic acid molecule” as used herein refers to a nucleic acid molecule that comprises a combination of two or more polynucleotides that form a nucleic acid molecule that is not found in nature. Accordingly, a recombinant nucleic acid molecule comprises at least two polynucleotides that are covalently bound to a nucleic acid sequence to which they are not covalently bound in nature. For example, in some embodiments, a recombinant nucleic acid molecule comprises two or more polynucleotides, each encoding a different therapeutic polypeptide (e.g., CHI3L1, FGF-2, IL-2 ORF7, IL-2 or IL-17), wherein the two or more polynucleotides are covalently bound to a nucleic acid sequence to which they are not covalently bound in nature (e.g., covalently bound to each other). In some embodiments, the recombinant nucleic acid molecule comprises at least two polynucleotides that are not found within the same nucleic acid molecule in nature.

For example, the term “recombinant RNA molecule” as used herein refers to an RNA molecule (e.g., an mRNA molecule) that comprises a combination of two or more polyribonucleotides that form an RNA molecule that is not found in nature. Accordingly, a recombinant RNA molecule comprises at least two polyribonucleotides that are covalently bound to a polyribonucleotide sequence to which they are not covalently bound in nature (e.g., covalently bound to each other). Specifically, in some embodiments, a recombinant RNA molecule comprises two or more polyribonucleotides, each encoding a different therapeutic polypeptide (e.g., CHI3L1, FGF-2, IL-2 ORF7, IL-2 or IL-17), wherein the two or more polyribonucleotides are covalently bound to a polyribonucleotide to which they are not covalently bound in nature (e.g., covalently bound to each other). In some embodiments, the recombinant RNA molecule comprises at least two polyribonucleotides that are not found within the same RNA molecule in nature.

The term “recombinant DNA molecule” as used herein refers to a DNA molecule that comprises a combination of two or more polydeoxyribonucleotides that form a DNA molecule that is not found in nature. Accordingly, a recombinant DNA molecule comprises at least two polydeoxyribonucleotides that are covalently bound to a polydeoxyribonucleotide sequence to which they are not covalently bound in nature (e.g., covalently bound to each other). For example, in some embodiments, a recombinant DNA molecule comprises two or more polydeoxyribonucleotides, each encoding a different therapeutic polypeptide (e.g., CHI3L1, FGF-2, IL-2 ORF7, IL-2 or IL-17), wherein the two or more polydeoxyribonucleotides are covalently bound to a polydeoxyribonucleotide to which they are not covalently bound in nature (e.g., covalently bound to each other). In some embodiments, the recombinant DNA molecule comprises at least two polydeoxyribonucleotides that are not found within the same DNA molecule in nature.

“Therapeutically effective amount” means the amount of a compound that, when administered to a patient for treating a disease or condition (e.g., a wound), is sufficient to effect such treatment for the disease or condition. When administered for preventing a disease or condition (e.g., a wound), the amount is sufficient to avoid or delay onset of the disease or condition (e.g., wound formation). The “therapeutically effective amount” will vary depending on the compound, the disease or condition and its severity, and the age, weight, etc., of the patient to be treated. A therapeutically effective amount need not be curative. A therapeutically effective amount need not prevent a disease or condition from ever occurring. Instead, a therapeutically effective amount is an amount that will at least delay or reduce the onset, severity, or progression of a disease or condition. In some embodiments, the therapeutically effective amount is the amount effective to treat a subject having a disease or condition (e.g., a wound) at the time of administration, i.e., to treat an existing disease or condition (e.g., a wound).

The term “therapeutic polypeptide” or “therapeutic protein” as used herein refers to a polypeptide selected from the group consisting of chitinase-3-like protein 1 (CHI3L1), a fibroblast growth factor (FGF), interleukin-2 ORF7 (IL-2 ORF7), interleukin-2 (IL-2) an interleukin-17 (IL-17) protein, and an IL-17 receptor. In some embodiments, the FGF is FGF-2. In some embodiments, the IL-17 protein is IL-17A. In some embodiments, the IL-17 receptor is IL-17RE. In some embodiments, the therapeutic protein is selected from the group consisting of CHI3L1, FGF-2, IL-2 ORF7, IL-2 and IL-17A.

As used herein, “treatment”, “treating” and cognates thereof refer to the medical management of a subject with the intent to improve, ameliorate, stabilize, prevent or cure a disease, condition (e.g., a wound), or disorder. This term includes active treatment (treatment directed to improve the disease, condition, or disorder), causal treatment (treatment directed to the cause of the associated disease, condition, or disorder), palliative treatment (treatment designed for the relief of symptoms), preventative treatment (treatment directed to minimizing or partially or completely inhibiting the development of the associated disease, condition, or disorder); and supportive treatment (treatment employed to supplement another therapy).

The term “variant” as used herein with reference to a polypeptide refers to a polypeptide that differs by at least one amino acid residue from a corresponding wild type polypeptide. In some embodiments, the variant polypeptide has at least one activity that differs from the corresponding naturally occurring polypeptide. The term “variant” as used herein with reference to a polynucleotide refers to a polynucleotide that differs by at least one nucleotide from a corresponding wild type polynucleotide. In some embodiments, a variant polypeptide or variant polynucleotide has at least 50%, 55%, 60%, 65%, 70%, 75%, 80%, 85%, 90%, 95%, 96%, 97%, 98%, or 99% sequence identity to the corresponding wild type polypeptide or polynucleotide and differs by at least one amino acid residue. In some embodiments, the variant is a functional fragment of a polypeptide.

A “5′ untranslated region” (5′UTR) as used herein refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

An “open reading frame” (ORF) is a continuous stretch of DNA or RNA beginning with a start codon (e.g., methionine (ATG)), and ending with a stop codon (e.g., TAA, TAG or TGA) and encoding a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

### II. Nucleic Acid Molecules

In certain aspects, the disclosure relates to a nucleic acid molecule (e.g., a recombinant nucleic acid molecule) encoding one or more therapeutic proteins. The term “therapeutic protein” as used herein refers to a protein selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL-17 receptor. In some embodiments, the FGF is FGF-2. In some embodiments, the IL-17 protein is IL-17A. In some embodiments, the IL-17 receptor is IL-17RE. In some embodiments, the therapeutic protein is selected from the group consisting of CHI3L1, FGF-2, IL-2 ORF7, IL-2 and IL-17A.

In some embodiments, at least one of the therapeutic proteins is CHI3L1. CHI3L1 belongs to glycoside hydrolase family 18, which includes chitinases and non-enzymatic chitinase-like proteins (CLPs), both of which bind chitin, a polysaccharide chain composed of N-acetylglucosamine repeats and present in arthropods and other taxa as a major structural polymer. While chitinases cleave chitin, CLPs do not possess this enzymatic activity. CHI3L1, one of the CLPs, also has been named YKL-40 in humans and breast regression protein 39 (BRP-39) in mice. Following its initial discovery in the culture supernatant of the osteosarcoma cell line MG63, it was subsequently detected in human chondrocytes, synoviocytes, and vascular smooth muscle cells. In fact, CHI3L1 is produced by a multitude of cells, including macrophages, neutrophils, fibroblast-like cells, hepatic stellate cells, endothelial cells, and cancer cells. It binds to chitin, heparin, and hyaluronic acid, and is regulated by extracellular matrix changes, cytokines, growth factors, drugs, and stress. CHI3L1 plays a major role in tissue injury, inflammation, tissue repair, and remodeling responses. See Zhao et al., 2020, Signal Transduction and Targeted Therapy Volume 5, Article number: 201, doi.org/10.1038/s41392-020-00303-7.

In some embodiments, the CHI3L1 protein is encoded by a polynucleotide comprising or consisting of SEQ ID NO: 1. In some embodiments, the CHI3L1 protein is encoded by a polynucleotide having at least 85%, 87%, 90%, 95%, 96%, 97%, 98% or 99% sequence identity to SEQ ID NO: 1. In some embodiments, the CHI3L1 protein comprises or consists of SEQ ID NO: 2. In some embodiments, the CHI3L1 protein comprises or consists of a polypeptide having at least 85%, 87%, 90%, 95%, 96%, 97%, 98% or 99% sequence identity to SEQ ID NO: 2.

In some embodiments, at least one of the therapeutic polypeptides is a fibroblast growth factor (FGF). FGFs and their receptors control a wide range of biological functions, regulating cellular proliferation, survival, migration and differentiation. FGFs have shown potential effects on the repair and regeneration of tissues. FGFs were originally identified as proteins capable of promoting fibroblast proliferation, and the protein family is now known to comprise 22 members, i.e., FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21 and FGF-22. FGFs exert multiple functions through the binding into and activation of fibroblast growth factor receptors (FGFRs), and the main signaling through the stimulation of FGFRs is the RAS/MAP kinase pathway. FGFs have been utilized for the regeneration of damaged tissues, including skin, blood vessel, muscle, adipose, tendon/ligament, cartilage, bone, tooth, and nerve. See Yun et al., 2010, J Tissue Eng., doi: 10.4061/2010/218142.

In some embodiments, the FGF is selected from the group consisting of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21 and FGF-22. In some embodiments, the FGF is selected from the group consisting of FGF-1, FGF-2, FGF-7, FGF-10 and FGF-21.

In some embodiments, at least one of the therapeutic polypeptides is FGF-2. In some embodiments, the FGF-2 protein is encoded by a polynucleotide comprising or consisting of SEQ ID NO: 3. In some embodiments, the FGF-2 protein is encoded by a polynucleotide having at least 85%, 87%, 90%, 95%, 96%, 97%, 98% or 99% sequence identity to SEQ ID NO: 3. In some embodiments, the FGF-2 protein comprises or consists of SEQ ID NO: 4. In some embodiments, the FGF-2 protein comprises or consists of a polypeptide having at least 85%, 87%, 90%, 95%, 96%, 97%, 98% or 99% sequence identity to SEQ ID NO: 4.

In some embodiments, at least one of the therapeutic polypeptides is IL-2 ORF7. In some embodiments, the IL-2 ORF7 polypeptide is encoded by a polynucleotide comprising or consisting of SEQ ID NO: 5. In some embodiments, the IL-2 ORF7 polypeptide is encoded by a polynucleotide having at least 85%, 87%, 90%, 95%, 96%, 97%, 98% or 99% sequence identity to SEQ ID NO: 5. In some embodiments, the IL-2 ORF7 polypeptide comprises or consists of SEQ ID NO: 6. In some embodiments, the IL-2 ORF7 polypeptide comprises or consists of a polypeptide having at least 85%, 87%, 90%, 95%, 96%, 97%, 98% or 99% sequence identity to SEQ ID NO: 6.

In some embodiments, at least one of the therapeutic polypeptides is IL-2. In some embodiments, the IL-2 polypeptide is encoded by a polynucleotide comprising or consisting of SEQ ID NO: 11. In some embodiments, the IL-2 polypeptide is encoded by a polynucleotide having at least 85%, 87%, 90%, 95%, 96%, 97%, 98% or 99% sequence identity to SEQ ID NO: 11. In some embodiments, the IL-2 polypeptide comprises or consists of SEQ ID NO: 12. In some embodiments, the IL-2 polypeptide comprises or consists of a polypeptide having at least 85%, 87%, 90%, 95%, 96%, 97%, 98% or 99% sequence identity to SEQ ID NO: 12.

In some embodiments, the IL-2 polypeptide does not comprise a signal sequence. The signal sequence of human IL-2 is the amino acid residues at positions 1-20 of SEQ ID NO: 12, and is encoded by the nucleic acid sequence at positions 1-60 of SEQ ID NO: 11. Accordingly, in some embodiments, the IL-2 polypeptide is encoded by a polynucleotide having at least 85%, 87%, 90%, 95%, 96%, 97%, 98% or 99% sequence identity to the nucleic acid sequence at positions 61-462 of SEQ ID NO: 11. In some embodiments, the IL-2 polypeptide comprises or consists of the amino acid sequence at positions 21-153 of SEQ ID NO: 12. In some embodiments, the IL-2 polypeptide comprises or consists of a polypeptide having at least 85%, 87%, 90%, 95%, 96%, 97%, 98% or 99% sequence identity to the amino acid sequence at positions 21-153 of SEQ ID NO: 12.

In some embodiments, at least one of the therapeutic polypeptides is an IL-17 protein or an IL-17 receptor. The IL-17 family is evolutionary conserved within vertebrates and composes six structurally related members in mammals, IL-17A, IL-17B, IL-17C, IL-17D, IL-17E, and IL-17F. Five IL-17 receptor subunits have been identified (IL-17RA, IL-17RB, IL-17RC, IL-17RD, and IL-17RE). All are single-pass transmembrane receptors with two extracellular fibronectin II-like domains and a cytoplasmic “SEFIR” motif for activation of downstream signaling pathways. All cytokines bind to their receptors as disulfide-linked homodimers, with the exception of IL-17A and IL-17F, which can also form heterodimers. IL-17R signal transduction has been studied for IL-17RA/IL-17RC complexes after IL-17A ligation. Receptor engagement leads to a conformational change, which allows the adaptor protein Act1 to form a homotypic interaction between the receptor's “SEFIR” domain and its own. The E3 ligase activity of Act1 initiates the canonical pathway via ubiquitination of TNF receptor -associated factor 6 (TRAF6), recruitment of TAK1 and subsequent activation of the canonical NF-κB and MAPK pathways including ERK, p38, and JNK, as well as the CCAAT-enhancer-binding proteins pathway. IL-17 is a pleiotropic cytokine that acts mainly on cells of mesenchymal origin such as endothelial cells, epithelial cells, and fibroblasts, but also has effects on immune cells, including dendritic cells and macrophages. IL-17 executes key functions to prevent pathogen invasion by the secretion of proinflammatory cytokines, chemokines, matrix metalloproteinases, growth factors, and antimicrobial peptides. See Zwicky et al., 2019, J Exp. Med. 217 (1): e20191123.

In some embodiments, at least one of the therapeutic polypeptides is an IL-17 receptor. In some embodiments, the IL-17 receptor is selected from the group consisting of IL-17RA, IL-17RB, IL-17RC, IL-17RD, and IL-17RE. In some embodiments, the IL-17 receptor is IL-17RE.

In some embodiments, at least one of the therapeutic polypeptides is an IL-17 protein. In some embodiments, the IL-17 protein is selected from the group consisting of IL-17A, IL-17B, IL-17C, IL-17D, IL-17E, and IL-17F. In some embodiments, the IL-17 protein is selected from the group consisting of IL-17A, IL-17C and IL-17F.

In some embodiments, at least one of the therapeutic polypeptides is IL-17A. In some embodiments, the IL-17A protein is encoded by a polynucleotide comprising or consisting of SEQ ID NO: 7. In some embodiments, the IL-17A protein is encoded by a polynucleotide having at least 85%, 87%, 90%, 95%, 96%, 97%, 98% or 99% sequence identity to SEQ ID NO: 7. In some embodiments, the IL-17A protein comprises or consists of SEQ ID NO: 8.

In some embodiments, the IL-17A protein comprises or consists of a polypeptide having at least 85%, 87%, 90%, 95%, 96%, 97%, 98% or 99% sequence identity to SEQ ID NO: 8.

In certain aspects, the present disclosure relates to combinations of two or more therapeutic polypeptides, and nucleic molecules encoding these therapeutic polypeptides. In some embodiments, the disclosure relates to a combination of CHI3L1 and FGF-2. In some embodiments, the disclosure relates to a combination of CHI3L1 and IL-2 ORF7. In some embodiments, the disclosure relates to a combination of CHI3L1 and IL-2. In some embodiments, the disclosure relates to a combination of CHI3L1 and IL-17A. In some embodiments, the disclosure relates to a combination of FGF-2 and IL-2 ORF7. In some embodiments, the disclosure relates to a combination of FGF-2 and IL-2. In some embodiments, the disclosure relates to a combination of FGF-2 and IL-17A. In some embodiments, the disclosure relates to a combination of IL-2 ORF7 and IL-17A. In some embodiments, the disclosure relates to a combination of IL-2 and IL-17A.

The two or more therapeutic polypeptides may be encoded by a single nucleic acid molecule, or by two or more nucleic acid molecules. For example, in some aspects, the disclosure relates to a recombinant nucleic acid molecule encoding two or more different therapeutic polypeptides. In some aspects, the disclosure relates to a combination of two or more recombinant nucleic acid molecules each encoding a different therapeutic polypeptide. For example, in some aspects, the disclosure relates to a pharmaceutical composition comprising: (a) two or more recombinant nucleic acid molecules (e.g., RNA molecules) each encoding a different polypeptide, wherein each of the polypeptides is selected from the group consisting of chitinase-3-like protein 1 (CHI3L1), fibroblast growth factor (FGF-2), interleukin-2 ORF7 (IL-2 ORF7), interleukin-2 (IL-2) and interleukin-17A (IL-17A); and (b) a pharmaceutically acceptable carrier.

In some embodiments, each recombinant nucleic acid molecule encodes only one therapeutic protein. In some embodiments, the recombinant nucleic acid molecule encodes two therapeutic proteins. In some embodiments, the recombinant nucleic acid molecule encodes three therapeutic proteins. In some embodiments, the recombinant nucleic acid molecule encodes four therapeutic proteins. In some embodiments, the recombinant nucleic acid molecule encodes at least two, or at least three, therapeutic proteins.

In some embodiments, the recombinant nucleic acid molecules encoding the therapeutic proteins are comprised within a pharmaceutical composition, vector, (e.g. an engineered virus, plasmid or transposon) or cell. In some embodiments, the pharmaceutical composition, vector, (e.g. an engineered virus, plasmid or transposon) or cell comprises 2, 3 or 4 nucleic acid molecules, each encoding a different therapeutic protein.

In some embodiments, the recombinant nucleic acid molecule comprises fewer than 100, 90, 80, 70, 60, 50, 40, 30, 20, 10 or 5 kb. In some embodiments, the recombinant nucleic acid molecule comprises at least 5, 10, 20, 30, 40, 50, 60, 70, 80, 90 or 100 kb. Any of these values may be used to define a range for the size of the recombinant nucleic acid molecule. For example, in some embodiments, the recombinant nucleic acid molecule comprises 5-100 kb or 5-50 kb.

Exemplary therapeutic protein sequences and polynucleotide sequences encoding the therapeutic proteins are provided in Table 1 below. Any other polynucleotide sequences that encode the therapeutic proteins of Table 1 (or encode polypeptides at least 85%, 87%, 90%, 95%, 97%, 98%, or 99% identical thereto) can also be used in the methods and compositions described herein. In some embodiments, the therapeutic protein is a wild type protein, or a functional fragment thereof. In some embodiments, the functional fragment is an N-terminal or C-terminal truncation of a wild type therapeutic protein. In some embodiments, the therapeutic proteins described herein may be mutated, for example, to further enhance their ability to promote wound healing. For example, in some embodiments, the therapeutic protein or functional fragment thereof comprises one or more mutations relative to the wild type protein.

TABLE 1
Exemplary therapeutic polypeptide sequences and polynucleotide
sequences encoding therapeutic proteins. ORF7 of the human IL-2 gene
is provided in NCBI Accession No. NM_000586.4.

| Therapeutic | | Nucleic Acid | Amino Acid |
|---|---|---|---|
| Protein | Uniprot | Sequence | Sequence |
| CHI3L1 | P36222 | SEQ ID NO: 1 | SEQ ID NO: 2 |
| FGF-2 | P09038 | SEQ ID NO: 3 | SEQ ID NO: 4 |
| IL-2 ORF7 | N/A | SEQ ID NO: 5 | SEQ ID NO: 6 |
| IL-2 | Q13169 | SEQ ID NO: 11 | SEQ ID NO: 12 |
| IL-17A | Q16552 | SEQ ID NO: 7 | SEQ ID NO: 8 |

### 2A Peptides

The recombinant nucleic acid molecule encoding the therapeutic protein may further comprise a polynucleotide encoding a 2A peptide. 2A peptides induce ribosomal skipping during translation of a protein, such that two proteins encoded by the same mRNA transcript may be expressed as separate proteins. See Liu et al., 2017, Scientific Reports. 7 (1): 2193, which is incorporated by reference herein in its entirety. These peptides share a core sequence motif, are about 18-22 amino acid residues in length, and are found in a wide range of viruses. Exemplary 2A peptides include, but are not limited to T2A, P2A, E2A and F2A. In a particular embodiment, the 2A peptide is a P2A peptide. The polynucleotide encoding the 2A peptide may be located between polynucleotides encoding two different therapeutic proteins (e.g., between polynucleotides encoding any two therapeutic proteins described herein) to allow for separate expression of each therapeutic protein.

### III. Modes of Administering Nucleic Acid Molecules

### A. DNA Delivery Methods

In some embodiments, the recombinant nucleic acid molecules as described herein are delivered to a subject as DNA. In some embodiments, the recombinant DNA molecules delivered to the subject are not comprised within a virus, cell, bacterium, or other organism.

For example, in some embodiments, the recombinant nucleic acid molecule is comprised within a DNA plasmid. In some embodiments, the recombinant DNA molecules encoding the therapeutic proteins are comprised within a transposon.

The polynucleotides encoding the therapeutic proteins may each be operably linked to a promoter. In some embodiments, the promoter is a polymerase II (Pol II) promoter. Suitable Pol II promoters include but are not limited to a cytomegalovirus (CMV) promoter or an SV40 promoter (e.g. pcDNA3.1, pVAX1, pVIVO2, pCI, pCMV and pSV2). In a particular embodiment, the promoter is a cytomegalovirus (CMV) promoter, an EF1a promoter, or a UBC1 promoter. In some embodiments, the promoter is a tissue-specific promoter. In some embodiments, the promoter is a synthetic promoter. Suitable promoters for DNA delivery are known in the art and are described, for example, in Li, L, et al., 2016, Expert Rev Vaccines 15:313-29, which is incorporated by reference herein in its entirety. In some embodiments, the promoter is selected from the group consisting of a CMV promoter (e.g., a mini-CMV promoter), an EF1a promoter (e.g., a mini-EF1a promoter), an SV40 promoter, a PGK1 promoter, a polyubiquitin C (UBC) gene promoter, a human beta actin promoter, and a CMV enhancer/chicken beta-actin/rabbit beta-globin (CAG) hybrid promoter. In some embodiments, the promoter is a cancer-specific promoter, e.g., a tumor-specific promoter. Suitable tumor-specific promoters include, but are not limited to, a human telomerase reverse transcriptase (hTERT) promoter and an E2F promoter. The hTERT promoter drives gene expression in cells (such as cancer cells) with increased expression of telomerase. The E2F promoter drives gene expression that is specific to cells with an altered Rb pathway.

The recombinant DNA molecules encoding the therapeutic proteins may each be operably linked to a DNA sequence encoding a 3′ polyadenylation (poly A) signal. In some embodiments, the poly A signal is a rabbit β-globin poly A signal or a bovine growth hormone poly A signal. The poly A signal is involved in nuclear export, translation and stability of the transcript mRNA. See Williams, J A, et al. 2013, Vaccines 1:225-49.

Methods of formulating the DNA for delivery to a subject include, but are not limited to, encapsulation in lipid nanoparticles containing cationic lipids and cholesterol, adsorption to polymers such as polyethyleneimine, and adsorption or encapsulation in biodegradable nanoparticles, such as poly(lactic-co-glycolic acid) (PLGA) or chitosan. See Donnelly J J, et al., 2005, J Immunol. 175:633-9.

The sequences of the recombinant nucleic acid molecules encoding the therapeutic proteins may be codon optimized, e.g., by using enrichment of the GC content (see Thess A, et al., 2015, Mol Ther. 23:1456-64; Petsch B et al., 2012, Nat Biotechnol. 30:1210-6; and Kudla G et al., 2006, PLoS Biol. 4:e180. doi: 10.1371/journal.pbio.0040180) and/or by replacement of rare codons. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments a codon-optimized DNA may, for instance, be one in which the levels of G/C are enhanced. The G/C-content of nucleic acid molecules may influence the stability of the corresponding RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the DNA/RNA.

The recombinant DNA molecules encoding the therapeutic proteins may be delivered to a subject with synthetic delivery vehicles, such as lipid nanoparticles (LNPs). Lipid nanoparticles suitable for DNA molecule delivery are known in the art and are described, for example, in Reichmuth A M, et al., 2016, Ther Deliv. 7(5):319-334; Geall A J, et al., 2012, Proc Natl Acad Sci USA. 109:14604-9; and U.S. Pat. No. 10,702,600, each of which is incorporated by reference herein in its entirety. Suitable lipids and lipid complexes for use in lipid nanoparticles include, but are not limited to, DLinDMA: 1,2-dilinoleyloxy-3-dimethylaminopropane; DOPE: 1,2-dioleoyl-sn-glycero-3-phosphoethanolamine; DOTAP: 1,2-Dioleyl-3-trimethylammonium-propane chloride salt; DSPC: 1,2-Diastearoyl-sn-glycero-3-phosphocholine; Histidylated lipoplex: PEGylated derivative of histidylated polylysine and L-histidine-(N,N-di-n-hexadecylamine)ethylamide liposomes; HVJ-liposome: liposome with fusion proteins derived from the hemagglutinating virus of Japan (HVJ); Man11-LPR100: Mannosylated and histidylated lipopolyplexes (Man11-LPR100) obtained by adding mannosylated and histidylated liposomes to mRNA-PEGylated histidylated polylysine polyplexes; PC: Dipalmitoylphosphatidylcholine; cholesterol, PEG DMG 2000: 1,2-dimyristoyl-sn-glycero-3-phosphoethanolamine-N-[methoxy(polyethylene glycol)-2000]; PS: Phosphatidylserine; Span 85: sorbitane trioleate; unifectin; and squalene. See Martinon F, et al., 1993, Eur. J. Immunol. 23(7), 1719-1722; Hess P R, et al., 2005, Cancer Immunol. Immunother. 55(6), 672-683. Zhou W-Z, et al., 1999. Hum. Gene Ther. 10(16), 2719-2724; Pollard C, et al., 2013, Mol. Ther. 21(1), 251-259; Hoerr I, et al., 2000, Eur. J. Immunol. 30(1), 1-7; Mockey M, et al., 2007, Cancer Gene Ther. 14(9), 802-814; Perche F, et al., 2011, RNA. Nanomed. Nanotechnol. Biol. Med. 7(4), 445-453; Phua K K L, et al., 2014, Sci. Rep. 4, 5128; Geall A J, et al., 2012, Proc. Natl Acad. Sci. USA 109(36), 14604-14609; and Brito L A, et al., 2014, Mol. Ther. 22(12), 2118-2129.

In some embodiments, the lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the lipid is (L608).

The DNA molecules may also be formulated using liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties).

In some embodiments, the DNA molecules may be formulated in a lipid vesicle, which may have crosslinks between functionalized lipid bilayers. In some embodiments, the DNA molecules may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the DNA molecules may be formulated in a lipid-polycation complex, which may further include a non-cationic lipid such as, but not limited to, cholesterol or dioleoyl phosphatidylethanolamine (DOPE).

In other embodiments, the recombinant DNA molecules encoding the therapeutic proteins may be packaged and delivered in virus-like replicon particles (VRPs) produced by a helper cell line that provides the capsid and glycoprotein genes in trans. In some embodiments, the DNA molecules are delivered to a subject as free DNA, i.e. they are not complexed to another molecule. In some embodiments, the DNA molecules are delivered to a subject as protamine-complexed DNA. Protamine is a natural cationic nuclear protein expressed in testis. It is a highly specialized molecule that replaces histones during the final condensation of DNA in sperm and is known to stabilize nucleic acids. It has an arginine-rich sequence and spontaneously associates with nucleic acids in vitro. Protamine-complexed DNA provides both strong gene expression and immunostimulation. See Scheel B et al., 2005, Eur J Immunol. 35:1557-66; Fotin-Mleczek M, 2011, J Immunother. 34:1-15; Fotin-Mleczek M, et al., 2012, J Gene Med. 14:428-39; and Kowalczyk A, et al., 2016, Vaccine 34:3882-93.

### B. RNA Delivery Methods

In certain aspects, the disclosure relates to a recombinant RNA molecule (e.g., mRNA) encoding one or more polypeptides selected from the group consisting of chitinase-3-like protein 1 (CHI3L1), a fibroblast growth factor, interleukin-2 ORF7 (IL-2 ORF7), an interleukin-17 (IL-17) protein, interleukin-2 (IL-2) and an IL-17 receptor. In some embodiments the recombinant RNA molecule comprises at least one chemical modification.

In some embodiments, the recombinant RNA molecule is an isolated recombinant RNA molecule.

In certain aspects, the disclosure relates to a recombinant RNA molecule (e.g., mRNA) encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A). In some embodiments the recombinant RNA molecule comprises at least one chemical modification.

In some embodiments, the RNA is not comprised within a virus or cell. In some embodiments, the RNA is not comprised within a bacterium, or other organism. In some embodiments, the RNA is purified, e.g., HPLC-purified. In some embodiments, the RNA is a circular RNA.

In some embodiments, the RNA is mRNA. The one or more mRNAs encoding the one or more therapeutic polypeptides may be operably linked to 5′ and/or 3′ untranslated regions (UTRs). The UTRs, which can be of eukaryotic or viral origin, increase the half-life, and stability of the mRNA, resulting in higher expression of the therapeutic protein (see Ross J, et al., 1985, Blood 66:1149-54; Gallie D R, et al., 1995, Gene 165:233-8; Kariko K, et al., 2012 Mol Ther. 20: 948-53; and Vivinus S, et al. 2001, Eur J Biochem. 268:1908-17).

A cap structure may be operably linked to the 5′ end of the mRNA. The cap structure is an N7-methylated guanosine linked to the first nucleotide of the mRNA via a reverse 5′ to 5′ triphosphate linkage. In addition to its role in cap-dependent initiation of protein synthesis, the mRNA cap also functions as a protective group from 5′ to 3′ exonuclease cleavage and a unique identifier for recruiting protein factors for pre-mRNA splicing, polyadenylation and nuclear export. See Ramanathan A, et al., 2016, Nucleic Acids Res. 44(16): 7511-7526. The 5′ cap structure is important for the creation of stable mature mRNA, and increases protein translation via binding to eukaryotic translation initiation factor 4E. See Gallie, D R., 1991, Genes Dev. 5:2108-16. The 5′ cap may be added either during transcription by inclusion of a cap analog or antireverse cap (ARCA) in the reaction (see Stepinski J, et al., 2001, RNA 7:1486-95) or subsequently, using the Vaccinia virus capping complex (see Venkatesan S, et al. 1980, J Biol Chem. 255, 903-908). In some embodiments, the 5′ terminal cap is 7mG(5′)ppp(5′)NlmpNp.

A poly(A) tail may be operably linked to the 3′ end of the mRNA. The poly A tail is an important regulatory element to enhance translation and can be either be encoded by the DNA template or alternatively added enzymatically post transcription (Gallie, D R., 1991, Genes Dev. 5:2108-16).

The sequence of an mRNA encoding a therapeutic polypeptide (e.g., CHI3L1, FGF-2, IL-2 ORF7, IL-2 or IL-17A) may be codon optimized, e.g. by using either enrichment of the GC content (see Thess A, et al., 2015, Mol Ther. 23:1456-64; Petsch B et al., 2012, Nat Biotechnol. 30:1210-6; and Kudla G et al., 2006, PLoS Biol. 4:e180. doi: 10.1371/journal.pbio.0040180) or by replacement of rare codons. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Chemically modified nucleosides may be added to the RNA (e.g. mRNA), for example. to decrease innate immune activation and/or increase translation of the RNA (e.g. mRNA). See Kariko K, et al., 2008, Mol Ther. 16:1833-40; and U.S. Pat. No. 10,702,600. In some embodiments, the RNA (e.g. mRNA) has an open reading frame encoding at least one polypeptide that comprises at least one chemical modification.

The terms “chemical modification” and “chemically modified” refer to modification relative to a wildtype nucleic acid molecule with respect to adenosine (A), guanosine (G), uridine (U), thymidine (T) or cytidine (C) ribonucleosides or deoxyribnucleosides in at least one of their position, pattern, percent or population. In some embodiments, a chemical modification is a replacement of a nucleoside with a different form of the same nucleoside, e.g., a replacement of uridine with pseudouridine, or a replacement of cytidine with 5-methyl-cytidine. In some embodiments, a chemical modification is a replacement of a nucleoside with a different nucleoside, e.g., a replacement of uridine with cytidine. Generally, these terms do not refer to the ribonucleotide modifications in naturally occurring 5′-terminal mRNA cap moieties. With respect to a polypeptide, the term “modification” refers to a modification relative to the canonical set of 20 amino acids. Polypeptides, as provided herein, are also considered “modified” if they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside plus a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides) that are useful in the RNA molecules of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)pseudouracil; 2′ methyl, 2′amino, 2′ azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±) 1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene;2 (amino)purine;2,4,5-(trimethyl)phenyl;2′ methyl, 2′amino, 2′azido, 2′fluoro-cytidine;2′ methyl, 2′ amino, 2′azido, 2′fluoro-adenine;2′methyl, 2′amino, 2′ azido, 2′fluoro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, RNA molecules (e.g., mRNA molecules) include a combination of at least two (e.g., 2, 3, 4 or more) of the aforementioned modified nucleobases.

In some embodiments, modified nucleobases in RNA molecules (e.g., mRNA molecules) are selected from the group consisting of pseudouridine (p), N1-methylpseudouridine (m¹ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in RNA molecules (e.g., mRNA molecules) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides include a combination of at least two (e.g., 2, 3, 4 or more) of the aforementioned modified nucleobases.

In some embodiments, RNA molecules (e.g., mRNA molecules) comprise pseudouridine (v) and 5-methyl-cytidine (m⁵C). In some embodiments, RNA molecules (e.g., mRNA molecules) comprise 1-methyl-pseudouridine (m¹ψ). In some embodiments, RNA molecules (e.g., mRNA molecules) comprise 1-methyl-pseudouridine (m¹ψ) and 5-methyl-cytidine (m⁵C). In some embodiments, RNA molecules (e.g., mRNA molecules) comprise 2-thiouridine (s²U). In some embodiments, RNA molecules (e.g., mRNA molecules) comprise 2-thiouridine and 5-methyl-cytidine (m⁵C). In some embodiments, RNA molecules (e.g., mRNA molecules) comprise methoxy-uridine (mo⁵U). In some embodiments, RNA molecules (e.g., mRNA molecules) comprise 5-methoxy-uridine (mo⁵U) and 5-methyl-cytidine (m⁵C). In some embodiments, RNA molecules (e.g., mRNA molecules) comprise 2′-O-methyl uridine. In some embodiments RNA molecules (e.g., mRNA molecules) comprise 2′-O-methyl uridine and 5-methyl-cytidine (m⁵C). In some embodiments, RNA molecules (e.g., mRNA molecules) comprise N6-methyl-adenosine (m⁶A). In some embodiments, RNA molecules (e.g., mRNA molecules) comprise N6-methyl-adenosine (m⁶A) and 5-methyl-cytidine (m⁵C).

In some embodiments, RNA molecules (e.g., mRNA molecules) are uniformly modified (e.g., fully modified, modified throughout the entire sequence) for a particular modification. For example, an RNA molecule can be fully modified with 5-methyl-cytidine (m⁵C), meaning that all cytidine residues in the mRNA sequence are replaced with 5-methyl-cytidine (m⁵C). In some embodiments, the RNA molecule is fully modified with pseudouridine. Similarly, an RNA molecule can be fully modified for any type of nucleoside residue present in the sequence by replacement with a modified residue such as those set forth above. In some embodiments, two, three or four types of nucleoside residues present in the RNA molecule may be fully modified with a chemical modification described herein. For example, in some embodiments the RNA molecule (e.g., mRNA molecule) is fully modified with pseudouridine and 5-methyl-cytidine (m⁵C), i.e., all uridine residues are replaced with pseudouridine and all cytidine residues are replaced with 5-methyl-cytidine (m⁵C).

Exemplary nucleobases and nucleosides having a modified cytidine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleoside is a modified uridine. Nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (mlG), 8-oxo-guanosine, 7-methyl-8-oxo-guanosine.

The nucleic acid molecules of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid molecule of the disclosure, or in a given predetermined sequence region thereof (e.g., in the mRNA including or excluding the polyA tail). In some embodiments, all nucleotides X in a nucleic acid molecule of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The nucleic acid molecule may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

Thus, in some embodiments, the RNA (e.g., mRNA) molecules comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(m⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m5s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (msUm), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴2Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g⁶A), N6,N6-dimethyl-adenosine (m⁶2A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (mG), N2-methyl-guanosine (m²G), N2,N2-dimethyl-guanosine (m²2G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²2Gm), 1-methyl-2′-O-methyl-guanosine (mGm), N2,7-dimethyl-2′-O-methyl-guanosine (m²′7Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

In some embodiments, the mRNA is non-replicating mRNA. In other embodiments, the mRNA is self-amplifying mRNA. Self-amplifying mRNA may be based on an alphavirus genome, which contains the genes encoding the alphavirus RNA replication machinery, but lacks the genes encoding the viral structural proteins required to make an infectious alphavirus particle. See Geall A J et al., 2012, Proc Natl Acad Sci USA 109(36): 14604-14609. The structural protein genes of the alphavirus may be replaced with one or more nucleic acid molecules encoding one or more therapeutic polypeptides, (e.g., one or more of CHI3L1, FGF-2, IL-2 ORF7 and IL-17A), which are abundantly expressed from a subgenomic mRNA in the cytoplasm of cells transfected with these self-amplifying RNAs. The self-amplifying mRNAs may be produced in vitro by an enzymatic transcription reaction from a linear pDNA template using a T7 RNA polymerase, thereby avoiding safety concerns and complex manufacturing issues associated with cell culture production of live viral vaccines, recombinant subunit proteins, and viral vectors. After immunization, replication and amplification of the mRNA molecule occurs exclusively in the cytoplasm of the transfected cells, thereby eliminating risks of genomic integration and cell transformation. See Brito L A, et al. 2015, Adv Genet. 89:179-233; Perri S, et al. 2003, J Virol. 77:10394-403; and Geall A J, et al., 2012, Proc Natl Acad Sci USA. 109:14604-9.

The full length mRNA of the self-amplifying mRNA is substantially larger (approximately 9-10 kb for alphavirus systems) than in non-replicating mRNAs but contains the same essential elements such as a cap, 5′ and 3′ UTRs, and poly A tail as described above. The DNA encoding the self-amplifying mRNA comprises a sub-genomic promoter and a large ORF encoding nonstructural viral proteins which, following delivery of the DNA into the cytosol, are transcribed in four functional components (nsP1, nsP2, nsP3, and nsp4) by the encoded RNA-dependent RNA polymerase (RDRP) (see Iavarone C, et al., 2017, Expert Rev Vaccines 16:871-81). RDRP then produces a negative-sense copy of the genome which serves as a template for two positive strand RNA molecules: the genomic mRNA and a shorter sub-genomic mRNA. This sub-genomic mRNA is transcribed at very high levels, allowing the amplification of mRNA encoding the polypeptide of choice.

In some embodiments, the mRNAs may be codon optimized to modulate their stability. For example, in some embodiments, a codon optimized mRNA has increased stability relative to a corresponding mRNA that is not codon optimized. In some embodiments, a codon optimized mRNA has decreased stability relative to a corresponding mRNA that is not codon optimized. For example, the mRNA encoding the therapeutic polypeptide (e.g., CHI3L1, FGF-2, IL-2 ORF7, IL-2 or IL-17A) may be codon optimized to increase it stability. Methods of codon optimizing mRNA to modulate stability are known in the art and are described, for example, in Bicknell A A et al., 2017, Biochem Soc Trans. 45(2):339-351; Radhakrishnan A, et al, 2016, J Mol Biol. 428(18):3558-3564; Chen Y H, et al., 2016, Trends Genet. 2016; 32(11):687-688; and Hanson G, et al., 2018, Nat Rev Mol Cell Biol. 19(1):20-30.

The mRNA may comprise one or more modified nucleotides, e.g. to modulate its stability. In some embodiments, the one or more modified nucleotides increase stability of the mRNA relative to a corresponding mRNA that does not comprise the one or more modified nucleotides. In some embodiments, the one or more modified nucleotides decrease stability of the mRNA relative to a corresponding mRNA that does not comprise the one or more modified nucleotides. Suitable modified nucleotides include, but are not limited to, N6-methyladenosine (m6A), N6,2′-O-dimethyladenosine (m6Am), 5-methylcytidine (m5C), inosine (I), pseudouridine (Ψ), N1-methyladenosine (m1A), 5-hydroxylmethylcytidine (hm5C), 2′-O-methylation (Nm), and N4-Acetylcytidine. See Roundtree I A, et al., 2017, Cell 169(7):1187-1200; and Li X, et al., 2019, Biochemistry 58(12):1553-1554.

The mRNA may comprises a protein binding site in a 3′-UTR of the mRNA. The protein binding site may decreases stability of the mRNA. In some embodiments, the protein binding site is a Staufen1 (STAU1)-mediated binding site (SBS). See Park E, et al., 2013, Wiley Interdiscip Rev RNA. 4(4):423-435; and Chen Y H, et al., 2016, Trends Genet. 32(11):687-688. Staufen1 (STAU1)-mediated mRNA decay (SMD) is an mRNA degradation process in mammalian cells that is mediated by the binding of STAU1 to a STAU1-binding site (SBS) within the 3′-untranslated region (3′-UTR) of target mRNAs. During SMD, STAU1, a double-stranded (ds) RNA-binding protein, recognizes dsRNA structures formed either by intramolecular base pairing of 3′-UTR sequences or by intermolecular base pairing of 3′-UTR sequences with a long-noncoding RNA (lncRNA) via partially complementary Alu elements. STAU1 interacts directly with the ATP-dependent RNA helicase UPF1, a key SMD factor, enhancing its helicase activity to promote effective STAU1-mediated mRNA decay. In some embodiments, the composition comprising one or more mRNAs encoding one or more therapeutic polypeptides (e.g., one or more of CHI3L1, FGF-2, IL-2 ORF7, IL-2 and IL-17A) further comprises a microRNA (miRNA) or a polynucleotide encoding an miRNA. The miRNA may decrease stability of the mRNA. In some embodiments, the miRNA is complementary to the mRNA encoding the therapeutic polypeptide (e.g., CHI3L1, FGF-2, IL-2 ORF7, IL-2 or IL-17A). The miRNA may be co-expressed with the mRNA encoding the therapeutic polypeptide (e.g., CHI3L1, FGF-2, IL-2 ORF7, IL-2 or IL-17A).

mRNA molecules encoding one or more therapeutic polypeptides (e.g., one or more of CHI3L1, FGF-2, IL-2 ORF7, IL-2 and IL-17A) may be delivered to a subject with synthetic delivery vehicles, such as lipid nanoparticles. Lipid nanoparticles for mRNA molecule delivery are known in the art and are described, for example, in Reichmuth A M, et al., 2016, Ther Deliv. 7(5):319-334; Geall A J, et al., 2012, Proc Natl Acad Sci USA. 109:14604-9; and U.S. Pat. No. 10,702,600, each of which is incorporated by reference herein in its entirety. Suitable lipids and lipid complexes for use in lipid nanoparticles include, but are not limited to, DLinDMA: 1,2-dilinoleyloxy-3-dimethylaminopropane; DOPE: 1,2-dioleoyl-sn-glycero-3-phosphoethanolamine; DOTAP: 1,2-Dioleyl-3-trimethylammonium-propane chloride salt; DSPC: 1,2-Diastearoyl-sn-glycero-3-phosphocholine; Histidylated lipoplex: PEGylated derivative of histidylated polylysine and L-histidine-(N,N-di-n-hexadecylamine)ethylamide liposomes; HVJ-liposome: liposome with fusion proteins derived from the hemagglutinating virus of Japan (HVJ); Man11-LPR100: Mannosylated and histidylated lipopolyplexes (Man11-LPR100) obtained by adding mannosylated and histidylated liposomes to mRNA-PEGylated histidylated polylysine polyplexes; PC: Dipalmitoylphosphatidylcholine; cholesterol, PEG DMG 2000: 1,2-dimyristoyl-sn-glycero-3-phosphoethanolamine-N-[methoxy(polyethylene glycol)-2000]; PS: Phosphatidylserine; Span 85: sorbitane trioleate; unifectin; and squalene. See Martinon F, et al., 1993, Eur. J. Immunol. 23(7), 1719-1722; Hess P R, et al., 2005, Cancer Immunol. Immunother. 55(6), 672-683. Zhou W-Z, et al., 1999. Hum. Gene Ther. 10(16), 2719-2724; Pollard C, et al., 2013, Mol. Ther. 21(1), 251-259; Hoerr I, et al., 2000, Eur. J. Immunol. 30(1), 1-7; Mockey M, et al., 2007, Cancer Gene Ther. 14(9), 802-814; Perche F, et al., 2011, RNA. Nanomed. Nanotechnol. Biol. Med. 7(4), 445-453; Phua K K L, et al., 2014, Sci. Rep. 4, 5128; Geall A J, et al., 2012, Proc. Natl Acad. Sci. USA 109(36), 14604-14609; and Brito L A, et al., 2014, Mol. Ther. 22(12), 2118-2129.

In some embodiments, the lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the lipid is (L608).

The RNA molecules may also be formulated using liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties).

In some embodiments, the RNA (e.g., mRNA) molecules may be formulated in a lipid vesicle, which may have crosslinks between functionalized lipid bilayers. In some embodiments, the RNA (e.g., mRNA) molecules may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the RNA (e.g., mRNA) molecules may be formulated in a lipid-polycation complex, which may further include a non-cationic lipid such as, but not limited to, cholesterol or dioleoyl phosphatidylethanolamine (DOPE).

In other embodiments, the mRNA molecules may be packaged and delivered in virus-like replicon particles (VRPs) produced by a helper cell line that provides the capsid and glycoprotein genes in trans. In some embodiments, the mRNA molecule is delivered to a subject as free mRNA, i.e. it is not complexed to another molecule. In some embodiments, the mRNA molecule is delivered to a subject as protamine-complexed mRNA. Protamine is a natural cationic nuclear protein expressed in testis. It is a highly specialized molecule that replaces histones during the final condensation of DNA in sperm and is known to stabilize nucleic acids. It has an arginine-rich sequence and spontaneously associates with nucleic acids in vitro. Protamine-complexed mRNA provides both strong gene expression and immunostimulation. See Scheel B et al., 2005, Eur J Immunol. 35:1557-66; Fotin-Mleczek M, 2011, J Immunother. 34:1-15; Fotin-Mleczek M, et al., 2012, J Gene Med. 14:428-39; and Kowalczyk A, et al., 2016, Vaccine 34:3882-93.

### C. Cell-Based Delivery Methods

In certain aspects, the disclosure relates to a cell comprising a recombinant RNA molecule encoding a therapeutic protein (e.g., CHI3L1, FGF-2, IL-2 ORF7, IL-2 and/or IL-17A) as described herein. In some embodiments, the cell is selected from the group consisting of a fibroblast an endothelial cell, and an immune cell.

In some embodiments, the cell is a fibroblast. A fibroblast is a type of cell that contributes to the formation of connective tissue, a fibrous cellular material that supports and connects other tissues or organs in the body. Fibroblasts secrete collagen proteins that help maintain the structural framework of tissues. They also play an important role in healing wounds. For example, fibroblasts are critical in supporting normal wound healing, and are involved in key processes such as breaking down the fibrin clot, and creating new extra cellular matrix (ECM) and collagen structures to support the other cells associated with effective wound healing. Fibroblasts are also involved in contracting the wound.

In some embodiments, the cell is an endothelial cell. Endothelial cells constitute the vascular endothelium, the inner cellular lining of arteries, veins and capillaries. Endothelial cells direct inflammatory cells to foreign materials and to areas in need of repair, such as wound. They also maintain the balance between coagulation and fibrinolysis, and play a major role in the regulation of immune responses, inflammation and angiogenesis. For example, endothelial cells play a crucial role in the regulation of inflammation during the early stages of wound healing. The loosening of cell-cell contacts and the expression of adhesion molecules allows endothelial cells to facilitate the movement of circulating inflammatory cells into the tissue at the site of injury.

In some embodiments, the cell is an immune cell. Immune cells include, but are not limited to, mast cells, Natural Killer (NK) cells, basophils, neutrophils, monocytes, macrophages, dendritic cells, eosinophils and lymphocytes. Types of lymphocytes include B-lymphocytes (B-cells) and T-lymphocytes (T-cells). In some embodiments, the immune cell is a macrophage or a T cell.

In some embodiments, the immune cell is a macrophage. Macrophages engulf and digest substances such as cellular debris, foreign substances, microbes and cancer cells in a process called phagocytosis. Besides phagocytosis, macrophages play a critical role in nonspecific defense (innate immunity) and also help initiate specific defense mechanisms (adaptive immunity) by recruiting other immune cells such as lymphocytes. For example, macrophages are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

In some embodiments, the immune cell is a T cell. T cells, derived from precursors in hematopoietic tissue, undergo differentiation in the thymus, and are then seeded to peripheral lymphoid tissue and to the recirculating pool of lymphocytes. T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)). T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4+ T cells); and those that express CD8 (CD8+ T cells). These cells differ in how they recognize antigen and in their effector and regulatory functions. CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. In addition, T cells, particularly CD8+ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)). T cells can also be classified based on their function as helper T cells; T cells involved in inducing cellular immunity; suppressor T cells (e.g., regulatory T cells); and cytotoxic T cells.

### D. Viral Delivery Methods

In certain aspects the disclosure relates to a virus engineered to comprise one or more recombinant nucleic acid molecules encoding one or more therapeutic proteins as described herein. Any virus that has the capacity to transfer a nucleic acid molecule encoding one or more therapeutic proteins into a target cell may be used. For example, in some embodiments, the virus is capable of transporting a heterologous polynucleotide of at least 4, 5, 6, 7, 8, 9 or 10 kb into a target cell. In some embodiments, the virus is capable of transporting a heterologous polynucleotide of between 4-12 kb into a target cell. In some embodiments, the virus is cytolytic, i.e., capable of lysing the target cell.

The virus may be a DNA virus or an RNA virus (e.g., a retrovirus). In some embodiments, the virus is an RNA virus. In some embodiments, the virus is a DNA virus. In some embodiments, the virus is a replicative virus. In some embodiments, the virus is a non-replicative virus. In some embodiments, the DNA virus is a DNA replicative virus, e.g. a DNA replicative oncolytic virus. In some embodiments, the RNA virus is a RNA replicative virus, e.g., a RNA replicative oncolytic virus. In some embodiments, the virus is an anellovirus.

In some embodiments, the virus is a wildtype virus. In some embodiments, the virus is a recombinant virus. In some embodiments, the virus is a pseudotyped virus. The term “pseudotyped virus” as used herein refers to a viral particle formed with a structural and enzymatic core from one virus and the envelope glycoprotein of another. In some embodiments, the pseudotyped virus is replication defective. In some embodiments, the pseudotyped virus is replication competent.

In some embodiments, the virus is capable of reinfecting a host that was previously infected with the virus. This characteristic allows for multiple administrations of the virus to a subject.

In some embodiments, it is advantageous for the virus to comprise an inactivating mutation in one or more endogenous viral genes. In some embodiments, the inactivating mutation is in an endogenous viral gene that contributes to virulence of the virus, such that the inactivating mutation decreases virulence. In some embodiments, the inactivating mutation is in an endogenous viral gene that restricts turnover of the infected cell, such that the inactivating mutation facilitates or increases turnover of the cell upon infection. In some embodiments, inactivating mutations in viral genes may be combined with expression of additional polynucleotides or polypeptides that modulate virulence or cell turnover.

In some embodiments, the virus engineered to comprise one or more polynucleotides encoding a therapeutic protein is selected from the group consisting of adenovirus, herpes simplex virus (HSV), poxyvirus (e.g., Vaccinia virus), a respiratory syncytial virus (RSV), adeno-associated virus (AAV), Coxsackievirus, Newcastle disease virus, Measles Virus, Myxomatosis, Poliovirus, Lentivirus, Vesicular Stomatitis Virus, a retrovirus, foamy virus, farmington virus, Parvoviruses, and influenza virus. In some embodiments, the virus engineered to comprise one or more polynucleotides encoding a therapeutic protein is selected from the group consisting of adenovirus and AAV.

In some embodiments, the virus engineered to comprise one or more polynucleotides encoding a therapeutic protein is an adenovirus. In some embodiments, the adenovirus is adenovirus serotype 5 (Ad5). In some embodiments, the adenovirus is adenovirus serotype 19A (Ad19A). In some embodiments, the adenovirus is adenovirus serotype 26 (Ad26). An adenovirus of one serotype may be engineered to comprise a fiber protein from a different adenovirus serotype. For example, in some embodiments, Ad5 is engineered to substitute the fiber protein from adenovirus serotype 35 (Ad35). This chimeric virus is referred to as Ad5/F35. (See Yotnda et al., 2001, Gene Therapy 8: 930-937, which is incorporated by reference herein in its entirety.) In some embodiments, Ad5 is engineered to substitute the fiber protein from adenovirus serotype 3 (Ad3). This chimeric virus is referred to as Ad5/F3.

In some embodiments, the adenovirus comprises one or more mutations (e.g., one or more substitutions, additions or deletions) relative to a corresponding wildtype adenovirus. For example, in some embodiments, the adenovirus (e.g., Ad5 or Ad5/F35) comprises a deletion in the Adenovirus Early Region 1A (E1A). In some embodiments, the adenovirus (e.g., Ad5 or Ad5/F35) comprises a 24 bp deletion in E1A. This deletion makes viral replication specific to cells with an altered Rb pathway. In some embodiments, the adenovirus (e.g., Ad5 or Ad5/F35) comprises a deletion in the Adenovirus Early Region 1B (E1B). In some embodiments, the adenovirus (e.g., Ad5 or Ad5/F35) comprises a 827 bp deletion in E1B. This deletion allows the virus to replicate in cells with P53 alterations. In a particular embodiment, the adenovirus (e.g., Ad5 or Ad5/F35) comprises a 24 bp deletion in E1A and a 827 bp deletion in E1B. In some embodiments, the adenovirus (e.g., Ad5 or Ad5/F35) has an Arg-Gly-Asp (RGD)-motif engineered into the fiber-H loop. This modification makes the adenovirus use αvβ3 and αvβ5 integrins (which are expressed in cancer cells) to enter the cell. (See Reynolds et al., 1999, Gene Therapy 6: 1336-1339, which is incorporated by reference herein in its entirety.)

In some embodiments, the adenovirus contains a modified or mutated fiber region. The modified or mutated fiber region may enhance or alter virus tropism and receptor binding.

In some embodiments a polynucleotide as described herein (e.g., a polynucleotide encoding a therapeutic protein) may be inserted into the E1 region of the adenovirus, e.g. in E1A or E1B. For example, in some embodiments the E1 region is removed and replaced with the polynucleotide. The polynucleotide may be operably linked to a promoter as described herein, e.g., a promoter that is heterologous to the virus. In some embodiments, a polynucleotide as described herein (e.g., a polynucleotide encoding a therapeutic protein) may be inserted downstream of an endogenous viral promoter to drive expression of the polynucleotide. For example, in some embodiments, the polynucleotide is inserted into an adenovirus downstream of the adenovirus major late promoter, which drives L5 protein expression. The adenovirus major late promoter confers expression concomitant with late viral gene expression. In some embodiments, the polynucleotide is inserted downstream of the endogenous viral gene encoding the L5 protein. In some embodiments, expression of the polynucleotide is linked to L5 expression using a 2A linker disposed between the polynucleotide and the gene encoding the L5 protein. In some embodiments, expression of the polynucleotide is linked to L5 expression by preceding the polynucleotide with an adenoviral splice acceptor under the control of the adenovirus major late promoter.

In some embodiments, the virus engineered to comprise one or more polynucleotides encoding a therapeutic protein is a herpes simplex virus (HSV), e.g. HSV1. In some embodiments, the HSV1 is selected from Kos, F1, MacIntyre, McKrae and related strains. The HSV may be defective in one or more genes selected from ICP6, ICP34.5, ICP47, UL24, UL55, and UL56. In a particular embodiment, the ICP34.5 encoding gene is replaced by a polynucleotide cassette comprising a US11 encoding gene operably linked to an immediate early (IE) promoter.

In other embodiments, the virus belongs to the Poxviridae family, e.g. a virus selected from myxoma virus, Yaba-like disease virus, raccoonpox virus, orf virus and cowpox virus. In some embodiments, the virus belongs to the Chordopoxvirinae subfamily of the Poxviridae family. In some embodiments, the virus belongs to the Orthopoxvirus genus of the Chordopoxvirinae subfamily.

### E. Polypeptide Delivery Methods

In some embodiments, one or more therapeutic proteins as described herein may be administered directly to a subject. For example, in certain aspects, the disclosure relates to a pharmaceutical composition comprising one or more therapeutic polypeptides selected from the group consisting of CHI3L1, an FGF protein, IL-2 ORF7, IL-2, an IL-17 protein and an IL-17 receptor; and a pharmaceutically acceptable carrier. In some embodiments, the one or more therapeutic polypeptides in the pharmaceutical composition are selected from the group consisting of CHI3L1, FGF-2, IL-2 ORF7, IL-2 and IL-17A. In certain aspects, the disclosure relates to a wound dressing comprising one or more therapeutic polypeptides selected from the group consisting of CHI3L1, an FGF protein, IL-2 ORF7, IL-2, an IL-17 protein and an IL-17 receptor. In some embodiments, the one or more therapeutic polypeptides in the wound dressing are selected from the group consisting of CHI3L1, FGF-2, IL-2 ORF7, IL-2 and IL-17A. In some embodiments, the recombinant RNA molecule encodes CHI3L1 and IL-2 ORF7. In some embodiments, the recombinant RNA molecule encodes CHI3L1 and IL-2.

In certain aspects, the disclosure relates to a method of preparing a wound dressing, the method comprising providing a composition comprising one or more therapeutic polypeptides selected from the group consisting of CHI3L1, an FGF protein, IL-2 ORF7, IL-2, an IL-17 protein and an IL-17 receptor, molding said composition into a desired shape, and lyophilizing said molded composition to yield a wound dressing. In some embodiments, the one or more therapeutic polypeptides used to prepare the wound dressing are selected from the group consisting of CHI3L1, FGF-2, IL-2 ORF7, IL-2 and IL-17A.

In certain aspects, the disclosure relates to a method of preparing a wound dressing, the method comprising providing an alginate solution comprising one or more therapeutic polypeptides selected from the group consisting of CHI3L1, an FGF protein, IL-2 ORF7, IL-2, an IL-17 protein and an IL-17 receptor, molding said alginate solution into a desired shape, inducing a cryo-organized structure by lyophilizing said molded alginate solution, and contacting said cryo-organized structure with a crosslinking agent to yield a wound dressing. In some embodiments, the one or more therapeutic polypeptides used to prepare the wound dressing are selected from the group consisting of CHI3L1, FGF-2, IL-2 ORF7, IL-2 and IL-17A.

In certain aspects, the disclosure relates to a method of treating a wound in a subject in need thereof, the method comprising administering a therapeutically effective amount of one or more therapeutic polypeptides selected from the group consisting of CHI3L1, an FGF protein, IL-2 ORF7, IL-2, an IL-17 protein and an IL-17 receptor to the wound, thereby treating the wound in the subject. In some embodiments, the one or more therapeutic polypeptides administered to the subject are selected from the group consisting of CHI3L1, FGF-2, IL-2 ORF7, IL-2 and IL-17A.

In certain aspects, the disclosure relates to a method of treating a wound in a subject in need thereof, the method comprising sequentially applying a therapeutically effective amount of a first therapeutic polypeptide and a second therapeutic polypeptide to the wound of the subject, wherein the first therapeutic polypeptide and the second therapeutic polypeptide are selected from the group consisting of CHI3L1, an FGF protein, IL-2 ORF7, IL-2, an IL-17 protein and an IL-17 receptor, and wherein the first therapeutic polypeptide and the second therapeutic polypeptide encode different polypeptides, thereby treating the wound in the subject. In some embodiments, the first therapeutic polypeptide and second therapeutic polypeptide administered to the subject are selected from the group consisting of CHI3L1, FGF-2, IL-2 ORF7, IL-2 and IL-17A.

### F. Carriers

The compositions, methods, and delivery systems (e.g., DNA, RNA, virus and polypeptide delivery systems) provided by the present disclosure may employ any suitable carrier. General considerations for carriers and delivery of pharmaceutical agents may be found, for example, in Delivery Technologies for Biopharmaceuticals: Peptides, Proteins, Nucleic Acids and Vaccines (Lene Jorgensen and Hanne Morck Nielson, Eds.) Wiley; 1st edition (Dec. 21, 2009); and Vargason et al. 2021. Nat Biomed Eng 5, 951-967.

Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the construct, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the construct), peptides (e.g., cell-penetrating peptides), materials (e.g., graphene oxide), single pure lipids (e.g., cholesterol), DNA origami (e.g., DNA tetrahedron).

In one embodiment, the compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for an agent described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the compositions and constructs described herein.

### G. Lipid Nanoparticles

In certain embodiments, the carrier is a lipid nanoparticle (LNP). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA or DNA) described herein includes,

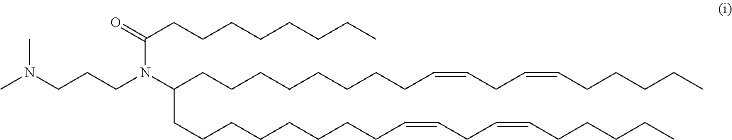 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., RNA or DNA) composition described herein to cells.

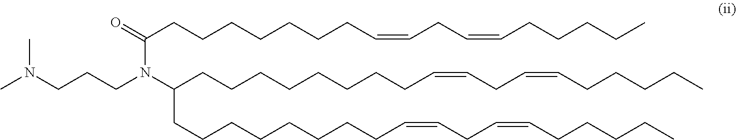 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., RNA or DNA) composition described herein to cells.

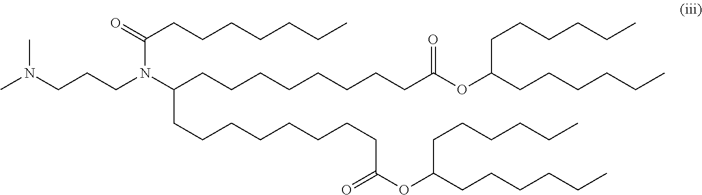 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., DNA or RNA) composition described herein to cells.

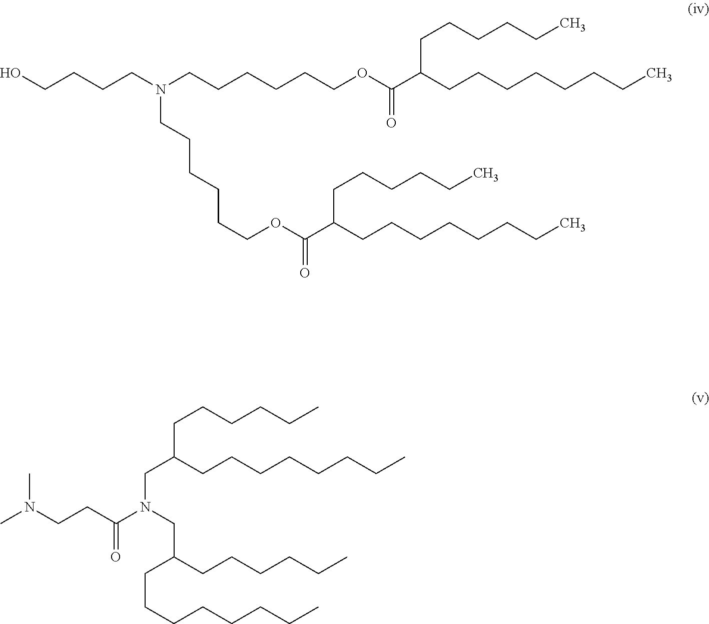

In some embodiments an LNP including Formula (v) is used to deliver a polyribonucleotide (e.g., DNA or RNA) composition described herein to cells.

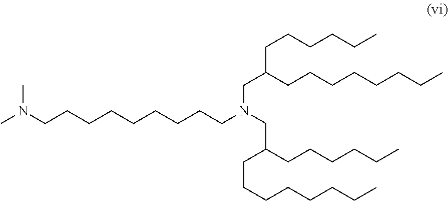 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., DNA or RNA) composition described herein to cells.

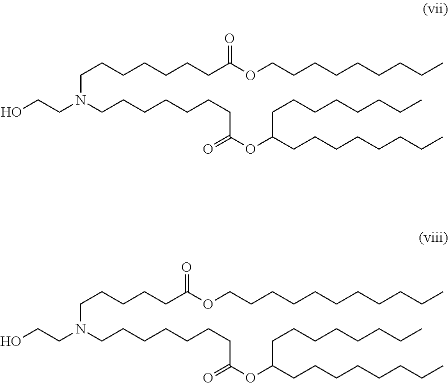 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., DNA or RNA) composition described herein to cells.

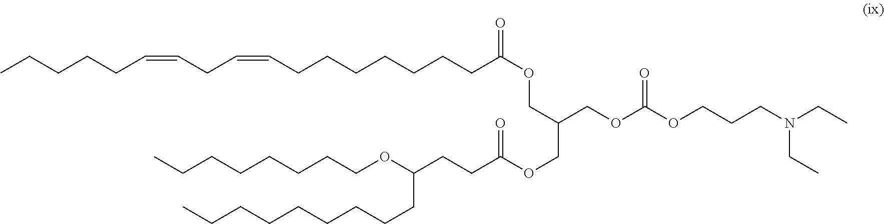 [SMILES: CCCCC/C=C\C/C=C\CCCCCCCC(=O)OCC(COC(=O)CCC(CCCCCCCCC)OCCCCCCCC)COC(=O)OCCCN(CC)CC]

In some embodiments an LNP including Formula (ix) is used to deliver a polyribonucleotide (e.g., DNA or RNA) composition described herein to cells.

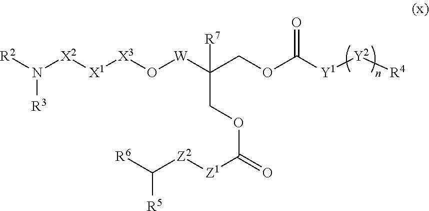

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

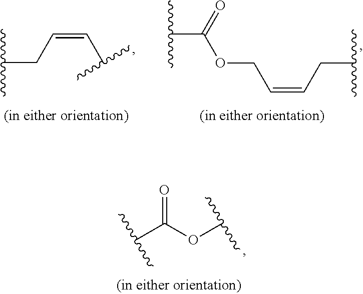 [SMILES: */C=C\C* ; *C/C=C\COC(*)=O ; *OC(*)=O]

n is 0 to 3, R⁴ is C1-15 alkyl, Z¹ is C1-6 alkylene or a direct bond,

### Z² is

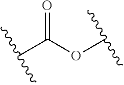 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

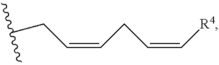

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., DNA or RNA) composition described herein to cells.

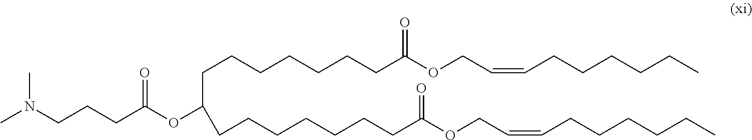 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., DNA or RNA) composition described herein to cells.

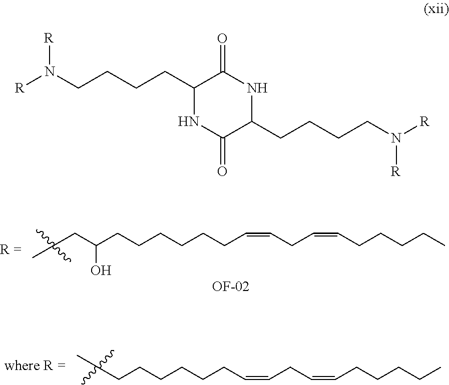

In some embodiments an LNP includes a compound of Formula (xiii) and a compound of Formula (xiv).

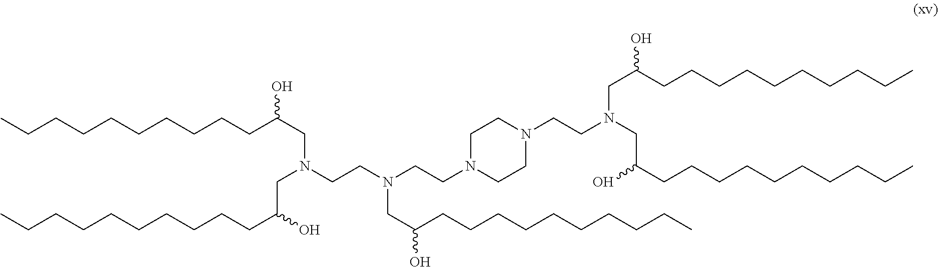 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., DNA or RNA) composition described herein to cells.

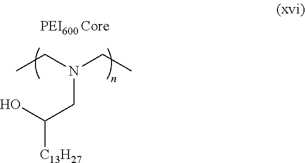

In some embodiments an LNP including a formulation of Formula (xvi) is used to deliver a polyribonucleotide (e.g., DNA or RNA) composition described herein to cells.

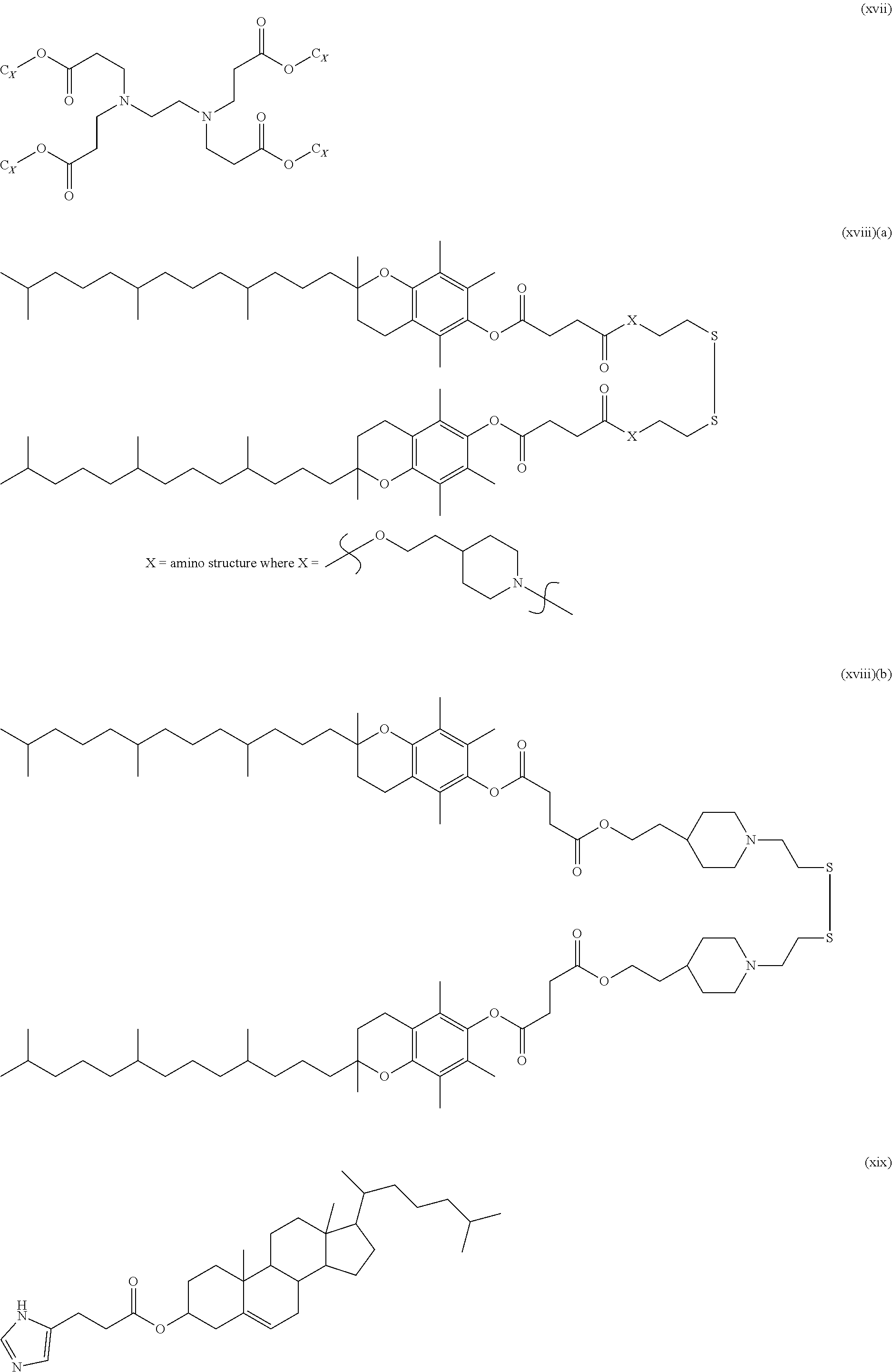

In some embodiments a lipid compound used to form lipid nanoparticles for the delivery of compositions described herein e.g nucleic acid (e.g., RNA or DNA) described herein is made by one of the following reactions:

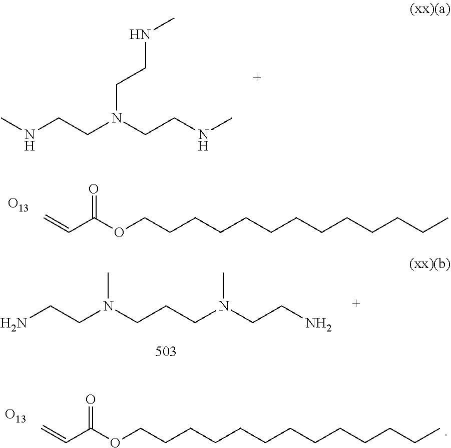

In some embodiments an LNP including Formula (xxi) is used to deliver a polyribonucleotide (e.g., DNA or RNA) composition described herein to cells. In some embodiments the LNP of Formula (xxi) is an LNP described by WO2021113777 (e.g., a lipid of Formula (1) such as a lipid of Table 1 of WO2021113777).

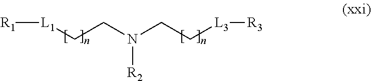

wherein

each n is independently an integer from 2-15; L₁ and L₃ are each independently —OC(O)—* or —C(O)O—*, wherein “*” indicates the attachment point to R₁ or R₃;

R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and

R₂ is selected from a group consisting of:

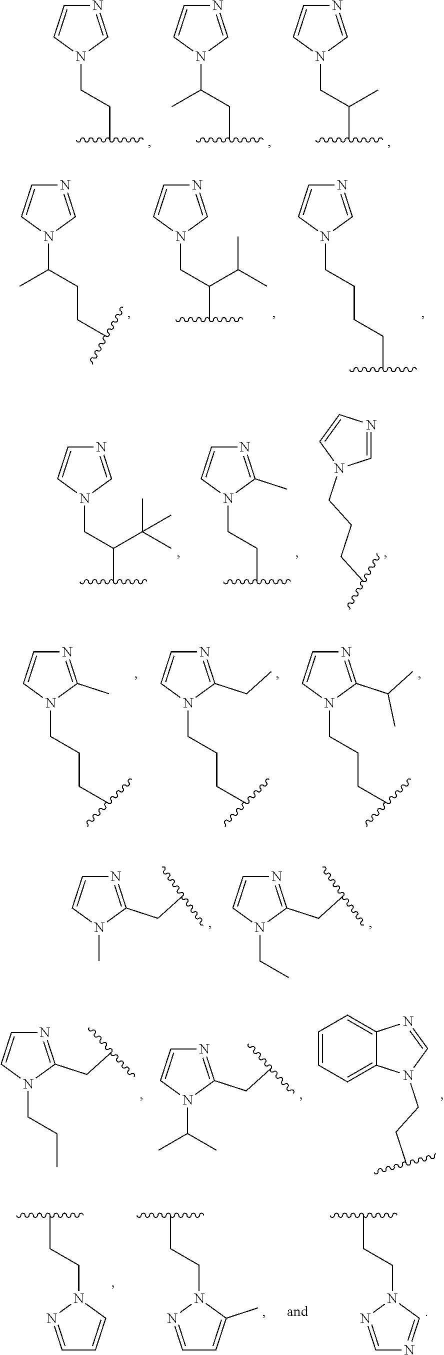

In some embodiments an LNP including Formula (xxii) is used to deliver a polyribonucleotide (e.g., DNA or RNA) composition described herein to cells. In some embodiments the LNP of Formula (xxii) is an LNP described by WO2021113777 (e.g., a lipid of Formula (2) such as a lipid of Table 2 of WO2021113777).

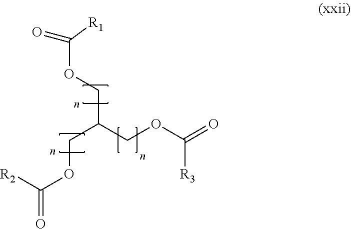

wherein

each n is independently an integer from 1-15;

R₁ and R₂ are each independently selected from a group consisting of:

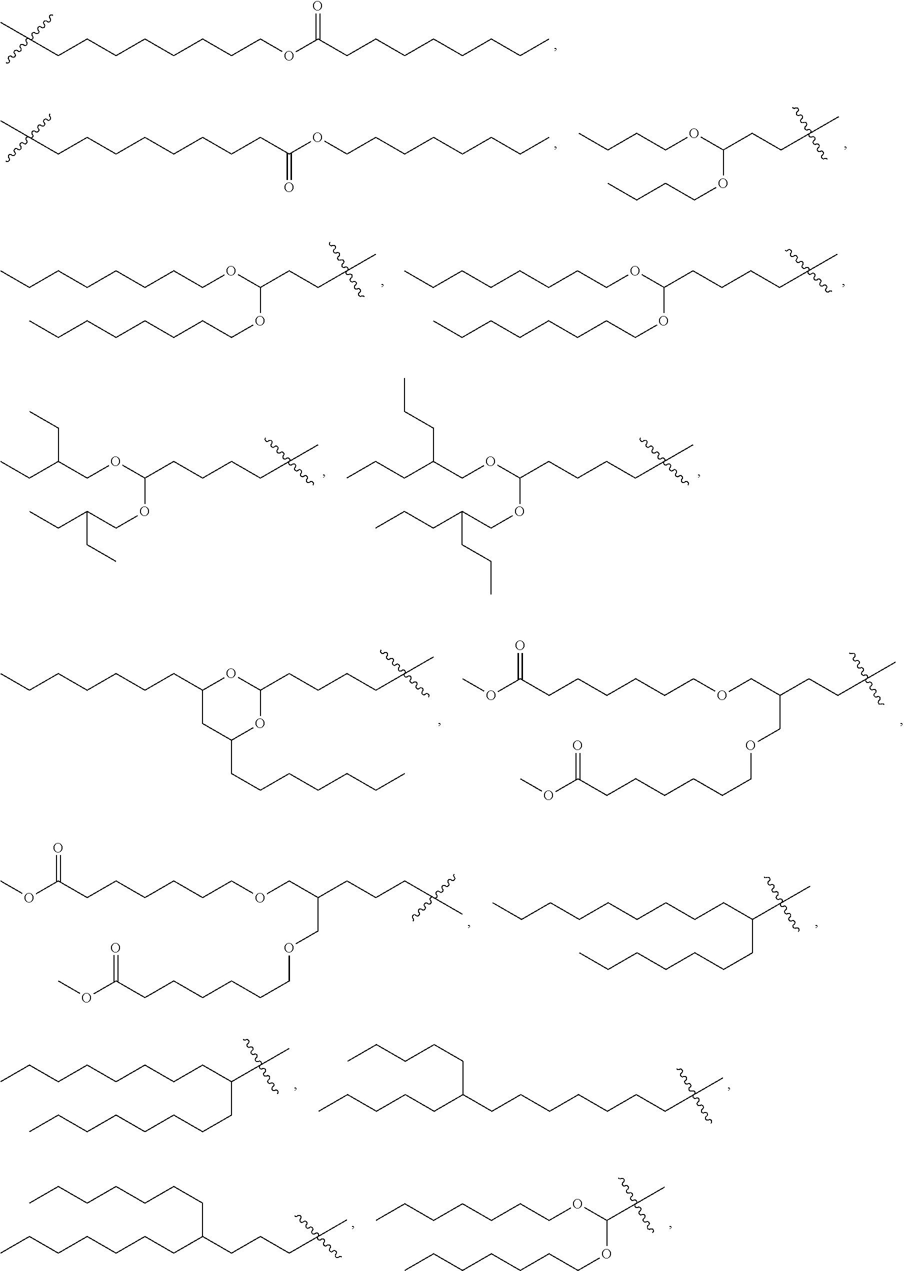

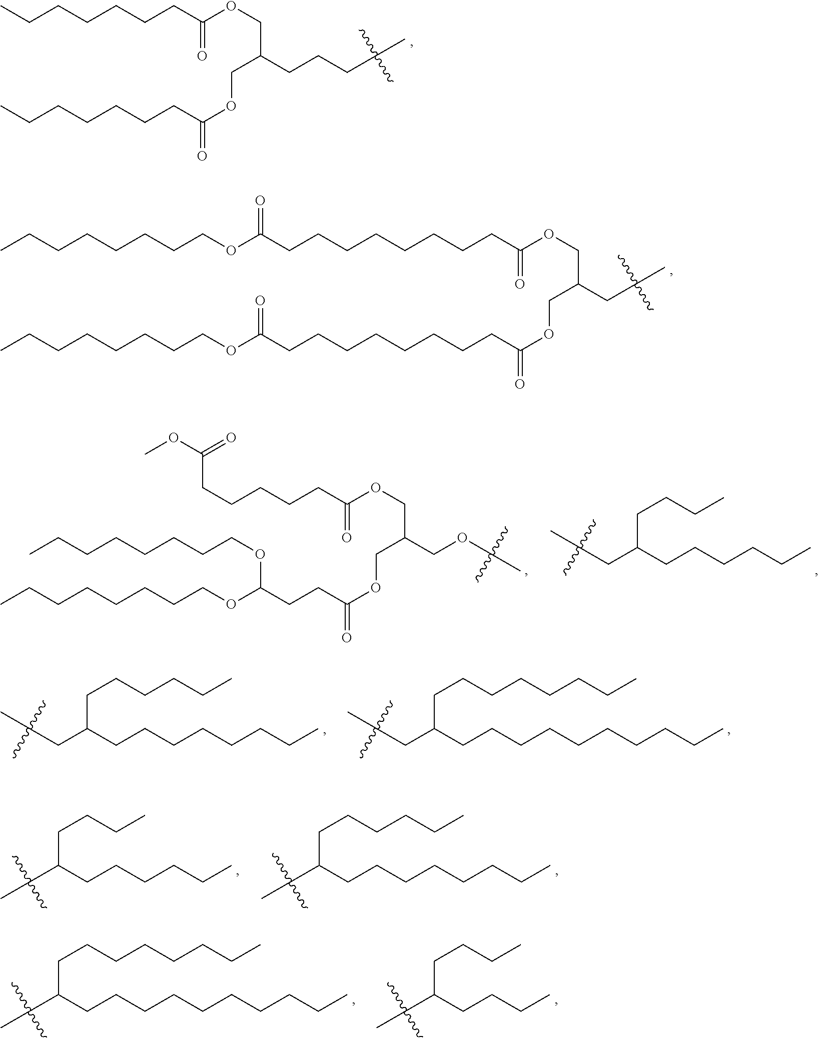

R₃ is selected from a group consisting of:

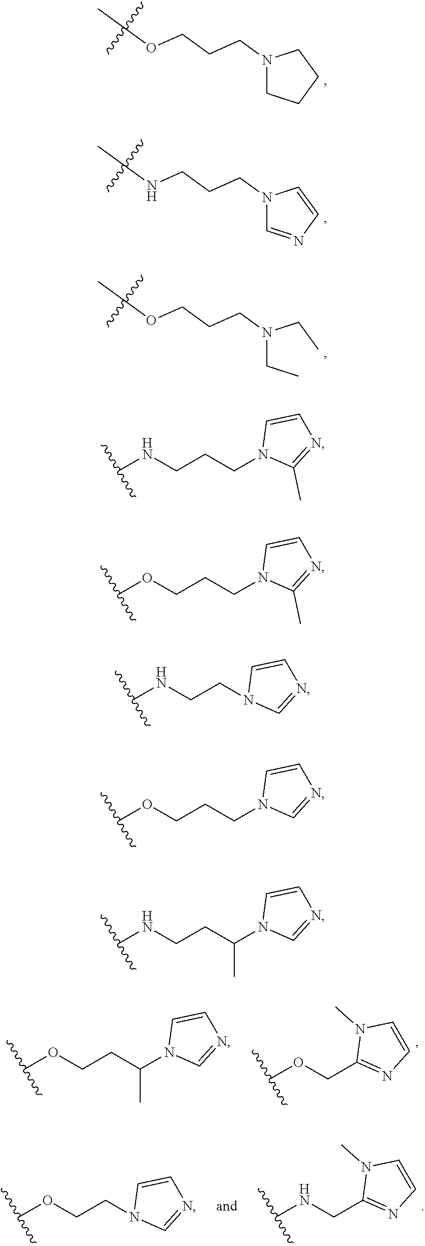

In some embodiments an LNP including Formula (xxiii) is used to deliver a polyribonucleotide (e.g., DNA or RNA) composition described herein to cells. In some embodiments the LNP of Formula (xxiii) is an LNP described by WO2021113777 (e.g., a lipid of Formula (3) such as a lipid of Table 3 of WO2021113777).

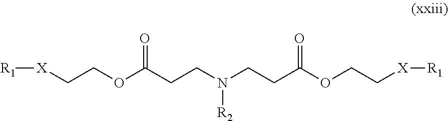

wherein

X is selected from —O—, —S—, or —OC(O)—*, wherein * indicates the attachment point R₁;

R₁ is selected from a group consisting of:

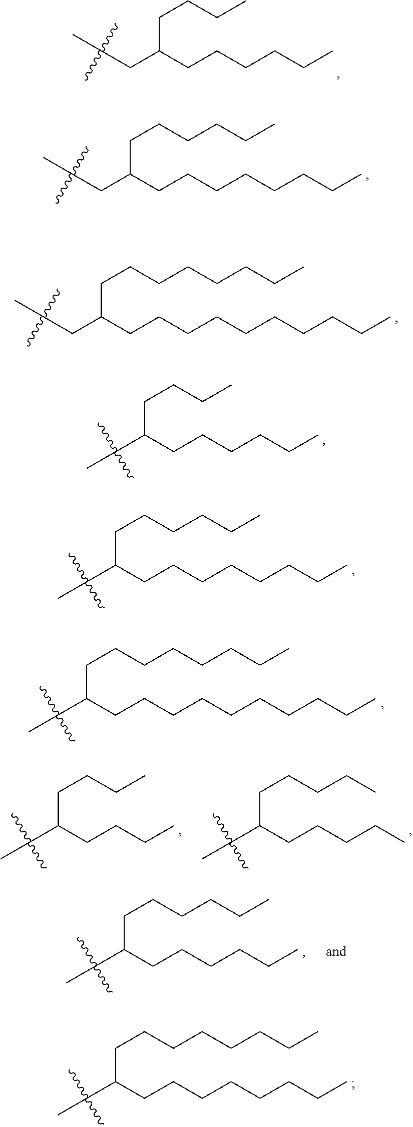

and R₂ is selected from a group consisting of:

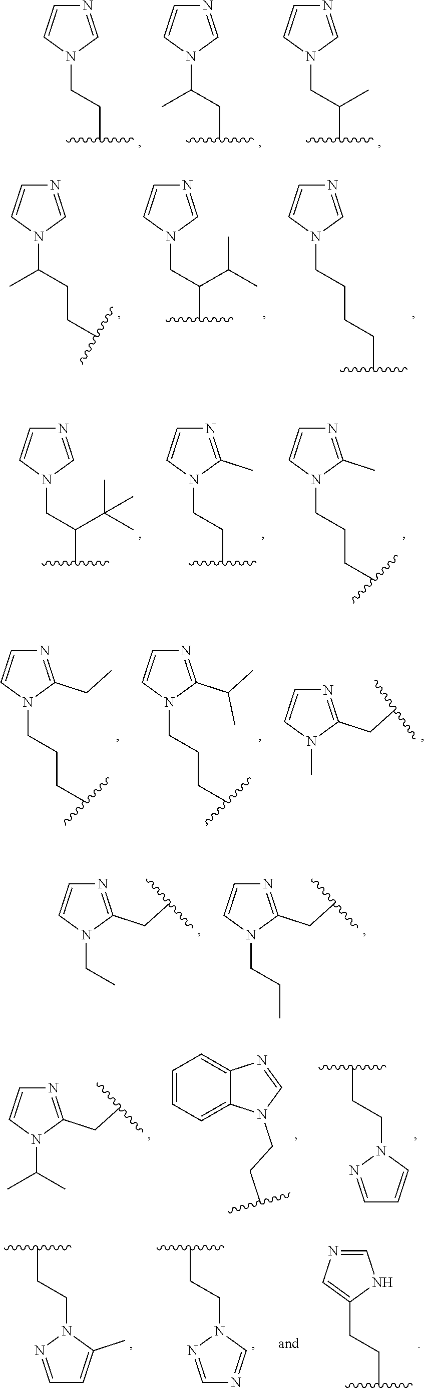

In some embodiments, a composition described herein (e.g., a nucleic acid (e.g., DNA or RNA) or a protein) is provided in an LNP that includes an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,l2Z)-3-((4,4-bis(octyloxy)butanoyl)oxy)-2-((((3-(diethylamino)propoxy)carbonyl)oxy)methyl)propyl octadeca-9,l2-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., DNA or RNA)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable.

In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-O13 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 lZ)-heptatriaconta-6,9,28,3 l-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (l3Z,l6Z)-A,A-dimethyl-3-nonyldocosa-l3, l6-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), l8-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-l-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

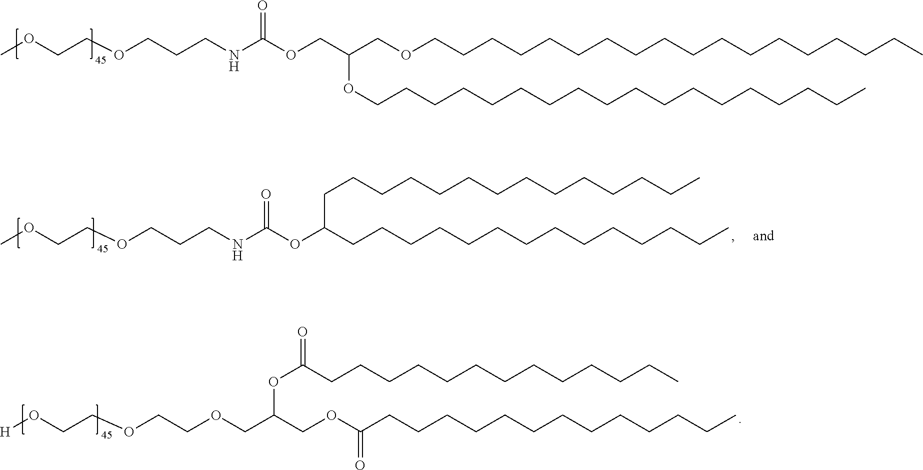

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,l2Z)-3-((4,4-bis(octyloxy)butanoyl)oxy)-2-((((3-(diethylamino)propoxy)carbonyl)oxy)methyl)propyl octadeca-9,l2-dienoate, also called 3-((4,4-bis(octyloxy)butanoyl)oxy)-2-((((3-(diethylamino)propoxy)carbonyl)oxy)methyl)propyl (9Z,l2Z)-octadeca-9,l2-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally include one or more coatings. In some embodiments, an LNP may be formulated in a capsule, film, or table having a coating. A capsule, film, or tablet including a composition described herein may have any useful size, tensile strength, hardness, or density.

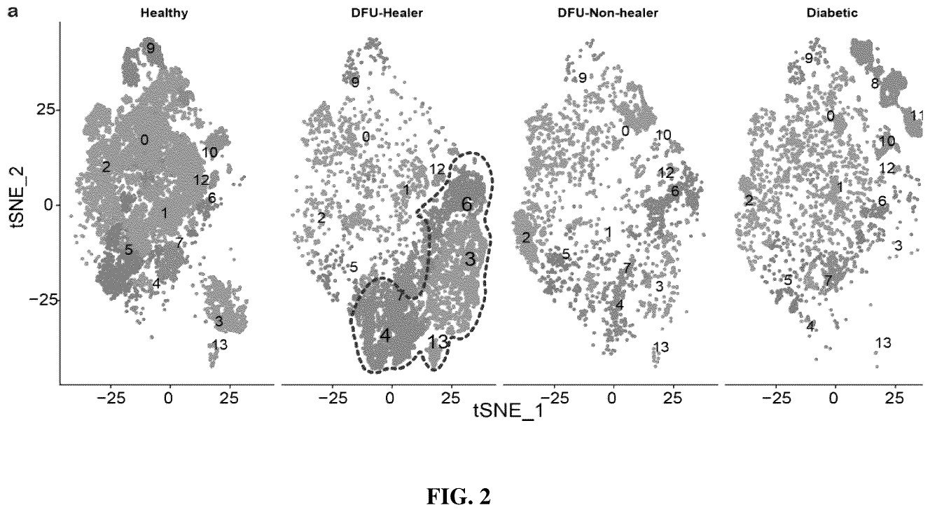

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of Figure 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of DNA or RNA compositions described herein.

Additional specific LNP formulations useful for delivery of nucleic acids (e.g., RNA, DNA) are described in U.S. Pat. Nos. 8,158,601 and 8,168,775, both incorporated by reference, which include formulations used in patisiran, sold under the name ONPATTRO.

Exemplary dosing of polyribonucleotide (e.g., DNA or RNA) LNP may include about 0.1, 0.25, 0.3, 0.5, 1, 2, 3, 4, 5, 6, 8, 10, or 100 mg/kg (RNA). Exemplary dosing of AAV including a polyribonucleotide (e.g., DNA or RNA) may include an MOI of about 10¹¹, 10¹², 10¹³, and 10¹⁴ vg/kg.

### Targeting of Lipid Nanoparticles

In some embodiments, the LNP is targeted to a particular cell type, e.g., a fibroblast, macrophage, keratinocyte, or endothelial cell. In some embodiments, the LNP is targeted to a particular cell by engineering the LNP to comprise a protein (e.g., an antibody or ligand) that is expressed on the surface of the cell. In some embodiments, the antibody or ligand that specifically binds the protein expressed on the surface of the cell is conjugated to a PEG-lipid in the LNP.

In some embodiments, the LNP is targeted to a fibroblast. For example, the LNP may be targeted to a fibroblast by engineering the LNP to comprise a protein (e.g., an antibody or ligand) that specifically binds to a protein expressed on the surface of a fibroblast, such as Fibroblast Activation Protein. Accordingly, in some embodiments, the LNP targeted to fibroblasts comprises an antibody or ligand that specifically binds Fibroblast Activation Protein. In some embodiments, the antibody or ligand that specifically binds the protein expressed on the surface of the fibroblast (e.g., Fibroblast Activation Protein) is conjugated to a PEG-lipid in the LNP.

In some embodiments, the LNP is targeted to a macrophage. For example, in some embodiments, the LNP is targeted to a macrophage by engineering the LNP to comprise a protein (e.g., an antibody or ligand) that specifically binds to a protein expressed on the surface of a macrophage, e.g., F4/80 antigen. F4/80 antigen, also referred to as ADGRE1, is encoded by the Adgre1 locus, and has been widely-used as a macrophage marker in mice. F4/80 antigen is a seven transmembrane G protein-coupled receptor with an extracellular domain containing repeated Epidermal Growth Factor (EGF)-like calcium binding domains. See Waddell et al., 2018, Front. Immunol., Vol. 9, doi.org/10.3389/fimmu.2018.02246. In some embodiments, the LNP targeted to macrophages comprises an antibody or ligand that specifically binds F4/80 antigen.

In some embodiments, the LNP is targeted to a keratinocyte. In some embodiments, the LNP is targeted to a keratinocyte by engineering the LNP to comprise a protein (e.g., an antibody or ligand) that specifically binds to a protein expressed on the surface of a keratinocyte, e.g., ketatinocyte-associated protein 2, or ketatinocyte-associated protein 3. Keratinocyte-associated protein 2 is a subunit of the oligosaccharyl transferase (OST) complex that catalyzes the initial transfer of a defined glycan (Glc₃Man₉GlcNAc₂ in eukaryotes) from the lipid carrier dolichol-pyrophosphate to an asparagine residue within an Asn-X-Ser/Thr consensus motif in nascent polypeptide chains, the first step in protein N-glycosylation. See Roboti et al., 2012, J. Cell Sci. 125:3474-3484. Keratinocyte-associated protein 3 plays a role in body weight regulation and demonstrates genotype- and sex-specific effects on food intake, adiposity, and insulin sensitivity. See Szalanczy et al., 2022, Front. Genet., doi.org/10.3389/fgene.2022.942574. In some embodiments, the LNP targeted to keratinocytes comprises an antibody or ligand that specifically binds ketatinocyte-associated protein 2, or ketatinocyte-associated protein 3.

In some embodiments, the LNP is targeted to an endothelial cell. In some embodiments, the LNP is targeted to an endothelial cell by engineering the LNP to comprise a protein (e.g., an antibody or ligand) that specifically binds to a protein expressed on the surface of an endothelial cell, e.g., Vascular Cell Adhesion Molecule 1 (VCAM-1). VCAM-1 is a cell surface sialoglycoprotein expressed by cytokine-activated endothelial cells. See Su et al., 2022, J Immunol Res., doi: 10.1155/2022/6137219. In some embodiments, the LNP targeted to endothelial cells comprises an antibody or ligand that specifically binds VCAM-1.

### IV. Methods of Treating Wounds

In certain aspects, the present disclosure relates to methods of treating a wound (e.g., a diabetic wound) in a subject by increasing expression of one or more therapeutic polypeptides as described herein (e.g., a protein selected from chitinase-3-like protein 1 (CHI3L1), fibroblast growth factor 2 (FGF-2), interleukin-2 ORF7 (IL-2 ORF7), interleukin-2 and interleukin-17A (IL-17A) in the wound by administering one or more recombinant nucleic acid molecules (e.g., RNA molecules) encoding the one or more therapeutic proteins to the subject.

For example, in certain aspects, the disclosure relates to a method of increasing expression of one or more polypeptides in a wound in a subject, wherein the one or more polypeptides is selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL-17 receptor, the method comprising administering a recombinant nucleic acid molecule (e.g., RNA molecule) encoding the one or more polypeptides to a wound (e.g., a diabetic wound) of a subject in an amount sufficient to increase expression of the one or more polypeptides.

In certain aspects, the disclosure relates to a method of increasing expression of one or more polypeptides in a wound in a subject, wherein the one or more polypeptides is selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), the method comprising administering a recombinant nucleic acid molecule (e.g., RNA molecule) encoding the one or more polypeptides to a wound (e.g., a diabetic wound) of a subject in an amount sufficient to increase expression of the one or more polypeptides.

In some embodiments, expression of chitinase-3-like protein 1 (CHI3L1), a fibroblast growth factor (FGF), interleukin-2 ORF7 (IL-2 ORF7), interleukin-2 (IL-2) an interleukin-17 (IL-17) protein, and/or an IL-17 receptor is increased in mesenchymal cells in a wound (e.g., a diabetic wound) of the subject. In some embodiments, expression of chitinase-3-like protein 1 (CHI3L1), a fibroblast growth factor (FGF), interleukin-2 ORF7 (IL-2 ORF7), interleukin-2 (IL-2) an interleukin-17 (IL-17) protein, and/or an IL-17 receptor is increased in macrophages in a wound (e.g., a diabetic wound) of the subject.

In certain aspects, the disclosure relates to a method of treating a wound in a subject in need thereof, the method comprising administering a therapeutically effective amount of a recombinant RNA molecule encoding one or more polypeptides selected from the group consisting of chitinase-3-like protein 1 (CHI3L1), a fibroblast growth factor (FGF), interleukin-2 ORF7 (IL-2 ORF7), interleukin-2 (IL-2) an interleukin-17 (IL-17) protein, and an IL-17 receptor to the wound, thereby treating the wound in the subject.

In certain aspects, the disclosure relates to a method of treating a wound in a subject in need thereof, the method comprising administering a therapeutically effective amount of a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A) to the wound, thereby treating the wound in the subject.

In some embodiments, the wound is a skin/cutaneous lesion (e.g., a chronic skin lesion) or a mucous membrane lesion. Exemplary skin lesions include but are not limited to burns, eczema, psoriasis, disease wounds, acne, actinic keratosis, allergic/contact dermatitis, boils (infections that develop on hair follicles), bullae (fluid-filled sacs or lesions that appear when fluid is trapped under a layer of skin), cellulitis, chemical burns, cherry angiomas, chicken pox, cold sores, corns, calluses, cysts, dyshidrotic eczema (itchy blisters on the palms of hands and/or soles of feet), erysipelas (bacterial infections that affect the skin's upper layers), frostbite, genital herpes, gout, impetigo, insect sting allergies (e.g., mosquito bites, wasp stings), keloids (smooth, hard growths that form when scar tissue grows excessively), keratosis pilaris, lipomas, molluscum contagiosum (skin infections caused by the molluscum virus), methicillin-resistant Staphylococcus (Staph) infections, neurofibromas, nodules (abnormal growths that form under the skin, often filled with inflamed tissue or fluid), pemphigoid (a rare autoimmune disorder that more often affects the elderly), pemphigus vulgarus (an autoimmune disease that leads to painful blisters on skin and mucous membranes), porphyrias, scabies, scarlet fever, sebaceous cysts, seborrheic keratosis, shingles, skin cancer, and warts.

In some embodiments, the wound is a foot ulceration, e.g., a diabetic foot ulceration (DFU). Foot ulceration is a major problem in diabetic patients as more than 15% are expected to develop a diabetic foot ulceration (DFU) within their lifetime. See Theocharidis et al., 2022, Nature communications 13(1):181, which is incorporated by reference herein in its entirety. DFU significantly impairs quality of life, leads to prolonged hospitalization and results in more than 70,000 lower extremity amputations per year in the USA alone. See Theocharidis et al., cited above; and Bhat et al., 2021, Trends Mol Med. 2021; 27(9):923-4. With the expected increase of diabetes incidence, DFUs will represent an even bigger burden for the health system of both developed and developing countries and may prove to be one of the most costly diabetes complications. There are currently four products available for the treatment of DFU: a recombinant growth factor (rhPDGF-BB, becaplermin; Vital Statistics. Alexandria, VA: 1996), two bioengineered skin substitutes, Apligraf (Geiss et al., 2018, Diabetes Care. Epub 2018/11/10. doi: 10.2337/dc18-1380) and Dermagraft (Boulton et al., 2005, Lancet 366(9498):1719-24), and Integra (Wieman et al., 1998, Diabetes Care 21(5):822-7), an acellular, bilayer matrix. These products were all developed in the 1990s and are characterized by moderate efficacy, at best achieving complete wound healing in about 50% of patients (Veves et al., 2001, Diabetes Care 24(2):290-5; and Marston et al., 2003, Diabetes Care 26(6):1701-5).

In some embodiments, the size of a wound of the subject is reduced compared to the size of the wound prior to contacting the subject with a composition, e.g., a pharmaceutical composition or wound dressing, described herein. For example, in some embodiments, the size of the wound is reduced by at least 1.5-fold, e.g., at least 1.5-fold, 2-fold, 3-fold, 4-fold, 5-fold, 10-fold, 15-fold, 20-fold, 30-fold, 40-fold, 50-fold relative to the size before treatment with the composition. In some embodiments, the size of a wound is a perimeter or area of the wound. In some embodiments, administration of the composition, e.g., pharmaceutical composition or wound dressing, results in a complete closure of a wound in the subject. For example, in some embodiments, complete closure of the wound occurs within 6 months, e.g., within 6 months, 5 months, 4 months, 3 months, 2 months, 1 month, 5 weeks, 4 weeks, 3 weeks, 2 weeks, 1 week, 7 days, 6 days, 5 days, 4 days, 3 days, 2 days, 1 day, or less, after administration of the composition.

In some embodiments, the compositions described herein (e.g., pharmaceutical compositions and wound dressings) are effective in treating or alleviating a symptom of a skin lesion, e.g., a chronic skin lesion. Exemplary skin lesions are described above. For example, in some embodiments, the compositions described herein are effective in treating or alleviating a symptom of eczema, psoriasis, frostbite, bacterial infections, or burns.

In some embodiments, the composition (e.g., pharmaceutical composition or wound dressing) directly contacts an injured, damaged, or diseased tissue, e.g., at the site of a cutaneous/mucous membrane injury, damage, or disease (such as a skin/mucous membrane lesion or disease described above). In some cases, the composition covers the site of a cutaneous/mucous membrane injury and extends to and/or overlaps onto healthy unaffected tissue, e.g., healthy unaffected skin or mucous membrane.

In some embodiments, the compositions described herein (e.g., pharmaceutical compositions and wound dressings) are effective in reducing pain in a subject. In some examples, the compositions of the invention are effective in reducing pain in a subject by at least 5%, e.g., at least 5%, 10%, 15%, 20%, 25%, 30%, 40%, 50%, 60%, 70%, 80%, 90%, 95%, or greater, compared to the level of pain prior to administration of the composition or device/bandage comprising the composition. In some cases, pain, e.g., at the site of administration, is eliminated after administration of the composition.

In some embodiments, the subject has diabetes. For example, in some embodiments, the subject has a diabetic wound, e.g., an ischemic wound. In some embodiments, the ischemic wound is a neuroischemic wound.

For example, in certain aspects the disclosure relates to a method of treating a diabetic ulcer wound in a subject in need thereof, the method comprising applying a wound dressing to the diabetic ulcer wound, wherein the wound dressing comprises a therapeutically effective amount of a recombinant RNA molecule encoding one or more polypeptides selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL17 receptor, thereby treating the diabetic ulcer wound. In embodiments, the recombinant RNA molecule is comprised within a lipid nanoparticle (LNP).

In certain aspects the disclosure relates to a method of treating a diabetic ulcer wound in a subject in need thereof, the method comprising applying a wound dressing to the diabetic ulcer wound, wherein the wound dressing comprises a therapeutically effective amount of a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A), thereby treating the diabetic ulcer wound. In embodiments, the recombinant RNA molecule is comprised within a lipid nanoparticle (LNP),

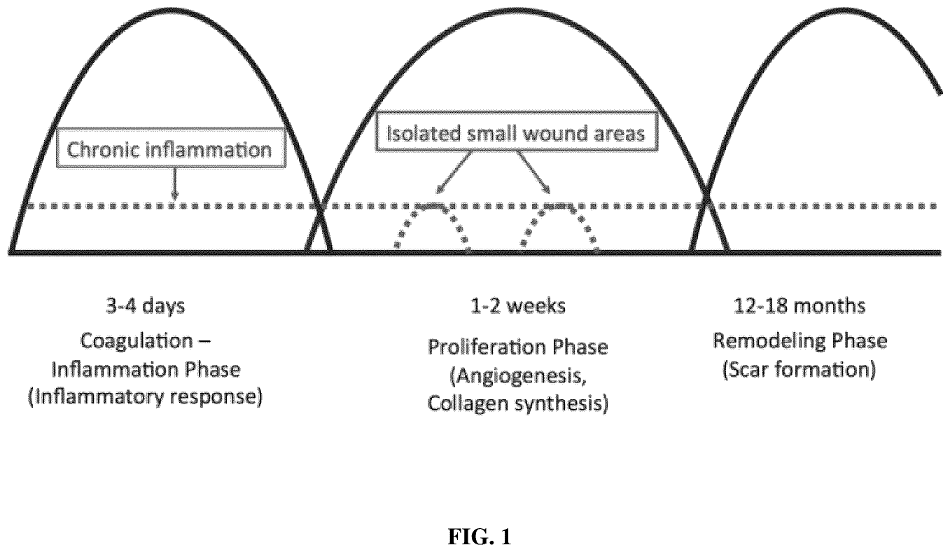

Although there is some overlap, acute normal wound healing can be divided into three phases: coagulation-inflammation, proliferation and remodeling (Falanga et al., 2022, Nat Rev Dis Primers. 8(1):50.). The coagulation-inflammation phase is characterized by rapid blood clot formation followed by intense infiltration by inflammatory cells, such as neutrophils and macrophages; the proliferation phase, marked by angiogenesis, deposition of extracellular matrix (ECM) and keratinocyte migration; and the remodeling phase, which includes remodeling of the ECM and the development of a permanent scar (Singer et al., 1999, The New England journal of medicine 341(10):738-46; and Santoro et al., 2005, Exp Cell Res. 304(1):274-86). Chronic wounds, like DFU, lack this linear progression from one phase to the next and are mainly characterized by the persistence of the inflammatory phase, although progression to the proliferation phase may be sporadically present in certain wound areas (see Figure 1, Singer, cited above, and Eming et al., 2014, Science translational medicine 6(265): 265).

In certain aspects, the disclosure relates to a method of treating a wound in a subject in need thereof, the method comprising sequentially applying a therapeutically effective amount of a first recombinant nucleic acid molecule (e.g., RNA molecule) and a second recombinant nucleic acid molecule (e.g., RNA molecule) to the wound of the subject, wherein the first recombinant nucleic acid molecule and the second recombinant nucleic acid molecule each encode one or more polypeptides selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL-17 receptor, and wherein the first recombinant nucleic acid molecule and the second recombinant nucleic acid molecule encode different proteins, thereby treating the wound in the subject.

In certain aspects, the disclosure relates to a method of treating a wound in a subject in need thereof, the method comprising sequentially applying a therapeutically effective amount of a first recombinant nucleic acid molecule (e.g., RNA molecule) and a second recombinant nucleic acid molecule (e.g., RNA molecule) to the wound of the subject, wherein the first recombinant nucleic acid molecule and the second recombinant nucleic acid molecule each encode one or more polypeptides selected from the group consisting of chitinase-3-like protein 1 (CHI3L1), fibroblast growth factor (FGF-2), interleukin-2 ORF7 (IL-2 ORF7), interleukin-2 (IL-2), and interleukin-17A (IL-17A), and wherein the first recombinant nucleic acid molecule and the second recombinant nucleic acid molecule encode different proteins, thereby treating the wound in the subject. In some embodiments, the first recombinant nucleic acid molecule is applied to the wound before the second recombinant nucleic acid molecule is applied to the wound. For example, in some embodiments, the first recombinant nucleic acid molecule is applied to the wound 1, 2, 3, 4, 5, 6, 7, 8, 9, 10, 11, 12, 13 or 14 days before the second recombinant nucleic acid molecule is applied to the wound. In some embodiments, the first recombinant nucleic acid molecule is applied to the wound during the inflammation phase of wound healing. In some embodiments, the second recombinant nucleic acid molecule is applied to the wound in a phase of wound healing after the inflammation phase.

In some embodiments, the first recombinant nucleic acid molecule encodes CHI3L1, and the second recombinant nucleic acid molecule encodes a polypeptide selected from the group consisting of a fibroblast growth factor (FGF), interleukin-2 ORF7 (IL-2 ORF7), interleukin-2 (IL-2), an interleukin-17 (IL-17) protein, and an IL-17 receptor. In some embodiments, the first recombinant nucleic acid molecule encodes CHI3L1, and the second recombinant nucleic acid molecule encodes a polypeptide selected from the group consisting of a fibroblast growth factor (FGF), interleukin-2 ORF7 (IL-2 ORF7), interleukin-2 (IL-2), and an interleukin-17 (IL-17) protein. In some embodiments, the first recombinant nucleic acid molecule encodes CHI3L1, and the second recombinant nucleic acid molecule encodes IL-2. In some embodiments, the first recombinant nucleic acid molecule encodes CHI3L1, and the second recombinant nucleic acid molecule encodes IL-2 ORF7. In some embodiments, the first recombinant nucleic acid molecule encodes CHI3L1, and the second recombinant nucleic acid molecule encodes FGF. In some embodiments, the first recombinant nucleic acid molecule encodes CHI3L1, and the second recombinant nucleic acid molecule encodes IL-17 protein. In some embodiments, the first recombinant nucleic acid molecule encodes CHI3L1, and the second recombinant nucleic acid molecule encodes IL-17 receptor.

In some embodiments, the first recombinant nucleic acid molecule and the second recombinant nucleic acid molecule are comprised within different compositions, e.g., within different wound dressings, and said compositions are sequentially applied to the subject. For example, in some embodiments, the wound dressing comprising the first recombinant nucleic acid molecule is removed from the wound before the wound dressing comprising the second recombinant nucleic acid molecule is applied to the wound.

In some embodiments, the first recombinant nucleic acid molecule (e.g., RNA molecule) and the second recombinant nucleic acid molecule (e.g., RNA molecule) are comprised within the same composition, e.g., within the same wound dressing. In these embodiments, sequential administration may be achieved by formulating the first recombinant nucleic acid molecule and the second recombinant nucleic acid molecule such that they are released from the composition (e.g., the wound dressing) to the subject at different times. For example, in some embodiments, sequential delivery occurs when the first recombinant nucleic acid molecule (e.g., RNA molecule) is comprised within highly charged LNPs, and the second recombinant nucleic acid molecule (e.g., RNA molecule) is comprised within lowly charged LNPs. The recombinant nucleic acid molecule comprised within lowly charged LNPs releases first, followed by the recombinant nucleic acid molecule comprised within highly charged LNPs. In some embodiments, sequential administration of the first recombinant nucleic acid molecule and second recombinant nucleic acid molecule may be achieved by using LNPs with different surface charges, e.g., by coating the LNPs with different types or concentrations of polycations. For example, the electrostatic interaction between the polycation coated LNPs and the wound dressing (e.g., an alginate dressing) can be altered by varying the polycation concentration or by employing different coating materials, such as bPEI, chitosan or gelatin.

In some embodiments of the foregoing methods, migration of endothelial cells is increased in the wound. In some embodiments of the foregoing methods, migration of dermal fibroblasts is increased in the wound.

In embodiments of the foregoing methods, the recombinant RNA molecule(s) may be administered by any route of administration described herein. In some embodiments, the recombinant RNA molecule(s) is administered topically, e.g., to the site of the wound. In some embodiments, the recombinant RNA molecule(s) is administered by applying a wound dressing, as described herein, to the wound, wherein the wound dressing comprises the recombinant RNA molecule(s).

In embodiments of the foregoing methods, the recombinant RNA molecule(s) is administered in a therapeutically effective amount.

In some embodiments, the subject is human. In some embodiments, the subject is a non-human mammal, e.g., a mouse, rat, dog, rabbit, pig, or monkey.

### V. Pharmaceutical Compositions and Modes of Administration

In certain aspects, the present disclosure relates to a pharmaceutical composition comprising a recombinant nucleic acid molecule (e.g., a recombinant RNA molecule), vector (e.g. an engineered virus, plasmid or transposon), therapeutic protein, or cell as described herein, and a pharmaceutically acceptable carrier.

In certain aspects, the present disclosure relates to a pharmaceutical composition comprising: (a) two or more recombinant RNA molecules each encoding a different polypeptide, wherein each of the polypeptides is selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL-17 receptor; and (b) a pharmaceutically acceptable carrier.

In certain aspects, the present disclosure relates to a pharmaceutical composition comprising: (a) two or more recombinant RNA molecules each encoding a different polypeptide, wherein each of the polypeptides is selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A); and (b) a pharmaceutically acceptable carrier.

The pharmaceutical compositions described herein may be administered to a subject in any suitable formulation. These include, for example, liquid, semi-solid, and solid dosage forms. The preferred form depends on the intended mode of administration and therapeutic application.

In some embodiments the pharmaceutical composition is formulated for topical administration. In certain embodiments, the pharmaceutical composition is formulated for parenteral administration, including topical administration, and intravenous, intraperitoneal, intramuscular, and subcutaneous, injections. In some embodiments, the pharmaceutical composition is formulated for intravenous administration. In some embodiments the pharmaceutical composition is formulated for oral administration.

Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients (i.e., one or more recombinant nucleic acid molecules as described herein) are contained in an effective amount to achieve its intended purpose, e.g., promoting wound healing (e.g., diabetic wound healing), or increasing expression of one or more therapeutic proteins. In some embodiments, the pharmaceutical composition comprises a therapeutically effective amount of one or more recombinant nucleic acid molecules described herein. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the composition is delivered topically. In certain embodiments, the pharmaceutical composition is delivered orally. In certain embodiments, the composition is administered parenterally. In certain embodiments, the composition is delivered by injection or infusion. In certain embodiments, the composition is delivered by inhalation. In one embodiment, the compositions provided herein may be administered by injecting directly to a wound. In some embodiments, the compositions may be administered by intravenous injection or intravenous infusion. In certain embodiments, administration is systemic. In certain embodiments, administration is local.

### VI. Wound Dressings

In certain aspects, the disclosure relates to a wound dressing comprising a recombinant nucleic acid molecule (e.g., RNA molecule) encoding one or more polypeptides selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL-17 receptor. In some embodiments, the recombinant nucleic acid molecule (e.g., RNA molecule) comprises at least one chemical modification.

In certain aspects, the disclosure relates to a wound dressing comprising a recombinant nucleic acid molecule (e.g., RNA molecule) encoding one or more polypeptides selected from the group consisting of chitinase-3-like protein 1 (CHI3L1), fibroblast growth factor 2 (FGF-2), interleukin-2 ORF7 (IL-2 ORF7), interleukin-2 (IL-2) and interleukin-17A (IL-17A). In some embodiments, the recombinant nucleic acid molecule (e.g., RNA molecule) comprises at least one chemical modification.

The dimensions of the wound dressing are those typical for topically applied bandages, i.e., the of the wound dressing corresponds to the wound to be treated. In some embodiments, the thickness of the wound dressing is 0.1 mm-10 mm, e.g., 1 mm-5 mm or 1 mm-2 mm, Release of the recombinant nucleic acid molecule (e.g., RNA molecule) from the wound dressing depends on the affinity of the recombinant nucleic acid molecule for the network structure of the wound dressing, e.g., alginate, as well as diffusion. Thus, a thinner gel, e.g., 1 mm-5 mm in thickness, permits more rapid release of the recombinant nucleic acid molecule (e.g., RNA molecule) to a tissue than a thicker dressing. A thickness of 1 mm-2 mm is compatible with the, customary clinical schedule of changing would dressings. For slower release, the bandage is optionally fabricated at a greater thickness, thereby retarding drug release due to the greater distance the drug must travel to exit the dressing.

The width and length of the wound dressing vary depending on the size of the wound or the use. For example, the width and/or length of the wound dressing comprises 0.5 cm-12 cm, e.g. 0.5 cm-6 cm, 0.5 cm-3 cm, 0.5 cm-2 cm, 1 cm-12 cm, 2 cm-12 cm, 3 cm-12 cm, 6 cm-12 cm, 8 cm-12 cm, 10 cm-12 cm, 2 cm-10 cm, 2 cm-8 cm, 2 cm-6 cm, or 4 cm-8 cm. For example, the surface area of the bandage or wound dressing comprises 0.2 cm²-144 cm², e.g., 0.2 m²-100 cm², 0.2 cm²-60 cm, 0.2 cm²-30 cm², 0.2 cm²-10 cm², 0.2 cm²-4 cm², cm²-100 cm², 1 cm²-60 cm², 1 cm²-30 cm², 1 cm²-10 cm², 5 cm²-144 cm², 5 cm²-100 cm², 5 cm²-60 cm², 5 cm²-30 cm², 25 cm²-144 cm², 25 cm²-100 cm², 100 cm²-144 cm², 25 cm²-100 cm², or 40 cm²-80 cm².

In some embodiments, the wound dressing comprises 0.1, 0,5, 1, 2, 3, 4, 5, 6, 7, 8, 9, 10, 11, 12, 13, 14, 15, 16, 17, 18, 19, 20, 21, 22, 23, 24 or 25 μg of the recombinant nucleic acid molecule (e.g., RNA molecule). In some embodiments, the wound dressing comprises at least 0.1, 0.5, 1, 3, 4, 5, 6, 7, 8, 9, 10, 11, 12, 13, 14, 15, 16, 17, 18, 19, 20, 21, 22, 23, 24 or 25 μg of the recombinant nucleic acid molecule (e.g., RNA molecule). In some embodiments, the wound dressing comprises less than 0.1, 0.5, 1, 2, 3, 4, 5, 6, 7, 8, 9, 10, 11, 12, 13, 14, 15, 16, 17, 18, 19, 20, 21, 22, 23, 24 or 25 μg of the recombinant nucleic acid molecule (e.g., RNA molecule), Any of these values may be used to define a range for the amount of the recombinant nucleic acid molecule in the wound dressing. For example, in some embodiments, the wound dressing comprises 0.1-25, 0.5-10 or 1-10 μg of the recombinant nucleic acid molecule (e.g., RNA molecule), In a particular embodiment, the wound dressing comprises 4 μg of the recombinant nucleic acid molecule (e.g., RNA molecule).

In some embodiments, the concentration of the recombinant nucleic acid molecule (e.g., RNA molecule) in the wound dressing is 1, 10, 20, 30, 40, 50, 60, 70, 80, 90, 100, 200, 300, 400, 500, 600, 700, 800, 900 or 1000 μg/ml. In some embodiments. the concentration of the recombinant nucleic acid molecule (e.g., RNA molecule) in the wound dressing is at least 1, 10, 20, 30, 40, 50, 60, 70, 80, 90, 100, 200, 300, 400, 500, 600, 700, 800, 900 or 1000 μg/mL. In some embodiments, the concentration of the recombinant nucleic acid molecule (e.g., RNA molecule in the wound dressing is less than 1, 10, 20, 30, 40, 50, 60, 70, 80, 90, 100, 200, 300, 400, 500, 600, 700, 800, 900 or 1000 μg/ml. Any of these values may be used to define a range for the concentration of the recombinant nucleic acid molecule in the wound dressing. For example, in some embodiments, the wound dressing comprises 1-1000, 10-200, or 50-200 μg/ml of the recombinant nucleic acid molecule (e.g., RNA molecule), In a particular embodiment, the wound dressing comprises 100 μg/mil of the recombinant nucleic acid molecule (e.g., RNA molecule).

In some embodiments, the wound dressing maintains the moisture of the wound dressing after topical administration onto a subject. For example, in some embodiments, the wound dressing loses less than 60%, e.g., less than 60%, 50%, 40%, 30% 20%, 15%, 10%, 5% %, or less, of the water content after administration (e.g., at least 1 hour, e.g., at least 1, 2, 3, 4, 5, 6, 7,8, 9, 10, 12, or 24 hours, 1, 2, 3, 4, 5, 6, or 7 days, 1, 2, 3, 4, or 5 weeks, or 1, 2, 3, 4, 5, 6, 7, 8, 9, or 10 months, or more after administration) compared to prior to administration. For example, in some embodiments, the wound dressing loses less than 60%, e.g., less than 60%, 50%, 40%, 30%, 20%, 15%, 10%, 5%, 2%, 1%, or less, of its weight or mass after administration (e.g., at least 1 hour, e.g., at least 1, 2, 3, 4, 5, 6, 7, 8, 9, 10, 12, or 24 hours, 1, 2, 3, 4, 5, 6, or 7 days, 1, 2, 3, 4, or 5 weeks, or 1, 2, 3, 4, 5, 6, 7, 8, 9, or 10 months, or more after administration) compared to prior to administration.

In certain aspects, the disclosure relates to a method of preparing a wound dressing, the method comprising providing a composition comprising a recombinant nucleic acid molecule (e.g., RNA molecule), LNP, or pharmaceutical composition as described herein, molding said composition into a desired shape, and lyophilizing said molded composition to yield a wound dressing.

### Alginate Dressings

In some embodiments, the wound dressing is an alginate dressing. For example, in certain aspects, the disclosure relates to a wound dressing for treating a diabetic ulcer wound, comprising an alginate hydrogel comprising a therapeutically effective amount of a recombinant RNA molecule encoding one or more polypeptides selected from the group consisting of chitinase-3-like protein 1 (CHI3L1), a fibroblast growth factor (FGF), interleukin-2 ORF7 (IL-2 ORF7), interleukin-2 (IL-2), an interleukin-17 (IL-17) protein, and an IL-17 receptor, wherein the recombinant RNA molecule is comprised within a lipid nanoparticle (LNP), and wherein the recombinant RNA molecule is distributed throughout the alginate hydrogel for release onto a diabetic ulcer wound during a delivery period.

In certain aspects, the disclosure relates to a. wound dressing for treating a diabetic ulcer wound, comprising an alginate hydrogel comprising a therapeutically effective amount of a recombinant RNA molecule encoding one or more polypeptides selected from the group consisting of chitinase-3-like protein 1 (CHI3L1), fibroblast growth factor 2 (FGF-2), interleukin-2 ORF7 (IL-2 ORF7). interleukin-2 (IL-2), and interleukin-17A (IL-17A), wherein the recombinant RNA molecule is comprised within a lipid nanoparticle (LNP), and wherein the recombinant RNA molecule is distributed throughout the alginate hydrogel for release onto a diabetic ulcer wound during a delivery period.

Alginate dressings are wound coverings made from a polymer derived from seaweed. Such dressings maintain a moist microenvironment that promotes healing. They can be biodegradable and have been successfully used for a variety of secreting lesions. The dressing limits accumulation of wound secretions and minimizes bacterial contamination. Alginate dressings are described, for example, in U.S. Pat. No. 10,016,524, which is incorporated by reference herein in its entirety.

Alginate is a linear polysaccharide consisting of (1,4)-linked b-D-mannuronate (M) and its C-5 epimer α-L-guluronate (G). The monomers can appear in homopolymeric blocks of consecutive G-residues (G-blocks), consecutive M-residues (M-blocks), alternating M and G-residues (MG-blocks) or randomly organized blocks. Chemical composition, primary structure and average block lengths are conveniently determined by NMR spectroscopy. Commercial alginates are generally extracted from brown algae, and the relative amount of each block type varies with the origin of the alginate. Physical-chemical and biological properties of alginate vary widely with chemical composition. G-blocks form stable cross-linked junctions with divalent cations (e.g. Ca2+, Ba2+, Sr2+, among others) leading to a three-dimensional gel network. Alginate can also form gels under acidic conditions without cross-linking agents.

Preparation of alginate dressings is described, for example, in U.S. Pat. No. 10,016,524. In some embodiments, the alginate dressing is prepared by initially molding the alginate and recombinant nucleic acid molecule (e.g., RNA molecule) into the proposed end shape of the dressing. For example, the dressing may be fabricated in two forms: alginate alone or alginate in combination with gauze. The addition of gauze to the dressing increases the mechanical stiffness and strength of the dressing and improves handling. Afterwards, the alginate alone dressing or alginate+ gauze dressing is passed through a freeze-drying process to form cryo-organized structures. One or more recombinant nucleic acid molecules (e.g., RNA molecules) is added to the alginate solution before the induction of cryo-organization (freezing) or after the lyophilization step. The terms freeze-dry and lyophilization are synonymous and used interchangeably herein. The process imparts a microstructure in the uncross-linked alginate prior to it being cross-linked. The alginate solution is chilled at the desired temperature to assure a completely frozen state. (typically at least 8 hours, e.g., overnight). Lyophilization is carried out under standard conditions, e.g., at approximately 150 millibars of pressure over a period of 1-3 days to dry the composition following freezing. For example, alginate alone (without gauze) is suitable for directly contacting a wound. In other embodiments, the alginate contains a woven mesh (e.g., gauze) interspersed within the layer of alginate. In this case, in order to fix the alginate portion of the bandage to a wound, the alginate layer (with or without a woven mesh) is placed on the wound site (where the alginate is shaped to the size of the wound or greater). A standard wound cover (e.g., TEGADERM™) is overlaid on the alginate bandage, extending in all directions past the wound margin and attaching to the skin.

In some embodiments, a wound dressing is constructed by contacting an alginate sheet with a backing material (e.g., woven or non-woven backing material, e.g., plastic, latex, gauze, cloth, or film). Exemplary backing materials include MEPITEL™, JELONET™ OPSITE™, TEGADERM™, CARBOFLEX™, LYOFOAM C™, silicone (e.g., silicone tape), or cotton). The backing material can be porous or non-porous. For example, the backing material comprises a tape or adhesive, e.g., an adhesive plastic strip or an adhesive medical tape. For example, the contacting step is performed by gluing, fusing, spraying (e.g., spraying foam onto), or otherwise attaching the alginate sheet with the backing material. In some examples, a wound dressing comprises a backing material and alginate containing a woven mesh interspersed within a layer of alginate.

The presence of a backing material and/or woven mesh on or within the alginate in the bandage provides increased durability, guard against moisture loss, and protection against abrasion compared to alginate alone.

Pore size is controlled by the temperature at which the alginate solution is frozen and the rate of temperature change. Ice crystal size is dependent on the rate of freezing. The solution is frozen, e.g., by placement in a constant temperature device, at −20° C. (standard freezer), −80° C. (deep freezer), or using liquid nitrogen, e.g., −160° C., −180° C., −200° C., or any temperature in between to customize pore size (average diameter). For example, pores with 300-800 μm diameter, e.g., 500 μm diameter, are formed at −20° C.; pores with 100-400 μm diameter, e.g., 100-200 μm diameter, are formed at about −70 to −88° C.; and pores with 10-99/100 μm diameter, e.g., 50 μm diameter, are formed using liquid nitrogen, e.g., at about −180° C. The pores are interconnected and generally homogeneous throughout the alginate composition. To promote formation of homogeneous pores, the mold into which the alginate solution is poured is optionally pre-chilled/frozen prior to pouring the solution into the mold. To form heterogeneous pores, e.g., pores that are oriented or form channels, cryo-organization is induced by creating a temperature gradient. For example, a temperature gradient is created by placing the alginate solution-containing mold between 2 plates, one plate having a warmer temperature than the other plate. In this manner, ice crystal formation occurs in a single direction, e.g., from the cold surface toward the warmer surface.

During the rapid cross-linking phase a minimum volume of calcium chloride solution (100 mM) is used to crosslink the device. By rapid crosslinking phase is generally meant one hour or less, e.g., 5, 10, 15, 30, 45 minutes. Typically, crosslinking time is about 15 min. The cryo-organized alginate structure is dry (freeze-dried) prior to being contacted with the ionic crosslinking solution. Thus, the volume of crosslinking solution added to the dry composition is at least the volume of the composition and typically 1-10 times, e.g., 1-2 times, the volume of the composition (mold volume).

The rapid crosslinking in a small volume ensures that the microstructure of freeze-drying is maintained and a minimum amount of drug is lost. An intermediary freeze-drying step may be used to create micropores in the alginate that introduce flexibility into the sheet.

Different strategies may be used to ionically crosslink the cryo-organized alginate. In some embodiments, a minimum volume of an aqueous calcium chloride solution (100 mM), e.g., calcium chloride dissolved in a physiologically acceptable buffer such as phosphate buffered saline (PBS) or HEPES buffer, is used to crosslink the device. The rapid crosslinking in a small volume ensures that the microstructure of freeze-drying is maintained and a minimum amount of drug is lost. A second method has been used to crosslink alginate in a non-aqueous solvent such as ethanol. An advantage of non-aqueous crosslinking solution is that it minimizes swelling of the alginate. A macroporous cryo-organized structure can be formed after freeze-drying an aqueous solution of alginate. The lyophilized cryo-organized alginate may be cross-linked in a solution of ethanol containing calcium nitrate (0.2M). Alginate being insoluble in organic solvents, especially in alcohols, the cryo-organized interconnected defined polymer structure does not get disrupted due to the lack of water, which usually leads to polymer dispersion and subsequent dissolution. Ethanol crosslinking provides a better retention and definition of the initial cryo-organized microstructure when compared to the aqueous crosslinking. Therefore, the macroporous structure is preserved during the calcium cross-linking process while entrapping molecules of drugs within the polymer walls.

Different options are available for the final step with regard to the aqueously crosslinked alginate structure. In some embodiments, the device is cross-linked and applied immediately to the wound. In this embodiment, the sterile freeze-dried alginate sheet+drugs and the sterile calcium chloride cross-linking solution may both supplied to the end user in a ‘smart packaging’ design that separates the alginate from the crosslinking solution until the user activates a push-and-pop mechanism (smartpack) that brings them together to permit easy cross-linking. This configuration is particularly applicable to patients with diabetic foot ulcers as they are familiar with such a system. Diabetic patients with skin/tissue ulcers such as foot ulcers already pre-wet bandages in saline before applying them, and this approach only differs in that the pre-wetting solution corresponds to the aqueous crosslinking solution, e.g., calcium chloride in water or aqueous buffer. The freeze-dried alginate bandage packaged dry, and the user contacts the dry bandage with the crosslinking solution immediately prior to applying the bandage to the skin or other tissue to be treated.

Alternatively, since the cross-linking process maintains the microstructure, it may be cross-linked by the manufacturer and once again freeze-dried for long-term storage. Testing data in which the bandages underwent the dual freeze-drying process indicated that that the device maintains its desirable flexibility and mechanical properties after the second freeze-drying step. See U.S. Pat. No. 10,016,524. Likewise, for the ethanol cross-linking route, freeze-dried cryo-organized alginate-based drug-loaded bandages are readily provided for long-term storage or embedded in a saline solution for immediate or short-term use. The alginate bandage may be applied to the skin using standard coverings, e.g., TEGADERM™.

In some embodiments, the alginate dressing further comprises branched polyethylenimine (bPEI), a polymer that can attract negatively charged LNP-mRNAs In some embodiments, the alginate dressing comprises 0.1%, 0.2%, 0.3%, 0.4%, 0.5%, 0.6%. 0.695%, 0.7%, 0.8%, 0.9%. 1.0%, 1.1%. 1.2%, 1.3%, 1.39%, 1.4%, 1.5%, 1.6%, 1.7%, 1.8%, 1.9%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 13.9%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 26%, 28%, 29% or 30% w/v of bPEI. In some embodiments, the alginate dressing comprises at least 0.1%, 0.2%, 0.3%, 0.4%, 0.5%, 0.6%. 0.695%, 0.7%, 0.8%, 0.9%. 1.0%, 1.1%. 1.2%, 1.3%, 1.39%, 1.4%, 1.5%, 1.6%, 1.7%, 1.8%, 1.9%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 13.9%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 26%, 28%, 29% or 30% w/v of bPEI. In some embodiments, the alginate dressing comprises less than 0.1%, 0.2%, 0.3%, 0.4%, 0.5%, 0.6%. 0.695%, 0.7%, 0.8%, 0.9%. 1.0%, 1.1%. 1.2%, 1.3%, 1.39%, 1.4%, 1.5%, 1.6%, 1.7%, 1.8%, 1.9%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 13.9%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 26%, 28%, 29% or 30% w/v of bPEI. Any of these values may be used to define a range for the concentration (w/v) of bPEI in the alginate dressing. For example in some embodiments, the alginate dressing comprises 0.1% to 30% w/v bPEI, 0.5% to 20% w/v bPEI, or 0.6% to 15% w/v bPEI. In a particular embodiment, the alginate dressing comprises 0.695% (w/v) bPEI. In a particular embodiment, the alginate dressing comprises 1.39% (w/v) bPEI. In a particular embodiment, the alginate dressing comprises 13.9% (w/v) bPEI.

In certain aspects, the disclosure relates to a method of preparing an alginate wound dressing, the method comprising providing an alginate solution comprising a recombinant nucleic acid molecule (e.g., RNA molecule), LNP or pharmaceutical composition as described herein, molding said alginate solution into a desired shape, inducing a cryo-organized structure by lyophilizing said molded alginate solution, and contacting said cryo-organized structure with a crosslinking agent to yield a wound dressing.

### Additional Wound Dressings

As an alternative (or in addition) to alginate, other polymers may be used to prepare the wound dressing. In some embodiments, the polymer used to prepare the wound dressing is a hydrophilic polymer, e.g., a polymer with high water absorption. In some embodiments, the polymer has strong mechanical stability from physical or chemical crosslinking. In some embodiments, the polymer is biocompatible. In some embodiments, the polymer is negatively charged. Suitable polymers include, but are not limited to, negatively charged polysaccharide polymers such as sodium carboxymethyl cellulose, hyaluronic acid, gellan gum, heparin, and glycosaminoglycan; and zwitterionic polypeptide polymers such as gelatin, collagen, and silk fibroin.

### VI. Methods of Identifying mRNAs Associated with Wound Healing

Single-cell RNA-sequencing (scRNASeq) analysis provides deep insight into cell function and disease pathophysiology by allowing the profiling of the transcriptomic landscape of individual cells in heterogeneous tissues. A recently completed study in our unit primarily focused on differences between diabetic foot ulceration (DFU) patients who heal their ulcers (Healers) and DFU patients who fail to heal them (non-Healers) and investigated molecular changes via scRNASeq analysis of surgically removed DFUs. See Theocharidis et al., 2022, Nature communications 13(1):181, which is incorporated by reference herein in its entirety. Our analysis showed enrichment of a unique population of fibroblasts, named Healing Enhancing (HE)-fibroblasts, overexpressing MMP1, MMP3, MMP11, HIF1A, CHI3L1, and TNFAIP6 genes in the DFU patients with healing wounds. Pathway analysis indicated the activation of multiple immune and inflammatory pathways including IL6, HIF1A and ILK signaling in HE-fibroblasts. Accordingly, comparing the transcriptome of individual cells from wounds that heal to the transcriptome of individual cells from wounds that fail to heal provides a method of identifying mRNAs associated with wound healing, and that may be useful in the treatment of wounds.

Methods using a pattern matching algorithm and data normalization have applied using the CMap approach to help reduce noise effects, results interpretation and strengthen the methods' reliability. For example, an important method has been introduced by Zhang et al. that has been referred to as a statistically significant connectivity map (ssCMap). See Zhang S D, Gant T. A simple and robust method for connecting small-molecule drugs using gene-expression signatures; BMC Bioinformatics 2008; 9(1):258, the entire contents of which being incorporated herein by reference. The approach uses a connectivity score computation with permutation tests at both treatment instance level and treatment set level that offers a statistical means to control over the possible false connections between the gene signature and reference profiles. See Musa et al. A review of connectivity map and computational approaches in pharmacogenomics; Briefings in Bioinformatics, 19(3), 2018, 506-523. In the present application, the differentially expressed genes between Healing Enriched (HE) fibroblasts and the normal fibroblasts as input gene signatures.

As noted in the Musa et al. publication, the CMap methodology uses the entire genomic information of the patients and of a treatment as described therein, but has the drawback that it has no specific reference to the biological functions altered by the disease in question. The ssCMap method introduces a ranking score using steps wherein the treatment and control instances are treated similarly. Genes that are affected by the treatment instance, that is, genes that are highly differentially expressed, can have more weight. Additionally, the up- and downregulated genes can be handled equally, where 2-fold of the up- or downregulation of a gene are used for the reference profile. The genes are ordered wherein the genes are ranked by their importance in the reference profile. Given the total of N genes, the procedure entails that first gene in the list will have a rank N if it is upregulated, or a rank −N if it is downregulated. The ssCMap provides for ranking a query gene signature either with an ordered or unordered gene list. The important gene expressed can be assigned a rank m or −m depending on whether it is up- or downregulated, where m is the number of genes in the gene signature. A connection strength can be calculated between reference profile R and gene signature s to measure a connection between reference profile and gene signature.

$$C ( R , s ) = {\sum }_{i = 1}^{m} R ( {g}_{i} ) s ( {g}_{i} ) \qquad ( 1 )$$

In this equation, gᵢ is the ith gene in the signature, s(gᵢ) is its signed rank in the signature and R(gᵢ) is this gene's signed rank in the reference profile (Equation 1). As noted by Zhang et al., a maximum correlation between reference profile and gene signature, can be achieved by matching m genes and their regulation status in the reference profile and the gene signature in the correct order as shown in Equation 2 below. For an unordered gene signature, all the genes in the list have equal weight because there is no particular ordering so that a maximum correlation strength for the unordered gene signature is calculated using Equation 3.

$${C}_{max}^{0} ( N , m ) = {\sum }_{i = 1}^{m} ( N - i + 1 ) ( m - i + 1 ) \qquad ( 2 )$$

$${C}_{max}^{u} ( N , m ) = {\sum }_{i = 1}^{m} ( N = i + 1 ) \qquad ( 3 )$$

The overall connectivity score (c) is calculated by dividing the connection strength with the maximum connection strength of a given gene signature and reference profile set forth in Equation 4. The connectivity score ranges from −1 to 1, where 1 indicates a maximum positive connection of gene signature with the reference profile, while −1 indicates a negative connection. To test the connection score, ssCMap uses a simple procedure to test the null hypothesis between the gene signature and the reference profile that is achieved by generating a random gene signature of the ordered/unordered list using random selection without replacement with equal probability of either up- or downregulation. After generating the signature, ssCMap calculates the connectivity score (c) of each signature as well as a “P-value” associated with the connectivity score denoted by P. As defined by Zhang et al, \tilde{c} can denote the connectivity score between a random gene signature and a reference profile. The same procedure can be repeated to estimate the sampling distribution of the random signatures. As note by Zhang et al., this provides a user-friendly software application for the ssCMap algorithm.

$$ConnectivityScore ( c ) = C ( R , s ) / {C}_{max} ( N , m ) \qquad ( 4 )$$

See Zhang S D, Gant T. “sscmap: An extensible JAVA application for connecting small molecule drugs using gene-expression signatures; BMC Bioinformatics 2009; 10: 236, the entire contents thereof being incorporated herein by reference.

In certain aspects, the disclosure relates to a method of identifying an mRNA associated with wound healing, the method comprising a) determining the mRNA expression profile of a first cell obtained from a wound that failed to heal; b) determining the mRNA expression profile of a second cell obtained from a wound that healed; and c) comparing the mRNA expression profile of the first cell and the second cell, thereby identifying an mRNA associated with wound healing. In some embodiments, the method further comprises identifying an mRNA whose expression is modulated, e.g., increased or decreased, in the second cell as compared to the first cell as an mRNA associated with wound healing. In some embodiments, the method further comprises determining the mRNA expression profile of a third cell obtained from a subject that does not have a wound, and comparing the mRNA expression profiles of the first cell, second cell and third cell.

In some embodiments, the mRNA expression profile is determined by single-cell RNA sequencing (scRNASeq). Assessing the differences in gene expression between individual cells through scRNASeq has the potential to identify rare populations that cannot be detected from an analysis of pooled cells. Methods of scRNASeq are known in the art, and are described, for example, in Hwang et al, 2018, Exp Mol Med 50, 1-14, doi.org/10.1038/s12276-018-0071-8, which is incorporated by reference herein in its entirety. Single cells may be isolated by flow-activated cell sorting (FACS). In this method, cells are first tagged with a fluorescent monoclonal antibody, which recognizes specific surface markers and enables sorting of distinct populations. Common steps required for the generation of scRNASeq libraries include cell lysis, reverse transcription into first-strand cDNA, second-strand synthesis, and cDNA amplification. In general, cells are lysed in a hypotonic buffer, and poly(A)+ selection is performed using poly(dT) primers to capture messenger RNAs (mRNAs). Commercially available sequencing platforms such as the Illumina platform (e.g., HiSeq4000, NextSeq500 and NovaSeq6000) may be used for the sequencing step.

In some embodiments, the cell isolated from the wound for sequencing analysis is selected from the group consisting of a fibroblast, keratinocyte, endothelial cell, macrophage, monocyte, and T-cell. In some embodiments, the cell isolated from the wound for sequencing analysis is a fibroblast.

In some embodiments, the first cell is obtained from a wound in a Diabetes Mellitus patient who fails to heal their diabetic wounds (e.g., diabetic foot ulcerations) (i.e., a non-Healer). In some embodiments, the second cell is obtained from a wound in a Diabetes Mellitus patient who heals their diabetic wounds (e.g., diabetic foot ulcerations) (i.e., a Healer).

In some embodiments, the wound is a diabetic wound. In some embodiments, the wound is an ulcerative wound. In some embodiments, the wound is a chronic wound. In some embodiments, the wound is a diabetic foot ulceration. In some embodiments, the wound is an acute wound.

Description of Sequences

| SEQ ID | |
|---|---|
| NO: | Description |
| 1 | Human wildtype chitinase-3-like protein 1 (CHI3L1) nucleic acid sequence |
| 2 | Human wildtype chitinase-3-like protein 1 (CHI3L1) amino acid sequence |
| 3 | Human wildtype fibroblast growth factor 2 (FGF-2) nucleic acid sequence |
| 4 | Human wildtype fibroblast growth factor 2 (FGF-2) amino acid sequence |
| 5 | Human wildtype interleukin-2 ORF7 (IL-2 ORF7) nucleic acid sequence |
| 6 | Human wildtype interleukin-2 ORF7 (IL-2 ORF7 ) amino acid sequence |
| 7 | Human wildtype interleukin-17A (IL-17A) nucleic acid sequence |
| 8 | Human wildtype interleukin-17A (IL-17A) amino acid sequence |
| 9 | Mouse wildtype interleukin-17A (IL-17A) nucleic acid sequence |
| 10 | Mouse wildtype interleukin-17A (IL-17A) amino acid sequence |
| 11 | Human wildtype interleukin-2 nucleic acid sequence |
| 12 | Human wildtype interleukin-2 amino acid sequence |
| 13 | Mouse wildtype interleukin-2 nucleic acid sequence |
| 14 | Mouse wildtype interleukin-2 amino acid sequence |

Sequences of the Disclosure
CHI3L1 nucleic acid sequence
SEQ ID NO: 1
ATGGGTGTGAAGGCGTCTCAAACAGGCTTTGTGGTCCTGG
TGCTGCTCCAGTGCTGCTCTGCATACAAACTGGTCTGCTA
CTACACCAGCTGGTCCCAGTACCGGGAAGGCGATGGGAGC
TGCTTCCCAGATGCCCTTGACCGCTTCCTCTGTACCCACA
TCATCTACAGCTTTGCCAATATAAGCAACGATCACATCGA
CACCTGGGAGTGGAATGATGTGACGCTCTACGGCATGCTC
AACACACTCAAGAACAGGAACCCCAACCTGAAGACTCTCT
TGTCTGTCGGAGGATGGAACTTTGGGTCTCAAAGATTTTC
CAAGATAGCCTCCAACACCCAGAGTCGCCGGACTTTCATC
AAGTCAGTACCGCCATTTCTGCGCACCCATGGCTTTGATG
GGCTGGACCTTGCCTGGCTCTACCCTGGACGGAGAGACAA
ACAGCATTTTACCACCCTAATCAAGGAAATGAAGGCCGAA
TTTATAAAGGAAGCCCAGCCAGGGAAAAAGCAGCTCCTGC
TCAGCGCAGCACTGTCTGCGGGGAAGGTCACCATTGACAG
CAGCTATGACATTGCCAAGATATCCCAACACCTGGATTTC
ATTAGCATCATGACCTACGATTTTCATGGAGCCTGGCGTG
GGACCACAGGCCATCACAGTCCCCTGTTCCGAGGTCAGGA
GGATGCAAGTCCTGACAGATTCAGCAACACTGACTATGCT
GTGGGGTACATGTTGAGGCTGGGGGCTCCTGCCAGTAAGC
TGGTGATGGGCATCCCCACCTTCGGGAGGAGCTTCACTCT
GGCTTCTTCTGAGACTGGTGTTGGAGCCCCAATCTCAGGA
CCGGGAATTCCAGGCCGGTTCACCAAGGAGGCAGGGACCC
TTGCCTACTATGAGATCTGTGACTTCCTCCGCGGAGCCAC
AGTCCATAGAATCCTCGGCCAGCAGGTCCCCTATGCCACC
AAGGGCAACCAGTGGGTAGGATACGACGACCAGGAAAGCG
TCAAAAGCAAGGTGCAGTACCTGAAGGACAGGCAGCTGGC
GGGCGCCATGGTATGGGCCCTGGACCTGGATGACTTCCAG
GGCTCCTTCTGTGGCCAGGATCTGCGCTTCCCTCTCACCA
ATGCCATCAAGGATGCACTCGCTGCAACGTAG
CHI3L1 amino acid sequence
SEQ ID NO: 2
MGVKASQTGFVVLVLLQCCSAYKLVCYYTSWSQYREGDGS
CFPDALDRFLCTHIIYSFANISNDHIDTWEWNDVTLYGML
NTLKNRNPNLKTLLSVGGWNFGSQRFSKIASNTQSRRIFI
KSVPPFLRTHGFDGLDLAWLYPGRRDKQHFTTLIKEMKAE
FIKEAQPGKKOLLLSAALSAGKVTIDSSYDIAKISQHLDF
ISIMTYDFHGAWRGTTGHHSPLFRGQEDASPDRFSNTDYA
VGYMLRLGAPASKLVMGIPTFGRSFTLASSETGVGAPISG
PGIPGRFTKEAGTLAYYEICDFLRGATVHRILGQQVPYAT
KGNQWVGYDDQESVKSKVQYLKDROLAGAMVWALDLDDFQ
GSFCGQDLRFPLTNAIKDALAAT
FGF-2 nucleic acid sequence
SEQ ID NO: 3
ATGGCAGCCGGGAGCATCACCACGCTGCCCGCCTTGCCCG
AGGATGGCGGCAGCGGCGCCTTCCCGCCCGGCCACTTCAA
GGACCCCAAGCGGCTGTACTGCAAAAACGGGGGCTTCTTC
CTGCGCATCCACCCCGACGGCCGAGTTGACGGGGTCCGGG
AGAAGAGCGACCCTCACATCAAGCTACAACTTCAAGCAGA
AGAGAGAGGAGTTGTGTCTATCAAAGGAGTGTGTGCTAAC
CGTTACCTGGCTATGAAGGAAGATGGAAGATTACTGGCTT
CTAAATGTGTTACGGATGAGTGTTTCTTTTTTGAACGATT
GGAATCTAATAACTACAATACTTACCGGTCAAGGAAATAC
ACCAGTTGGTATGTGGCACTGAAACGAACTGGGCAGTATA
AACTTGGATCCAAAACAGGACCTGGGCAGAAAGCTATACT
TTTTCTTCCAATGTCTGCTAAGAGCTGA
FGF-2 amino acid sequence
SEQ ID NO: 4
MAAGSITTLPALPEDGGSGAFPPGHFKDPKRLYCKNGGFF
LRIHPDGRVDGVREKSDPHIKLQLQAEERGVVSIKGVCAN
RYLAMKEDGRLLASKCVTDECFFFERLESNNYNTYRSRKY
TSWYVALKRTGQYKLGSKTGPGQKAILFLPMSAKS
IL-2 ORF7 nucleic acid sequence
SEQ ID NO: 5
ATGCAATTTATACTGTTAATTCTGGAAAAATATTATGGGG
GTGTCAAAATGTTTTACATATTACACATATTTTCAAAGAC
TTTACCTGTCTGA
IL-2 ORF7 amino acid sequence
SEQ ID NO: 6
MQFILLILEKYYGGVKMFYILHIFSKTLPV
IL-17A nucleic acid sequence (Homo sapiens)
SEQ ID NO: 7
ATGACTCCTGGGAAGACCTCATTGGTGTCACTGCTACTGC
TGCTGAGCCTGGAGGCCATAGTGAAGGCAGGAATCACAAT
CCCACGAAATCCAGGATGCCCAAATTCTGAGGACAAGAAC
TTCCCCCGGACTGTGATGGTCAACCTGAACATCCATAACC
GGAATACCAATACCAATCCCAAAAGGTCCTCAGATTACTA
CAACCGATCCACCTCACCTTGGAATCTCCACCGCAATGAG
GACCCTGAGAGATATCCCTCTGTGATCTGGGAGGCAAAGT
GCCGCCACTTGGGCTGCATCAACGCTGATGGGAACGTGGA
CTACCACATGAACTCTGTCCCCATCCAGCAAGAGATCCTG
GTCCTGCGCAGGGAGCCTCCACACTGCCCCAACTCCTTCC
GGCTGGAGAAGATACTGGTGTCCGTGGGCTGCACCTGTGT
CACCCCGATTGTCCACCATGTGGCCTAA
IL-17A amino acid sequence (Homo sapiens)
SEQ ID NO: 8
MTPGKTSLVSLLLLLSLEAIVKAGITIPRNPGCPNSEDKN
FPRTVMVNLNIHNRNTNTNPKRSSDYYNRSTSPWNLHRNE
DPERYPSVIWEAKCRHLGCINADGNVDYHMNSVPIQQEIL
VLRREPPHCPNSFRLEKILVSVGCTCVTPIVHHVA*
IL-17A nucleic acid sequence (Mus musculus)
SEQ ID NO: 9
ATGAGTCCAGGGAGAGCTTCATCTGTGTCTCTGATGCTGT
TGCTGCTGCTGAGCCTGGCGGCTACAGTGAAGGCAGCAGC
GATCATCCCTCAAAGCTCAGCGTGTCCAAACACTGAGGCC
AAGGACTTCCTCCAGAATGTGAAGGTCAACCTCAAAGTCT
TTAACTCCCTTGGCGCAAAAGTGAGCTCCAGAAGGCCCTC
AGACTACCTCAACCGTTCCACGTCACCCTGGACTCTCCAC
CGCAATGAAGACCCTGATAGATATCCCTCTGTGATCTGGG
AAGCTCAGTGCCGCCACCAGCGCTGTGTCAATGCGGAGGG
AAAGCTGGACCACCACATGAATTCTGTTCTCATCCAGCAA
GAGATCCTGGTCCTGAAGAGGGAGCCTGAGAGCTGCCCCT
TCACTTTCAGGGTCGAGAAGATGCTGGTGGGTGTGGGCTG
CACCTGCGTGGCCTCGATTGTCCGCCAGGCAGCCTAA
IL-17A amino acid sequence (Mus musculus)
SEQ ID NO: 10
MSPGRASSVSLMLLLLLSLAATVKAAAI IPQSSACPNTE
AKDFLQNVKVNLKVENSLGAKVSSRRPSDYLNRSTSPWTL
HRNEDPDRYP SVIWEAQCRHORCVNAEGKLDHHMNSVLI
QQEILVLKREPESCPFTERVEKMLVGVGCTCVASIVRQAA
IL-2 nucleic acid sequence (Homo sapiens)
SEQ ID NO: 11
atgtacaggatgcaactcctgtcttgcattgcactaagtc
ttgcacttgtcacaaacagtgcacctacttcaagttctac
aaagaaaacacagctacaactggagcatttactgctggat
ttacagatgattttgaatggaattaataattacaagaatc
ccaaactcaccaggatgctcacatttaagttttacatgcc
caagaaggccacagaactgaaacatcttcagtgtctagaa
gaagaactcaaacctctggaggaagtgctaaatttagctc
aaagcaaaaactttcacttaagacccagggacttaatcag
caatatcaacgtaatagttctggaactaaagggatctgaa
acaacattcatgtgtgaatatgctgatgagacagcaacca
ttgtagaatttctgaacagatggattaccttttgtcaaag
catcatctcaacactaacttga
IL-2 amino acid sequence (Homo sapiens)
SEQ ID NO: 12
MYRMQLLSCIALSLALVINSAPTSSSTKKTQLQLEHLLLD
LQMILNGINNYKNPKLTRMLTFKFYMPKKATELKHLQCLE
EELKPLEEVLNLAQSKNFHLRPRDLISNINVIVLELKGSE
TTFMCEYADETATIVEFLNRWITFCQSIISTLT
IL-2 nucleic acid sequence (Mus musculus)
SEQ ID NO: 13
ATGTACAGCATGCAGCTCGCATCCTGTGTCACATTGACAC
TTGTGCTCCTTGTCAACAGCGCACCCACTTCAAGCTCCAC
TTCAAGCTCTACAGCGGAAGCACAGCAGCAGCAGCAGCAG
CAGCAGCAGCAGCAGCAGCACCTGGAGCAGCTGTTGATGG
ACCTACAGGAGCTCCTGAGCAGGATGGAGAATTACAGGAA
CCTGAAACTCCCCAGGATGCTCACCTTCAAATTTTACTTG
CCCAAGCAGGCCACAGAATTGAAAGATCTTCAGTGCCTAG
AAGATGAACTTGGACCTCTGCGGCATGTTCTGGATTTGAC
TCAAAGCAAAAGCTTTCAATTGGAAGATGCTGAGAATTTC
ATCAGCAATATCAGAGTAACTGTTGTAAAACTAAAGGGCT
CTGACAACACATTTGAGTGCCAATTCGATGATGAGTCAGC
AACTGTGGTGGACTTTCTGAGGAGATGGATAGCCTTCTGT
CAAAGCATCATCTCAACAAGCCCTCAATAA
IL-2 amino acid sequence (Mus musculus)
SEQ ID NO: 14
MYSMQLASCVTLTLVLLVNSAPTSSSTSSSTAEAQQQQQQ
QQQQQQHLEQLLMDLQELLSRMENYRNLKLPRMLTFKFYL
PKQATELKDLQCLEDELGPLRHVLDLTQSKSFQLEDAENF
ISNIRVTVVKLKGSDNTFECQFDDESATVVDFLRRWIAFC
QSIISTSPQ

### EXAMPLES

This invention is further illustrated by the following examples which should not be construed as limiting. The contents of all references, Uniprot, Gene ID, Entrez Gene ID, GenBank Accession and Gene numbers, and published patents and patent applications cited throughout the application are hereby incorporated by reference in their entirety. Those skilled in the art will recognize that the invention may be practiced with variations on the disclosed structures, materials, compositions and methods, and such variations are regarded as within the ambit of the invention.

### Example 1. Healing Enriching (HE)-Fibroblasts Drive DFU Healing by Promoting Matrix Remodeling and Inflammatory Response

We investigated the molecular changes via scRNASeq analysis of skin specimens taken from surgical excision of diabetic foot ulcerations (DFUs) from patients who healed their ulcer (Healers) or failed to heal it (non-Healers). As control groups, we also performed scRNASeq analysis of intact foot skin specimens from Diabetes Mellitus (DM) patients with no DFU and healthy non-DM patients. Only focusing on cells of fibroblast identity, we found 14 distinct clusters. The evaluation of cellular makeup of clusters unveiled a higher proportion of cells (58%-90%) from DFU-Healers in specific sub-clusters; clusters 3, 4, 6 and 13. These four sub-clusters represent four heterogeneous states or subtypes of the HE-Fibroblasts (FIG. 2) that overexpress MMP1, MMP3, MMP11, HIF1A, CHI3L1, and TNFAIP6. Pathway analysis indicated that in fibroblasts from DFU-Healers there was activation of multiple immune and inflammatory pathways including IL6, HIF1A and ILK signaling and upstream regulator genes like TNF, IL6 and HIF1A.

### Example 2. Isolated Cultured Fibroblasts from Healing and Non-Healing DFUs Display Differences in Gene Expression

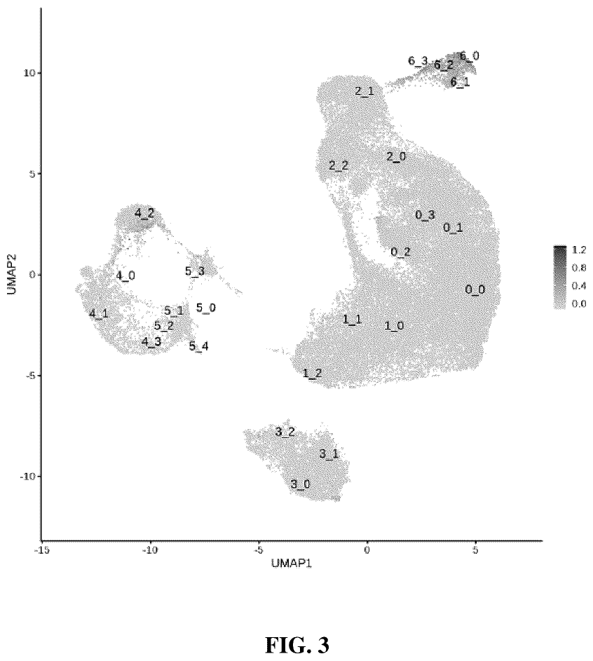

We isolated fibroblasts taken from surgical excision of DFUs of three DFU-Healers and three DFU-non-Healers and after multiple passages of in vitro culture, they were cultured with a 3D organotypic tissue scaffold. Previous studies have shown that DFU-derived primary fibroblasts generate 3D tissue-based wound healing models that mimic DFU wound repair that occurs in vivo (Kashpur et al., 2018, FASEB journal, doi: 10.1096/fj.201801059; Maione et al., 2015, Tissue engineering Part C, Methods 21(5):499-508, doi: 10.1089/ten.TEC.2014.0414). Disaggregation of the scaffold and scRNA-seq analysis revealed fibroblast heterogeneity and identified clusters 6_0, 6_1, 6_2, 6_3 that were overrepresented in the samples from DFU-Healers (FIG. 3). Cells of these clusters exhibit strong proliferating activity and trajectory analysis suggests that they may be progenitors of other fibroblast subsets. Enriched genes in the DFU-Healers included GOS2, which is implicated in cell cycle control; KAZN, implicated in cell adhesion, cytoskeletal organization, and epidermal differentiation; ACKR3, critical regulator of platelet activation, thrombus formation; COL7A1, required for re-epithelialization and support for dermal fibroblast migration; ATF5, a novel component of the HIF1 transcription complex and SOX4, which is involved in the regulation of embryonic development. Enriched genes in the DFU-non-Healers included SHOX, associated with short stature, Turner syndrome and development; CRLF1, involved in inflammatory response, ADH1B that metabolizes a wide variety of substrates, including ethanol and is known to be present in human fibroblasts (Petersen et al., 1980, Advances in experimental medicine and biology 132:533-41. doi: 10.1007/978-1-4757-1419-7_55) and PTX3, involved in regulating inflammation and complement activation. These findings indicate that 3D skin models retain cellular gene expression signatures and could facilitate clinically relevant functional investigations in vitro.

### Example 3. Evaluation of Fibroblasts Expressing CHI3L1 for Wound Healing in a Diabetic Mouse Model

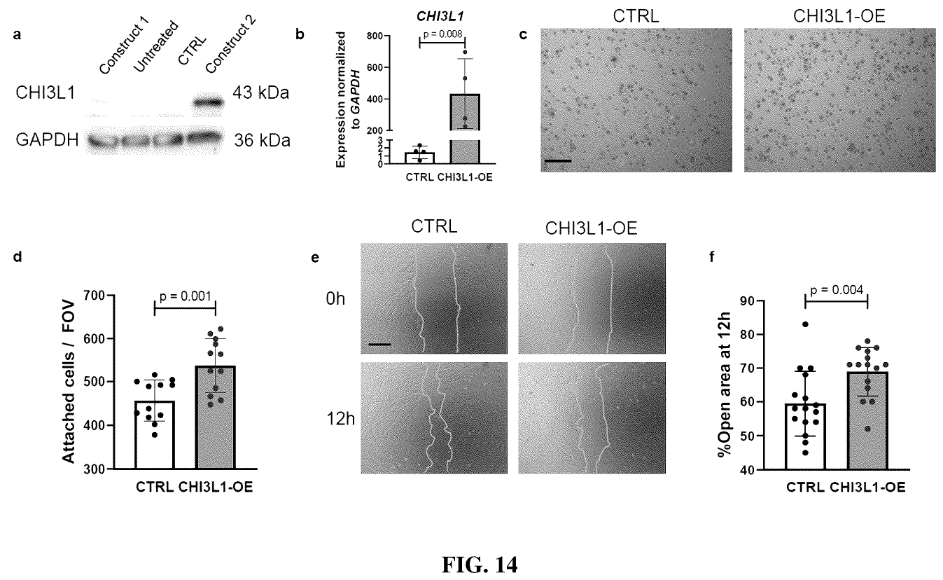

To explore the effects of healing associated fibroblasts (HE-Fibro) 1 marker genes expression in vitro, we selected one of the top enriched genes, CHI3L1, a chitinase that has been previously implicated in cancer metastasis, angiogenesis and proliferation (Libreros et al., 2013, Immunol Res 57, 99-105, doi:10.1007/s12026-013-8459-y; Luo, D. et al. 2017, Cancer Biomark 18, 273-284, doi:10.3233/CBM-160255; Morera, E. et al., 2019, In Vitro Cell Dev Biol Anim 55, 838-853, doi:10.1007/s11626-019-00403-x), and generated dermal fibroblast (Fb) cell lines transduced with lentiviral vectors overexpressing CHI3L1 (CHI3L1-OE) or a control sequence (CTRL). Western blotting demonstrated a complete lack of expression in untreated cells and RT-qPCR analyses confirmed a significant upregulation of CHI3L1 with construct 2 (FIG. 14A, 14B), which we selected for all further experiments. In adhesion assays, more CHI3L1-OE cells attached to fibronectin-coated surfaces compared to CTRL (FIG. 14C, 14D), while diminished migration was observed in scratch wound experiments (FIG. 14E, 14F). Altogether, these results shed light on the potential functional roles of the HE-Fibro, indicating that they possess enhanced adherent and decreased migratory capacities and suggest that they are firmly anchored on the ECM and mediate healing through secretion of molecules.

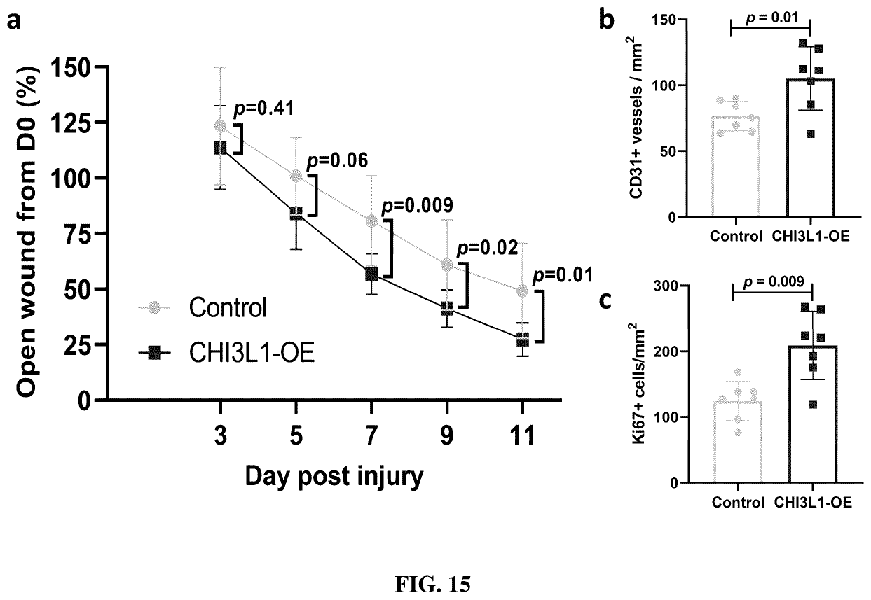

To examine the effect of CHI3L1-OE Fb in diabetic wound healing, one million control or CHI3L1-OE freshly trypsinized Fb (viability ˜99%) from passage 8 were diluted in 200 μl of serum free DMEM and kept on ice until treatment. We used an established pre-clinical model of impaired diabetic wound healing, the db/db mice. Leptin receptor -deficient (db/db) mice have an autosomal recessive mutation in the leptin receptor and present features typical of type 2 diabetes mellitus, including hyperphagia, obesity, neuropathy, hyperglycemia and severely impaired wound healing. A 6 mm biopsy punch was used to inflict two wounds on the dorsum of each mouse. The wounds were treated with four equidistant 40 l subcutaneous injections at the wound periphery, and 40 l that were dropped on the wound bed immediately after creation of 6 mm diameter bunch biopsy wounds. The wounds were then covered with an occlusive dressing (Tegaderm™) for protection. We monitored wound closure from day 3 and every two days until the conclusion of the study on Day 11 and we observed that CHI3L1-OE Fb treated wounds healed faster, with a trend towards statistical significance on Day 5 (p=0.06) and significant for all subsequent time-points, with almost twice as much healing on Day 11 (49.2±21% of open wound compared to Day 0 for control treated Fb vs 27.3±7.3% for CHI3L1-OE Fb) (FIG. 15A). In addition, there were more CD31+ blood vessels in the granulation tissue of CHI3L1-OE Fb treated wounds, indicating elevated angiogenesis (FIG. 15B), as well as increased numbers of Ki67+ dermal cells demonstrating enhanced proliferation (FIG. 15C). Overall, these findings suggest that application of CHI3L1-OE Fb on diabetic wounds leads to accelerated wound healing by affecting multiple pivotal processes on different time points.

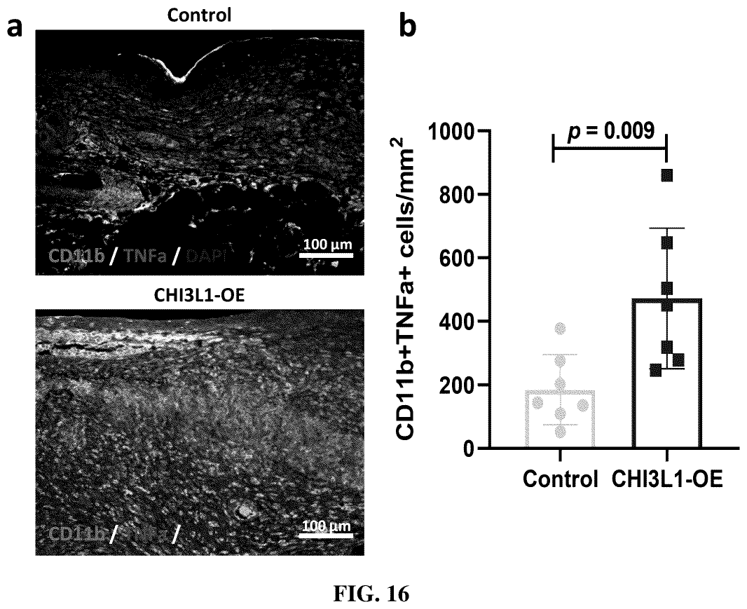

Furthermore, immunofluorescence staining of Day 11 wounds treated with either CHI3L1-OE or control Fb using pan-macrophage (Mf) marker CD11b and M1 associated marker TNFa, revealed a substantially higher density of CD11b/TNFα double positive cells in the CHI3L1-OE Fb treated wounds, by almost 2-fold (FIG. 16A, 16B). This further highlights the importance of mounting an acute-like inflammatory response for successful healing of diabetic wounds and suggests that CHI3L1-OE Fb and M1 Mf work together towards this and is also in accordance with healing human DFUs containing higher numbers of M1 associated Mf.

### Example 4. LNP-mRNA Cellular Uptake

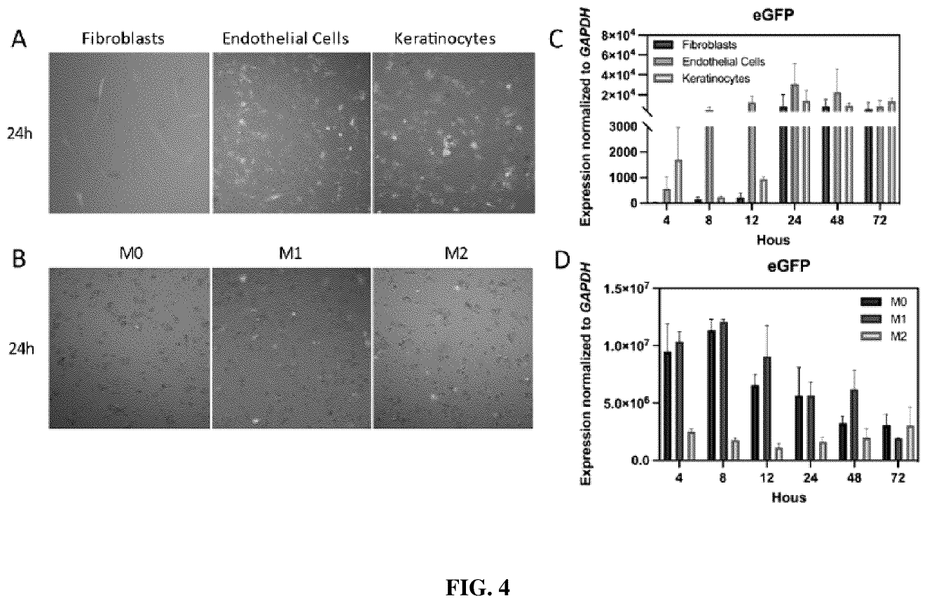

To determine LNP-mRNA cellular uptake and delivered gene expression, eGFP (enhanced green fluorescent protein) mRNA was selected as a reporter gene. LNP-eGFP mRNA was formulated through GenVoy-ILM assembler at a specific RNA working concentration and flow rate. Human fibroblasts, endothelial cells, keratinocytes, naïve macrophages (M0), LPS induced macrophages (M1), and IL-4/IL-13 induced macrophages (M2) were treated with LNP-eGFP mRNA for up to 72h. Strong eGFP expression was observed in all cell types examined, suggesting that the LNPs are readily taken up (FIG. 4). Of interest, eGFP expression was the highest during the first 24 hours in macrophages and between 24-72 hours in the remaining cell types.

### Example 5. In Vitro Migration of Endothelial Cells, Keratinocytes, and Human Dermal Fibroblasts Treated with LNP-mRNA

### Methods

Endothelial cells, human dermal fibroblasts, and keratinocytes were plated at 100K, 50K, and 100Kcells per well in 24-well plates, respectively. The cell monolayer was scratched in a straight line using a 200 μl pipette tip. Debris were removed by washing once with PBS. Cells were incubated in 500 ng/ml LNP-mRNA throughout the experiment. Two mRNAs were tested, CHI3L1 and IL-2 ORF7. An empty LNP was also tested as a negative control. Images were taken immediately after the scratch, in 6 h, 12h, and 24h for four wells per condition. Scratch areas were analyzed using ImageJ/FIJ. Three independent experiments were performed.

### Results

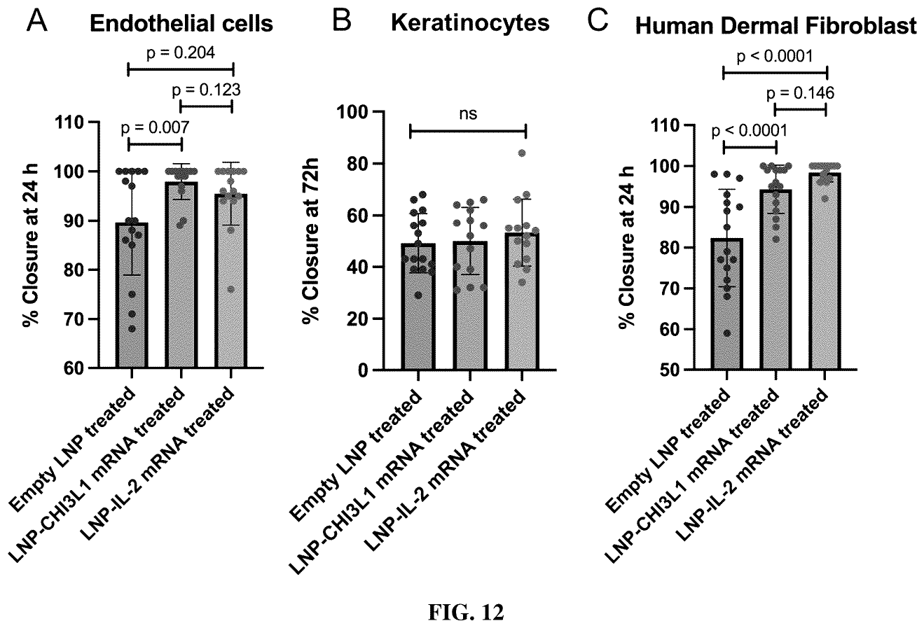

As shown in FIG. 12A, treatment of endothelial cells with CHI3L1 mRNA or IL-2 ORF7 mRNA increased endothelial cell migration relative to the empty LNP negative control. As shown in FIG. 12C, treatment of human dermal fibroblasts with CHI3L1 mRNA or IL-2 ORF7 mRNA also increased cell migration. Treatment of keratinoctyes with CHI3L1 mRNA or IL-2 ORF7 mRNA did not affect cell migration. See FIG. 12B.

### Example 6. Development and Characterization of Modified-mRNA-Releasing Alginate Bandages

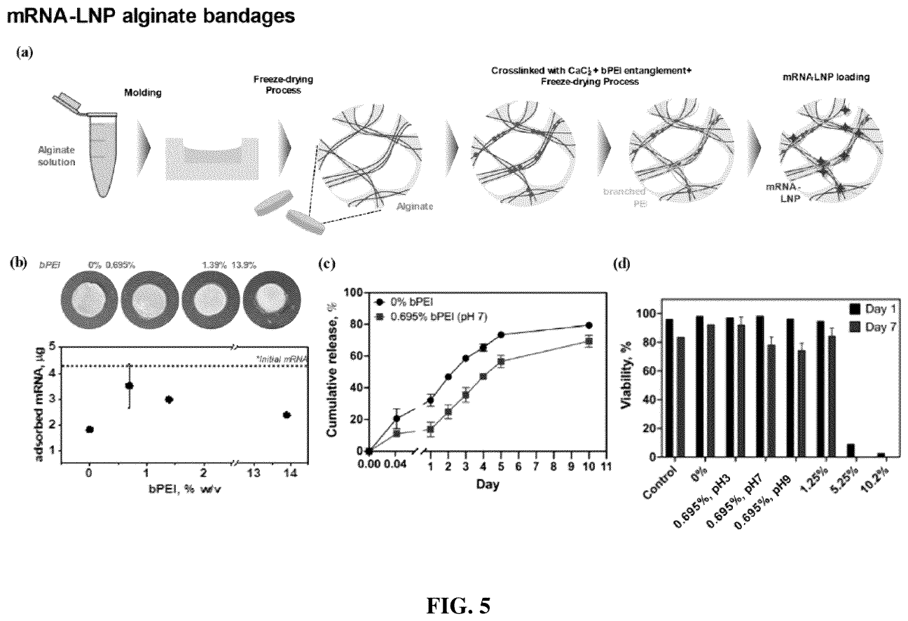

Alginate (Alg) solutions were placed into pre-formed molds, frozen, and lyophilized to micron-sized pores in the bandages (FIG. 5). The hydrogels were then cross-linked with calcium chloride, and entangled with positively charged branched Polyethylenimine (PEI) a polymer which can attract the negatively charged LNP-mRNAs, and frozen, re-lyophilized to form positively charged, dry, off-the-shelf bandages that can be stored and applied LNP-mRNAs before placed on wounds. The LNP-mRNAs loaded hydrogel (initial load: 4 μg) submerged in Hank's balanced salt solution (HBSS). Release study: The supernatant from low (0.695%), medium (1.39%), and high (13.9%) bPEI concentration modified hydrogels at earlier time point were collected. The RiboGreen assay determined the release of LNP-mRNA from bPEI-Alg gels.

### Example 7. Application of LNP-mRNA Loaded Bandages to Diabetic Mouse Dorsal Wounds Enhances Wound Healing

### Methods

Development of LNP-mRNAs. To deliver mRNAs in the most efficient way, one open reading frame from each gene of interest (CHI3L1, IL-2 ORF7, FGF-2, and IL-17A) was first determined from their gene sequence obtained from the NCBI database. We specifically selected an open reading frame that is present in both the mouse and the human gene to maximize translatability for the diabetic mouse model, humanized mouse model, and potential human clinical trial in the future. For IL-17A, the mouse version of the gene was used. Comparably, a specific open reading frame from every gene of interest will be determined again when we employ the porcine model. Following the selection of the open reading frame from each gene of interest, we finalized their modification with full substitution of both Pseudo-U and 5-Methyl-C to reduce immunogenicity. All modified mRNAs were purchased from Trilink BioTechnology and lipid nanoparticles from Percision NanoSystems. Each mRNA contained a 5′ cap structure, a 5′ UTR, a 3′ UTR and a polyA tail. The mRNAs were not codon optimized. Each individual LNP-mRNA was formulated through GenVoy-Ionizable Lipid Mix (ILM) assembler at a flow rate of 12 ml/min. The working concentration of mRNA was prepared at 0.15 mg/ml for the best LNP encapsulation result. RiboGreen assay was performed to determine mRNA concertation after their formulation. The mRNA encapsulation efficiency we routinely obtain is greater than 93%. All newly formulated LNP-mRNAs were adjusted to working concentration (100 μg/ml) and 40 ul of LNP-mRNA solution was loaded onto each macroporous alginate hydrogel. The release rate of modified-mRNA-LNPs is controlled by the chemistry of the novel hydrogel bandage.

Development of bandages that can provide controllable release of one or multiple mRNAs in the wound area. We developed bandages from alginate and branched poly (ethylene imine) to provide a sustained (>7 days) release of mRNA-LNP after placement on wounds. The 2 w/v % alginate solutions placed into pre-formed teflon molds, frozen, and lyophilized to have entangled alginate network with micron-sized pores in the bandages. The cryogels were then cross-linked with 100 mM calcium chloride, and entangled with bPEI, and frozen, re-lyophilized to form positively charged, dry, off-the-shelf bandages that can be stored and applied mRNA-LNP before placed on wounds. The electrostatic interaction between bPEI-Alginate bandages and mRNA-LNP offers continuous and sustained release. To optimize the composition of bandages, we performed mRNA-LNP release study and 3T3 cell viability study by varying the concentration or pH of entangled bPEI. The relative amount of bPEI on bandages was determined by FT-IR. For the release study, we collected the media at every time point and analyzed the release of mRNA-LNP from bPEI-Alginate bandages by using the RiboGreen assay. For viability study, 3T3 cells were incubated with bPEI-Alginate bandages and analyzed for viability at days 1 and 7.

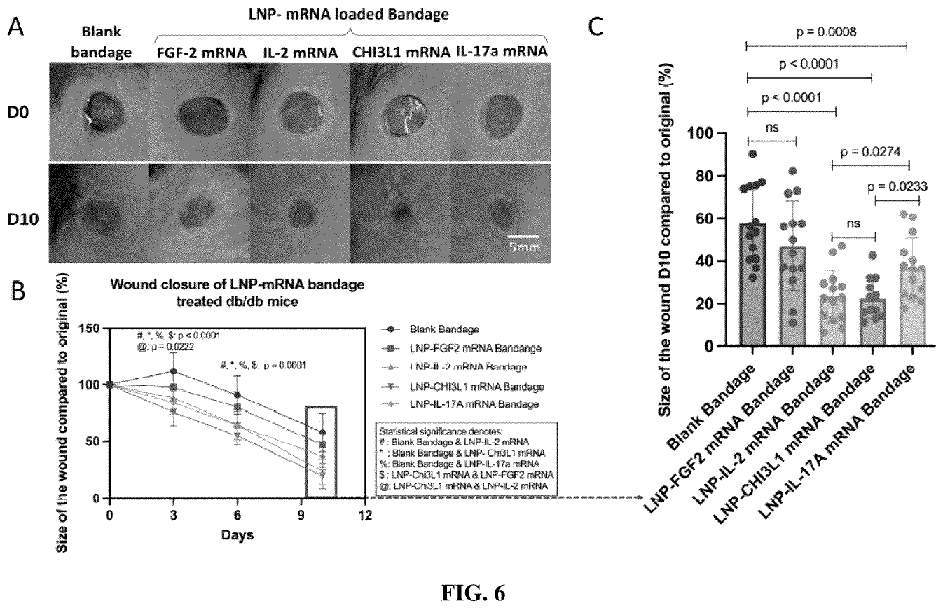

Two 6-mm punch biopsy full-thickness wounds were created on the dorsum of 16-week-old diabetic db/db mice (FIG. 6A). Macroporous bandages loaded with 4 μg of LNP-FGF2, LNP-IL-2 ORF7, LNP-CHI3L1, or LNP-IL-17A mRNA were topically applied immediately on the wounds for 10 days. The wound size was measured every three days.

### Results

Repeated measurement analysis using the general linear model showed that all four treatments performed better than the blank bandage during the whole period of the experiment (FIG. 6B). IL-17A and IL-2 ORF7 performed better than FGF-2 while CHI3L1 performed better than all other treatments. On Day 10, wound reduction was similar between blank and FGF-2, while wound size was lower in IL-17A and similarly the lowest in CHI3L1 and IL-2 ORF7 treatments (FIG. 6C). These results indicate that delivery of mRNAs encoding CHI3L1, IL-2 ORF7, FGF-2, or IL-17A to diabetic wounds is effective in enhancing wound healing.

### Evaluation of Combined Treatments

As combining treatment with various mRNAs can be easily achieved, we compared the effect of combined CHI3L1 and IL-2 ORF7 treatment for ten days versus treatment with IL-2 ORF7 or CHI3L1 alone, using the same animal model and techniques as in the previous experiments. Although the combined treatment did not perform better at Days 3 and 6 post-wounding, it tended to be lower at Day 10. For example, the size of the wound compared to the original (%) was: CHI3L1: 22±10, IL-2 ORF7: 23±12, IL2 & CHI3L1 17±13 (P═NS). The lack of significance was probably related to the fact that the wound reduction achieved on Day 10 is high, close to almost complete healing.

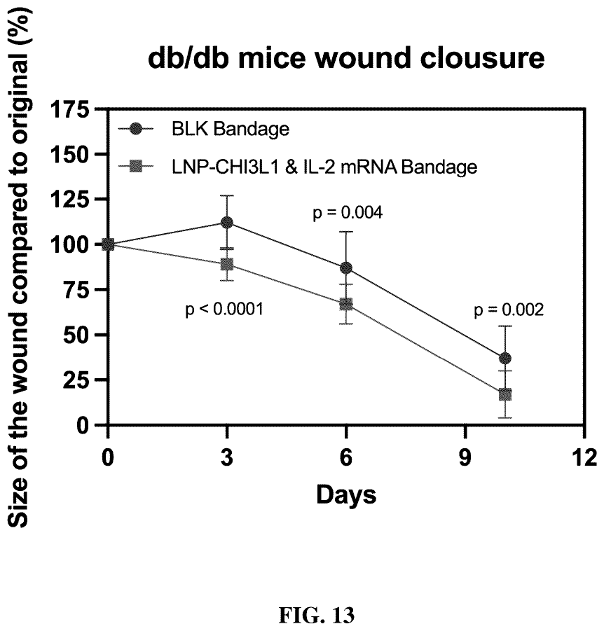

The combination of CHI3L1 and IL-2 ORF7 mRNAs was evaluated in a further experiment. Two circular biopsy punch 6-mm (Integra Miltex) full-thickness wounds were created on 16-week old male db/db mice dorsum, as in the experiments above. A microporous bandage was loaded with 2 μg of LNP-Chi3L1 mRNA and 2 ug of LNP-IL-2 ORF7 mRNA topically applied immediately onto a 6-mm wound for 10 days. Wound size was measured every 3 days after bandage application. As shown in FIG. 13, application of an LNP-Chi3L1 and IL-2 ORF7 mRNA loaded bandage to diabetic mouse dorsal wound enhanced wound healing relative to a bandage that did not contain mRNA. These results indicate that combination of treatments may be better than single mRNA treatment and may be further tested in either larger wounds and/or other animal models, such as the diabetic porcine model.

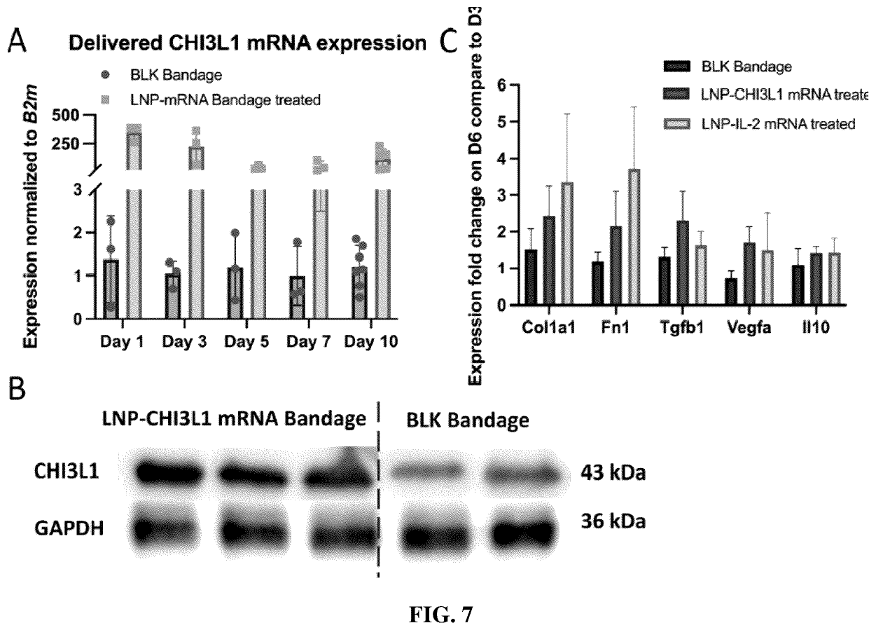

Application of LNP-CHI3L1-mRNA bandages resulted in increased delivered mRNA and protein expression in the treated wounds and enhanced the expression of healing associated markers. For example, we evaluated the presence of delivered CHI3L1 mRNA in wounds treated with LNP-CHI3L1 mRNA or blank bandage in diabetic mice that were sacrificed on Days 1, 3, 5, and 10. CHI3L1 was present at high levels during all these time points (FIG. 7A). In addition, there was more than two-fold CHI1L3 protein expression at Day 10 (FIG. 7B). In another experiment, treatment with CHI3L1 mRNA increased the mouse expression of genes related to wound healing at Day 6 when compared to Day 3 (FIG. 7C). In contrast, we did not identify any delivered IL-2 ORF7, IL-17A and FGF-2 mRNA on Day 10, suggesting that higher doses or more frequent dosing may be required to achieve maximum results with these treatments.

### Example 8. Evaluation of mRNA Efficacy in an Ex Vivo 3D Human Skin Equivalent

Delivery of modified-mRNA-LNPs therapeutics is evaluated to provide proof of their efficacy in diabetic wounds in an ex vivo 3D human skin equivalent. Alginate hydrogels fabricated into bandages are used to deliver mRNA-LNPs in ex vivo 3D human skin equivalents. In previous studies, primary fibroblast cell lines were isolated from DFU patients and healthy control patients (NFF) (Kashpur et al., 2018, FASEB journal, doi: 10.1096/fj.201801059; Maione et al., 2015, Tissue engineering Part C, Methods. 21(5):499-508, doi: 10.1089/ten.TEC.2014.0414). We have published that when these DFU-derived fibroblasts are incorporated into a 3D skin-like tissue wound healing assay they display tissue phenotypes consistent with their in vivo properties of delayed wound healing. By using these 3D skin equivalents, the most efficient mRNA combinations and the most appropriate dosing and timing of delivery that can improve diabetic wound healing are identified. As rigorous experimental design requires that we are sufficiently powered for statistical characterization, LNPs are tested in 3D tissue wound healing assays constructed with DFU patient-derived fibroblast cell lines from 7 diabetic patients with chronic ulcers (DFU) that were from healing fibroblasts (HE) and 7 fibroblast that were from non-healing (NH) DFU patients previously been characterized in our published work (Maione et al., 2016, Wound repair and regeneration: official publication of the Wound Healing Society [and] the European Tissue Repair Society, doi: 10.1111/wrr.12437. PubMed PMID: 27102877; Park et al., 2014, Epigenetics 9(10):1339-49. doi: 10.4161/15592294.2014.967584). Various combinations of four different LNP mRNAs (CHI3L1-mRNA, IL-17A-mRNA, FGF-2 and IL-2 ORF7 mRNA) are delivered directly to these DFU-HE and DFU-NH, 3D skin-like tissue wound healing assay to test our hypothesis that topical wound treatment can release mRNA with specific temporal delivery to promote DFU healing. These 3D tissues are wounded using a 3 mm punch biopsy and re-epithelialization monitored for 72 hours in the presence or absence of various combinations of four different LNP mRNAs. Following wounding of 3D skin-like tissues, epithelialization is measured after 72 hours and degree of closure from the wound margin is calculated to quantify re-epithelialization in multiple tissue sections. The degree of wound coverage by surface keratinocytes of DFU-HE-containing tissues is measured and compared to wound coverage of DFU-NH-containing tissues and expressed as the % of wound re-epithelialization. Fibroblasts from tissues showing greatest wound healing is evaluated in single cell studies similar to those performed in the examples above.

### Example 9. Evaluation of Efficacy of Bandage-Released mRNAs, Either Alone or in Combination, in Improving Wound Healing in Diabetic db/db Mice

The efficacy of bandages releasing LNP-IL-2 ORF7 mRNA, LNP-CHI3L1-mRNA, LNP-FGF2-mRNA and LNP-IL-17A-mRNA is evaluated at different doses and times. The same animal model and the same methodology described in Example 7 above are used, e.g., 16-week-old male and female db/db mice as previously described by us (Tellechea et al., 2013, The international journal of lower extremity wounds 12(1):4-11, doi: 10.1177/1534734612474303). For single therapies, two 6 mm full-thickness skin wounds are created on their back. For the combined treatments, one larger wound 10×10 mm is created. Therapeutic effect on wound closure, including re-epithelialization and granulation tissue formation is analyzed as previously published (Tellechea et al., 2019, The Journal of investigative dermatology, doi: 10.1016/j.jid.2019.08.449). Wounds are harvested after animal sacrifice and are used in mechanistic studies.

One order of magnitude lower and higher dose from the one employed in Example 7 above are delivered for all four mRNAs to identify the optimal dose. In addition, treatment with the best performing dose for FGF-2, IL-2 ORF7 and IL-17A is repeated on Days 3 and 5 post-wounding. Finally, as previous data indicates that FGF-2 and IL-17A act during the proliferation phase, in separate experiments, treatment is initiated at Day 5, when the transition from inflammation to proliferation phase occurs. Given the fact that CHI1L3 mRNA performed consistently well and both delivered mRNA and protein expression were present at Day 10, this factor is delivered once at the initiation of treatment in proposed experiments. The completion of these experiments will allow us to estimate the optimal combinations of treatments regarding both the best factors and the best timing of delivery. We expect that up to eight such combined treatments, with two or three different mRNAs will be tested to select the best four options that will be evaluated in the porcine model.

At either day 3, 5 or 10 post-wounding, mice are euthanized and wound tissue is harvested for histological and molecular analyses. Wound healing is monitored using standardized photography daily during the course of 3, 5 and 10 days. The following analyses are also performed: 1) H&E staining to evaluate inflammatory or immunological response to the treatment (in vivo toxicity); 2) Immunohistochemical staining to assess angiogenesis (CD31 and vWF); 3) immunofluorescent staining to evaluate the M1/M2 macrophage status. 4) protein levels of factors involved in wound healing, such as VEGF, MMP-9, IL-6, KC, NEP, VEGFR1/2, PDGF, TNFα, SP, SDF1α, MMP-2 and NF-kB. Their expression is evaluated both in unwounded skin (day 0) and wounded skin (days 3, 5 and 10) by qRT-PCR and western blot/immunohistochemistry using standard techniques; 5) serum protein levels of inflammatory cytokines and biochemical markers of endothelial function are measured using a Luminex 200 apparatus (Luminex, Austin, TX).

Our preliminary data indicate no systemic effects from topical mRNA application. However, to further exclude such possibility, additional experiments are performed using bandages that deliver LNP-eGFP mRNA and expression in various tissues and organs is determined.

Metrics of success. The main endpoint is the achieved wound reduction. The secondary endpoints include re-epithelialization, granulation tissue formation and angiogenesis as previously described (Tellechea et al., 2019, The Journal of investigative dermatology, doi: 10.1016/j.jid.2019.08.449; Leal et al., 2015, The American journal of pathology 185(6):1638-48, doi: 10.1016/j.ajpath.2015.02.011; Tellechea et al., 2013, The international journal of lower extremity wounds 12(1):4-11, doi: 10.1177/1534734612474303). Additional secondary endpoints include inflammatory cell infiltration, collagen deposition, skin hemoglobin oxygen saturation and M1/M2 macrophage ratio.

Anticipated Results. We expect that mRNA treatment, especially treatment with combination of two or more factors, will greatly improve wound healing in diabetic mice.

Power analysis. The results presented above in Example 7 showed that when comparing mice wounds treated with control versus those treated with the four mRNA treatments, at Day 10, we had a pooled standard deviation of 16 and a maximum difference of 36, which indicates that 6 or 8 mice in each group will allow us to detect a difference in wound healing improvement with more with 80% or 90% power and 5% level of significance respectively. Similar results were observed for the other time points. In order to cover possible attrition due to animal loss during the whole experiment, we will therefore include 10 mice in each group to ascertain that we will capture microvariations within groups or phenotypes of toxicity at the molecular level.

Statistical analysis. Comparisons between two groups is performed using the t test for normally distributed data and the Mann-Whitney test for data that are not distributed normally. When more than two groups are compared, the ANOVA and Kruskal-Wallis tests are used respectively.

### Example 10. Investigating Efficacy in a Large Animal Model (the Diabetic Yucatan Minipig) and Confirming Mechanisms of Action in Minipig and Mouse Models

Rationale. It is currently recommended that two animal models are tested for evaluating treatment for diabetic wounds, and we have chosen to test the diabetic mouse model described above and diabetic Yucatan miniature pig model (Matoori et al., 2021, Science translational medicine 13(585), doi: 10.1126/scitranslmed.abe4839). The skin of pigs is similar to human skin in many aspects, including thickness of epidermis, collagen composition, hair follicle density, adherence to the underlying structures, rete ridges, epidermis turnover and types of immune cells (Seaton et al., 2015, ILAR J. 2015; 56(1):127-38; doi: 10.1093/ilar/ilv016). Our choice of Yucatan miniature pigs is based on the fact that they are very similar to Yorkshire pigs, which have already been shown to have diabetes-related impaired wound healing akin to the human condition, but their smaller size makes them more manageable (Velander et al., 2008, Wound repair and regeneration: official publication of the Wound Healing Society [and] the European Tissue Repair Society 16(2):288-93, doi: 10.1111/j.1524-475X.2008.00367.x). Our unit has experience in successfully employing this animal model in previous studies that have allowed us to collect a significant amount of transcriptomic and proteomic data (Theocharidis et al., 2022, Nat Biomed Eng., doi: 10.1038/s41551-022-00905-2).

In addition to the pig studies, in this experiment the mechanism of action of the mRNAs is evaluated by employing integrated bulk, single cell and spatial transcriptomic and proteomic analysis. This analysis provides valuable information about possible differences between mice, pig and human tissue.

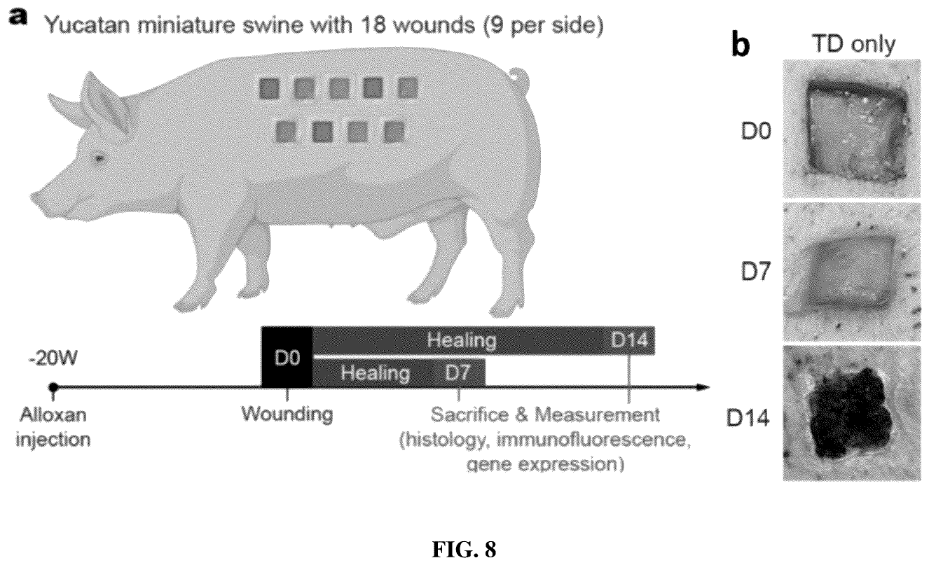

Research Design. Porcine Studies. Male and female diabetic Yucatan pigs are evaluated. Diabetes is induced using alloxan by the vendor before their arrival to our research facility as previously described (Velander et al., cited above). Two weeks after diabetes induction 24 square (15×15 mm) excisional full thickness wounds are created on the paravertebral and thoracic area using an acrylic board (20 by 30 cm) template with a distance of 2.5 cm of unwounded skin between each wound (FIG. 8) as previously described (Theocharidis et al., 2022, Nat Biomed Eng., doi: 10.1038/s41551-022-00905-2).

The best four treatments (named A, B, C, D) that are identified in the mice studies are evaluated. In addition to this, the following two conditions are also tested: E.) Blank bandage+bPEI+Laponite only and F.) Tegaderm only covered wounds. Each treatment is tested in 12 wounds, initially employing three pigs (two females, one male). Each treatment is applied on four wounds of each animal in a way that all four treatments contain wounds with similar characteristics regarding their distribution and anatomical position, including distance from the head and the spine. All wounds are covered with Tegaderm. Bandages are replaced at Day 5 and 10 post-wounding with new ones and animals are euthanized at Day 14 post-wounding. The best two performing treatments are further tested in three more pigs, (two males and one female). One pig is sacrificed at Day 5, one at Day 21 and one at Day 28. Bandages changes and wound measurements are performed every five days. Final wound size assessment and wound harvest is done on the day animals are sacrificed. Upon sacrifice, all wounds and surrounding skin is excised and the same assays that are described in the mice section are performed. This design allows a complete picture of the action of the selected treatments during all phases of wound healing.

### Example 11. Mice, Human and Porcine-Omic Studies

To further investigate the mechanisms of action of the best performing treatments, transcriptomic and proteomic studies are performed in the DFU fibroblasts from the ex vivo experiments and mice and porcine wound tissues.

Bulk RNAseq: Temporal whole transcriptome (bulk) analysis is performed with four biological replicates in each group. Wounds from all previously described experiments involving both mice and pigs, are analyzed to investigate the effects of the treatment in different doses across the time axis. Systems/Network biology analyses are employed to identify master regulators and their temporal expression in wound healing with and without the presence of mRNA treatments.

Single cell RNAseq: Our group already has invested extensively in creating the largest single cell map of human diabetic wound healing, while we have analyzed and integrated in-house mouse samples with all available relevant mouse studies from us and other groups (Theocharidis et al, 2022, Nature communications 13(1):181, doi: 10.1038/s41467-021-27801-8; Theocharidis et al., 2020, Diabetes 69(10):2157-69, doi: 10.2337/db20-0188; Gay et al., 2020, Sci Adv. 2020; 6(12):eaay3704, doi: 10.1126/sciadv.aay3704; Guerrero-Juarez C F, et al., 2019, Nature communications 10(1):650, doi: 10.1038/s41467-018-08247-x; Haensel D, et al., 2020, Cell reports 30(11):3932-47 e6, doi: 10.1016/j.celrep.2020.02.091; Foster et al., 2021, Proc Natl Acad Sci USA. 118(41), doi: 10.1073/pnas.2110025118; Theocharidis G, et al., 2022, Nature Biomedical Engineering, doi: 10.1101/2021.06.07.447423). To maximally utilize these resources to disentangle the effects of mRNA treatments at the cellular level, scRNAseq data are generated from mouse, pig, and human samples from controls and following investigative treatment at two doses. Optimal time point selection is performed based on the bulk RNAseq studies. In brief, rapid automated tissue dissociation is performed from harvested tissue samples using the automated Singulator 100 (S2 Genomics) to minimize batch variations in gene expression resulting from conventional tissue dissociation procedures (Jovanovich S, et al., editors. Automated processing of solid tissues into single cells or nuclei for sequencing. AGBT 2021; 2020; Florida). Cell viability and number is evaluated with a Luna-FX7 (Logos Biosystems). An estimated 8,000 cells are sequenced per sample. Automated 3′ scRNAseq is performed with the 10× Genomics Connect robotics platform using the vendor's standard protocol with minimal manual steps. We incorporate the following stop points: following GEM generation to photo document quality, prior to cDNA amplification to QC 1 μl of cDNA on a Fragment Analyzer (FA, Agilent), and library QC using the FA. Only libraries passing QC are forwarded for sequencing on an Illumina NovaSeq 6000 and any failed samples are repeated. Single cell data analysis is performed as previously described (Theocharidis G, et al., 2022, Nature communications 13(1):181, doi: 10.1038/s41467-021-27801-8).

Multiplexed Immunofluorescence: 6-plex antibody panels is generated for the Akoya Phenolmager whole slide multispectral scanning platform (Vectra Polaris). Akoya inForm software is utilized to segment the whole slide scans and quantify the markers per region of interest and per cell, providing quantitative data for the temporal and group comparisons.

Spatial Tissue Profiling—Spatial Transcriptomics: To investigate further the effect of mRNA treatments on cell-cell interactions on the tissue microenvironment in situ and to maximize our ability to investigate findings from the bulk, proteomic and single cell studies, Multiplexed error-robust fluorescence in situ hybridization (MERFISH) is performed. This assay enables us to quantify the expression of up to 500 genes in a tissue section at single cell and single molecule resolution. We employ the MERSCOPE instrument (Vizgen, MA) which was first used by the BIDMC Spatial Technologies Unit worldwide in July 2021. In brief, a sectioned tissue block is placed on the MERSCOPE slide and permeabilized overnight using standard techniques that quench autofluorescence. 50 μL MERSCOPE Gene Panel Mix is added to the section and incubated at 37° C. for 40h. The Vizgen Clearing Premix and Proteinase K are used to clear the tissue, while keeping RNA/DNA intact, by following the human skin-specific protocol. Samples are incubated for 24 h or until the tissue section becomes transparent, and then are incubated with DAPI and PolyT Staining Reagent for 15′. Slides are placed into the MERSCOPE and scanned. Up to 1 cm² will be analyzed using a probe set capturing up to 500 targets.

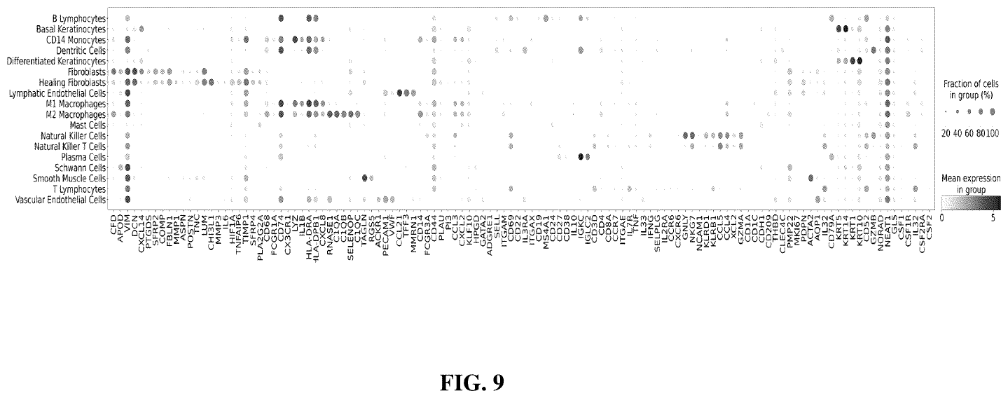

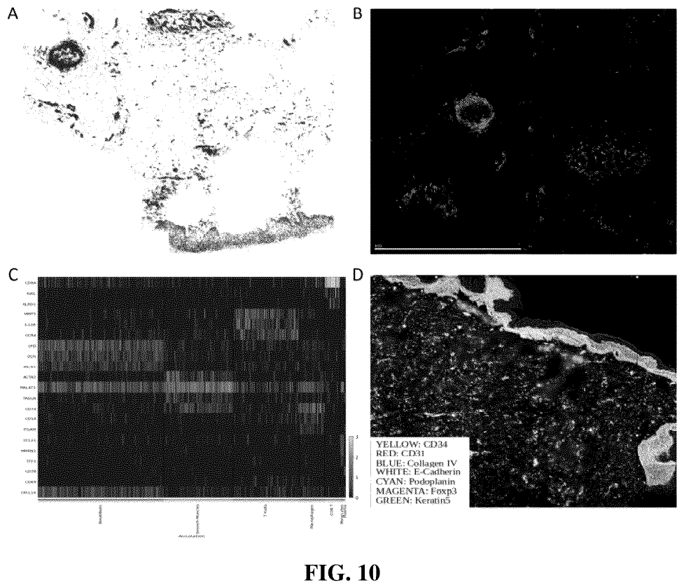

Target Selection: We have already selected 140 targets (FIG. 9) by reanalyzing our internal single cell data for wound healing in humans and mice. Additional targets are incorporated into the assay following the bulk and single cell studies. Spatially distinct regions are detected (Hu J, et al., 2021, Nature methods 18(11):1342-51. doi: 10.1038/s41592-021-01255-8) and scRNAseq data is integrated and registered on the spatial assays using Tangram (Biancalani T, et al., 2021, Nature methods 18(11):1352-62. doi: 10.1038/s41592-021-01264-7). The MERSCOPE datasets are utilized to identify the effects of mRNA treatments. MERSCOPE files are converted to Zarr-AnnData and OME-TIFF format. Extensive QC metrics are extracted (FIG. 10A-C), including the number of cells and RNA molecules detected, sample dimensions, RNA molecule density, and statistics per probe to enable identification of low-performing probes. The multiplexed immunofluorescence assays from serial sections are also overlayed using non-rigid slide registration (Venet L, et al., 2021, Applied Sciences 11(4):1892, doi:10.3390/app11041892). MERSCOPE and overlayed multiplexed IF datasets are further explored with Giotto (Dries R, et al., 2021, Genome Biology 22(1):78. doi: 10.1186/s13059-021-02286-2) to perform differential expression analyses, cell-cell colocalization and communication, as well as to identify spatially variable genes. Spatially distinct regions are detected (Hu J, et al., 2021, Nature methods 18(11):1342-51. doi: 10.1038/s41592-021-01255-8) and scRNAseq data is integrated and registered on the spatial assays using Tangram (Biancalani T, et al., 2021, Nature methods 18(11):1352-62. doi: 10.1038/s41592-021-01264-7). The MERSCOPE datasets are utilized to identify the effects of mRNA treatments on the tissue microenvironment in situ and to maximize our ability to interrogate tissue and to investigate findings from the bulk, proteomic, and single cell studies.

Spatial Proteomics-CODEX: The CODEX platform (Akoya Biosciences) allows the probing of a large number of proteins of interest in a single tissue slide. The section is stained with antibodies that are tagged with unique oligonucleotides and dye-oligonucleotides that function as barcodes and reporters, respectively (FIG. 10D). Following segmentation, spatial datasets are further explored (Hu J, et al., 2021, Nature methods 18(11):1342-51. doi: 10.1038/s41592-021-01255-8; Dries R, et al., 2021, Genome Biology 22(1):78. doi: 10.1186/s13059-021-02286-2) and single cell data are registered on the spatial assays using digital gating, label transfer, and manual curation. Non-rigid slide registration (Venet L, et al., 2021, Applied Sciences 11(4):1892, doi:10.3390/app11041892) is performed to ascertain optimal matching between the CODEX and slides of additional modalities such as H&E or MERSCOPE. All spatial datasets are analyzed in Giotto (Lun A T, et al., 2016, F1000Res. 5:2122, doi: 10.12688/f1000research.9501.2) to identify differentially expressed genes (Lun A T, et al., 2016, F1000Res. 5:2122, doi: 10.12688/f1000research.9501.2), to perform in situ pathway analysis (Dries R, et al., 2021, Genome Biol. 22(1):78, doi: 10.1186/s13059-021-02286-2), to identify spatial domains (Lun A T, et al., 2016, F1000Res. 5:2122, doi: 10.12688/f1000research.9501.2; Hu et al., 2021, Nat Methods 18(11):1342-51, doi: 10.1038/s41592-021-01255-8), and cells co-expressing the genes/proteins of interest as well as spatially variable-genes (Argelaguet R, et al., Genome Biol. 21(1):111, doi: 10.1186/s13059-020-02015-1). Genes affected by the presence of nearby cells (spatial interactions) are identified with a linear model.

Main endpoints, expected results and alternative approaches. The primary endpoint is wound reduction, while secondary endpoints include re-epithelization, granulation and angiogenesis. We expect that the chosen treatments will at least perform as well as in the mouse model. In case of unsatisfactory results, additional mRNAs are considered.

In addition, we expect that the -omics studies will identify all pathways and master genes that are activated or inhibited by the most effective treatments and disentangle their mechanism of action across cell types during wound healing. The use of different in vivo and ex vivo models is a powerful design enabling us to understand conservation of the cellular and molecular mechanics across species and to monitor potential toxicity with high granularity. Of particular interest will be the investigation whether the proposed mRNA treatment will increase the specific fibroblast types that were identified in healing DFU and are shown in preliminary data section.

In addition, the Spatial Technologies Unit supports a wide array of complementary assays, including single nucleus sequencing as well as different additional spatial tissue profiling platforms such as 10× Genomics Visium, Akoya CODEX, NanoString GeoMx DSP, and others, which could be utilized to substitute or complement the proposed assays of the study.

Power analysis. There are very limited data from porcine wound studies, which precludes the ability to perform an a priori power analysis. A recent study in our unit that tested another intervention showed that the effect size in pig studies was similar to, if not better than, mice studies (Theocharidis G, et al., 2022, Nat Biomed Eng., doi: 10.1038/s41551-022-00905-2). We expect that the same will apply for this study and, as a result, 12 wounds per treatment will be sufficient to provide more than 80% power at 5% level of significance. Across all modalities (single cell, spatial), 4+ biological replicates are utilized per group/time point enabling us to achieve high granularity and power in all comparisons. We will have more than 99% power to detect rare cellular populations (less than 1% of cells) with an adequate number of cells per cluster (>100) for at least 10 rare cell populations per group (Davis A, et al., 2019, BMC Bioinformatics 20(1):566. doi: 10.1186/s12859-019-3167-9).

Statistical analysis. Comparisons between groups is performed using the ANOVA test for normally distributed data and the Kruskal-Wallis test for data that are not distributed normally. The General Linear Model is employed for repeated measurement analysis. Bioinformatics: RNA-seq data is processed with current best practices. In brief, data is quality-checked using FastQC (Love et al., 2014, Genome Biol. 15(12):550, doi: 10.1186/s13059-014-0550-8) and reads are aligned to reference genome STAR and a 2-pass approach and quantifying reads with featureCounts (Liao Y, et al., 214, Bioinformatics 30(7):923-30, doi: 10.1093/bioinformatics/btt656). Differential gene expression analysis is performed using DESeq2 (Love et al., 2014, Genome Biol. 15(12):550, doi: 10.1186/s13059-014-0550-8), while ClusterProfiler (v3.12.0) (Yu G, et al., 2012, OMICS 16(5):284-7, doi: 10.1089/omi.2011.0118) is utilized for downstream functional investigations.

Expected results. It is expected that wounds treated with the best two treatment will have >50% healing compared to wounds treated with control bandages or Tegaderm). It is expected that mRNA treatment does not result in increased topical or systemic side effects.

### Example 12. Translational Studies in Human Diabetic Wound Healing to Verify the Therapeutic Potential

Rationale. Although the genes that are involved in wound healing share similarities in mice, pigs and humans, it is very important to confirm that human mRNA can also be effective in human studies. In order to provide such a proof of concept, studies are performed in ex vivo human skin and then in an in vivo humanized skin graft mouse model. We believe that successful completion of these studies will greatly enhance the translational potential of the project, and that the main remaining work before the transition to Phase I/II clinical trials is considered will be conducting toxicology studies.

We have used these two models for other studies in our unit, either unpublished or published (Theocharidis G, et al., 2022, Nat Biomed Eng., doi: 10.1038/s41551-022-00905-2). In addition, a study evaluating LNP-CHI3L1 mRNA and LNP-IL-2 ORF7 mRNA in the humanized skin graft mouse model is described in Example 13 below.

Research Design. Humanized skin graft mice models. Human skin transplantation on immunodeficient mice, which support engraftment, is a valuable study model for investigating mechanisms involved in wound healing. It has been demonstrated in various studies that, after injury, the regeneration of human skin transplanted onto nude mice mimics the process observed in injured patient skin, thus making the establishment of a humanized diabetic wound healing model in our unit a valuable resource for studying the wound healing process in vivo (Ding J, et al., 2017, Methods in molecular biology 1627:65-80. doi: 10.1007/978-1-4939-7113-8_5; Escamez M J, et al., 2004, The Journal of investigative dermatology 123(6):1182-91. doi: 10.1111/j.0022-202X.2004.23473.x. PubMed PMID: 15610532; Leon C, et al., 2020, Genes 12(1), doi: 10.3390/genes12010047). Human skin grafted onto the nude mouse has previously been shown to be able to regenerate after an excisional wound is made through the entire thickness of the graft (Ding J, et al., 2017, Methods in molecular biology 1627:65-80. doi: 10.1007/978-1-4939-7113-8_5).

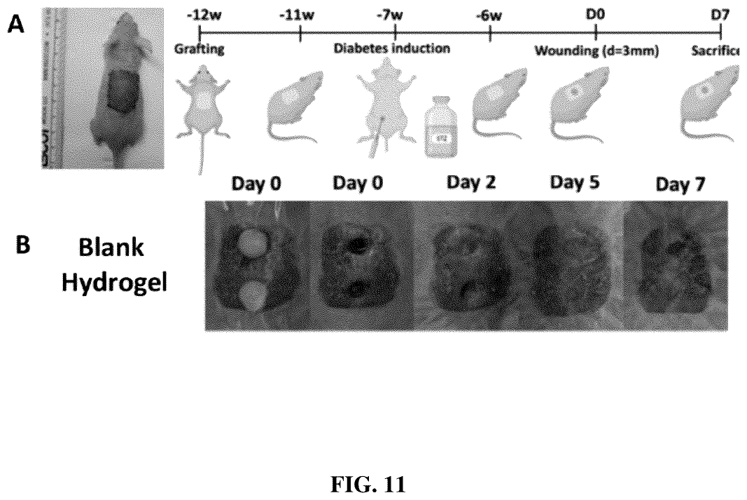

To generate a humanized skin graft mice model, Foxn1nu nude mice of 6 weeks old are used as the recipient animals. These mice are devoid of T cells and suffer from a lack of cell-mediated immunity, making them a model of choice for xenograft studies. Mice dorsal skin is excised with an area where a graft of an equal size of full-thickness or split-thickness human skin is sutured (FIG. 11). Human skin is obtained from IRB-approved discarded human skin from plastic surgeries. Grafted mice are left to heal and accommodate the new tissue for 5 weeks, where the graft will fully mature. Diabetes is induced in stable human skin engrafted mice 5 weeks post-grafting via intraperitoneal injections with streptozotocin (STZ). Mice sustain a hyperglycemic state for 7 weeks prior to wounding experiments. Following the sustained diabetic state period, wounds are inflicted on the transplanted human skin with a 3 mm punch biopsy. Treatment options include the best two mRNA treatments, previously determined by the mice and pig studies. These dressings are then be applied to each individual wound, as well as a blank hydrogel formulation. As bandages can provide sustained release for at least three days, initially changes are performed every three days while the potential of weekly changes is also explored. At days 3, 5 and 10 post-wounding and dressing treatment, mice are euthanized and wound tissue is harvested for histological and molecular analysis. Wound healing is monitored using standardized photography daily during the course of 3, 5 and 10 days. Histological and immunohistochemistry studies are performed as described for the mice studies.

Human ex vivo model of wound healing. To further examine the effects of mRNA treatment on human wound healing, wounds on panniculectomy derived discarded skin are studied. Ex vivo human skin culture wound healing study skin specimens are obtained from BioIVT (Westbury, NY). De-identified samples are provided with removed subcutaneous adipose tissue in sterile PBS at 4° C. on the day of surgery and are immediately processed. A 6 mm biopsy punch is used to gently punch the skin's epidermis partially penetrating into the dermis to create a wound. The best performing mRNA treatment and a Blank Hydrogel formulation are topically applied, and the skin specimens are placed dermis side down onto the culturing dishes and incubated at 37° C., 5% CO2. Healing is monitored for four days using the techniques as in a recent study that involved ex vivo human skin in our unit.

Transcriptomic and proteomic analysis. Similar -omic techniques to those described in Example 9 above are employed to evaluate in vivo and ex vivo effects of mRNA treatment in human tissue.

Main endpoints, expected results. The main endpoint will be the same as with the mice studies described in Example 9, namely reduction in the wound size and the secondary endpoints include re-epithelialization, granulation tissue formation and angiogenesis. We expect that for the chosen two doses the results will be similar or better to the ones that will be observed in mice and as presented. In addition, we expect that transcriptomic analysis will show that the application of the mRNA treatments will have a very similar profile to that obtained in DFU patients who healed the ulcers and as presented in Example 7.

Power analysis. Studies in our unit that employed another intervention showed that when comparing humanized mice wounds treated with control versus those treated with active treatment, we had a pooled standard deviation of 29 and a difference 30, which indicates that 16 wounds in each group will allow us to detect a difference in wound healing improvement with more with 80% power and 5% level of significance.

Statistical analysis. Similar to the statistical analysis that is proposed for mice studies in Example 9.

Alternative approaches. The recent successful completion of studies that employed both humanized mice and ex vivo human skin provides evidence that we can complete the proposed experiments without any major difficulties. Different types of immunocompromised mice and human skin specimens from other anatomical sites, including skin from amputated legs, may also be used.

Biological variables: Both female and male mice and pigs are used. Although current information indicates that there are no sex differences in impaired wound healing in human diabetes, our research design will allow us to evaluate the efficacy of proposed treatments in both sexes and identify possible differences.

### Example 13. Evaluation of LNP-CHI3L1 mRNA and LNP-IL-2 ORF7 mRNA in a Humanized Skin Graft Mouse Model with Streptozotocin (STZ)-Induced Diabetes

### Methods

As our previous studies showed that the highest potential was shown for LNP-CHI3L1 mRNA and LNP-IL-2 ORF7 mRNA treatment, we further validated their potency in the diabetic humanized skin graft mouse model. In this model, human skin is grafted to the back of athymic nude mice, and diabetes is induced with streptozotocin (STZ). We also specifically selected eGFP mRNA as the negative control mRNA to study the delivery system and cellular uptake in the downstream wound analysis. Three circular biopsy punch (3-mm in diameter each) full-thickness wounds were created on STZ-induced diabetic humanized mice dorsum. LNP-mRNA-loaded alginate bandages were placed on the wounds immediately after their creation. A waterproof, transparent wound dressing (Tegaderm™) was placed on top for the protection of the gel and wounds.

### Results

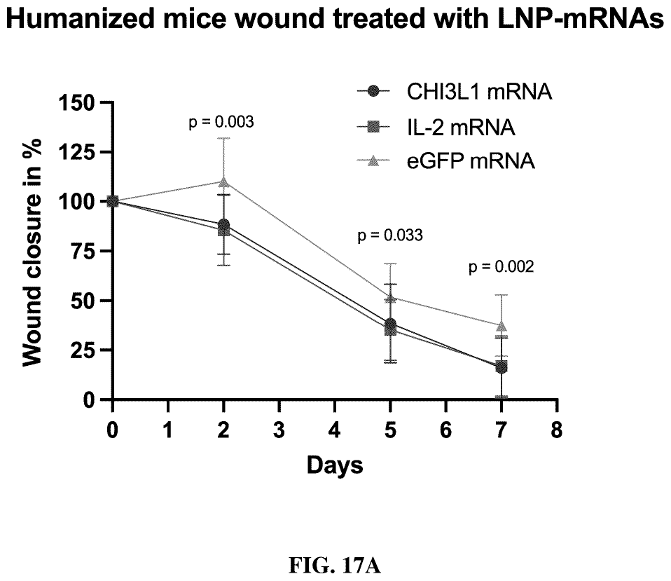

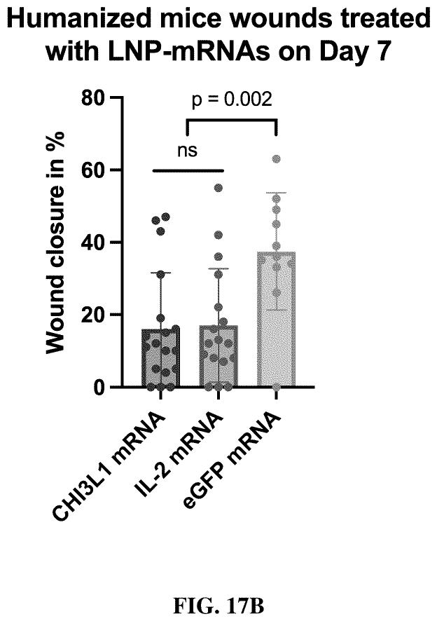

Both CHI3L1 mRNA and IL-2 ORF7 mRNA treatment improved wound healing when compared to the eGFP control treatment as early as two days after injury, and the difference was sustained until study completion on Day 7 (FIG. 17A). Both LNP-Chi3L1 mRNA and LNP-IL-2 ORF7 mRNA were equally efficient in accelerating mouse dorsal wound healing, resulting in a wound size of ˜16% of the original size on Day 7, whereas eGFP mRNA treatment resulted in a wound size of ˜37% of the original size on Day 7, on average. (FIG. 17B). These results demonstrate the efficacy of delivered CHI3L1 and IL-2 ORF7 mRNA sequences in wound healing of human skin. In addition, these results from grafted human skin are consistent with the results found in our db/db mice study, demonstrating the efficacy of the db/db mouse model in predicting wound healing activity in human skin. Notably, the healing wounds on grafted human skin showed no sign of infection or side effects after being treated with CHI3L1, IL-2 ORF7, or eGFP mRNAs. These results demonstrate that the LNP-mRNA delivery system is also effective in human skin, and further support the use of Chi3L1 mRNA and IL-2 ORF7 mRNA for the treatment of diabetic foot ulceration.

### Example 14. Targeting Delivery of Modified mRNA to Specific Cell Types Through Chemical Modification of LNPs

The CHI3L1 gene is found overexpressed in a unique subset of fibroblasts and is highly associated with DFU healing. Targeting delivery of modified mRNA in specific cell types, such as fibroblast, is our next step based on the accelerated wound closure rate induced by successful LNP-CHI3L1s delivery. To achieve LNP-mRNAs specific target cells, the LNP surface is modified to be internalized by a specific target cell type. The surface of LNPs is chemically modified with fibroblast-associated peptides or ligands to enhance the delivery of LNP-CHI3L1 mRNA. LNP-mRNA is formulated by mixing ionizable lipids, PEG-lipid, cholesterol, and phospholipids in an acidic environment. The selected peptide sequence or ligands are incorporated into the PEG-lipid in the LNP-mRNA system. The efficacy of peptide conjugation is determined by chromatography or western blot. Before employing the modified LNP-CHI3L1 mRNA on mouse wounds, the specificity of targeted cell delivery of modified LNP is validated in vitro. Experiments are carried out using different cell lines, including keratinocytes, endothelial cells, fibroblasts, and polarized macrophages, with eGFP mRNA as reporter genes to determine cellular uptake rates. Three peptides or ligands are selected to start with and adjusted based on the uptake rates.

### Example 15. Evaluation of LNP-FGF2, LNP-IL-2, LNP-CHI3L1, and LNP-IL-17A mRNA in a db/db Mouse Model

### Methods

The mRNAs and bandages used in these studies were the same as those described in Example 7 above, except that the IL-2 ORF7 mRNA was replaced with an mRNA encoding full length wildtype mouse IL-2 (SEQ ID NO: 14), designated here as “IL-2”. The new IL-2 mRNA had the same general structure as the mRNAs described in Example 7, i.e., full substitution of both Pseudo-U and 5-Methyl-C, a 5′ cap structure, a 5′ UTR, a 3′ UTR, a polyA tail, and it was not codon optimized. The IL-2 coding region in the IL-2 mRNA was encoded by SEQ ID NO: 13.

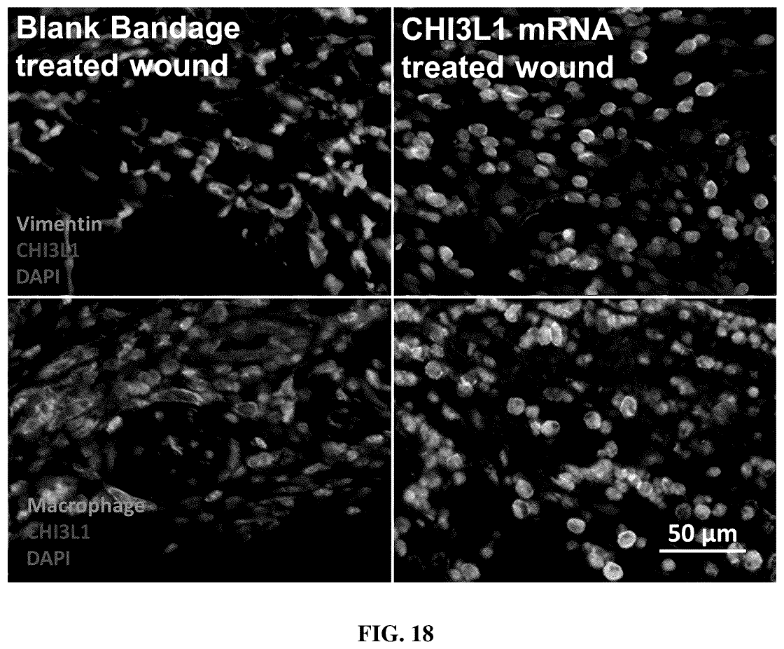

We co-stained diabetic mouse wound sections treated with either blank bandages or LNP-CHI3L1-mRNA releasing bandages for three days for the mesenchymal marker Vimentin, a macrophage specific marker (antibody RM0029-11H3) and CHI3L1 (FIG. 18).

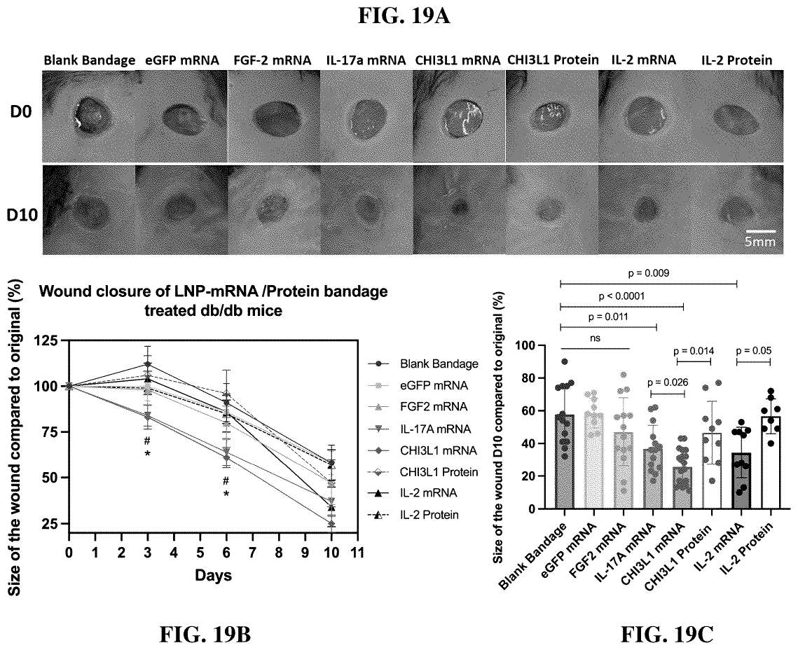

We then evaluated the efficacy of LNP-FGF2, LNP-IL-2, LNP-CHI3L1, and LNP-IL-17A mRNA treatment in diabetic db/db mice. This is the same db/db mouse model described in Example 7 above. Two 6-mm punch biopsy full-thickness wounds were created on the dorsum of 16-week-old diabetic db/db mice (FIG. 19A). Macroporous bandages loaded with 4 μg of LNP-FGF2, LNP-IL-2, LNP-CHI3L1, and LNP-IL-17A mRNA were topically applied on the wounds for 10 days. Blank bandages and bandages loaded with eGFP, CHI3L1 protein and IL-2 protein were also employed as additional controls. The wound size was measured every three days.

### Results

We found enhanced co-localization of CHI3L1 with both mesenchymal cells and macrophages in the treated wounds indicating that these cell types could be taking up the LNPs and successfully translating the CHI3L1 mRNA to protein. In the blank treated wounds, minimal CHI3L1 co-localization was observed. See FIG. 18.

Repeated measurement analysis using the general linear model showed that there were no differences in wound healing between the blank bandage and the other three controls (eGFP and proteins CHI3L1 and IL-2). All four mRNA treatments (FGF2, IL-2, CHI3L1, and IL-17A) performed better than the blank bandage during the whole period of the experiment (FIG. 19B). CHI3L1 and IL-17A performed better than IL-2 and FGF2 while CHI3L1 tended to performed better than IL-17A. On Day 10, wound reduction was similar between blank, eGFP, IL-2 ORF7 protein, CHI3L1 protein and FGF2 mRNA, lower in IL-17A and IL-2 mRNA and the lowest in CHI3L1 mRNA treatment (FIG. 19C). Collectively, these findings indicate that IL-2 and CHI3L1 mRNA loaded LNPs are the most efficient in promoting diabetic wound repair and outperform their respective recombinant proteins treatment, which are most possibly degraded in the highly proteolytic wound microenvironment. They also imply that CHI3L1 and IL2 act during different stages of wound healing, with the latter exerting its beneficial effect mostly after day 6, which coincides with the proliferation phase. In contrast, CHI3L1 is more effective earlier, at the late inflammation phase.

### Example 16. Evaluation of LNP-CHI3L1 mRNA in a Human Skin Xenograft Diabetic Mouse Model, and Single-Cell RNA-Seq Analysis of Treated Wounds

### Methods

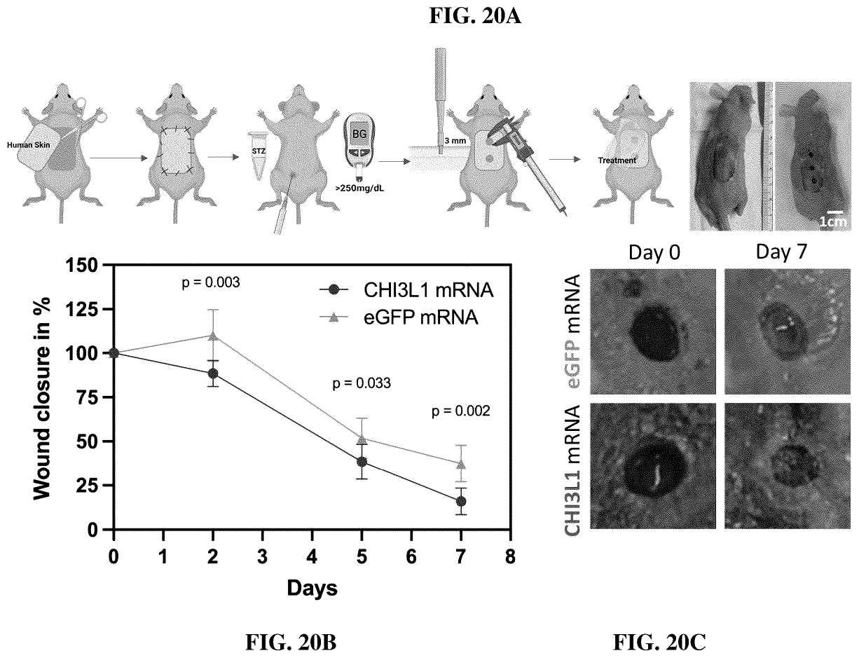

We completed a set of experiments that involved LNP-CHI3L1 mRNA application on wounds of human skin xenografts transplanted onto diabetic immunocompromised mice. (FIG. 20A). This model is the same human skin xenograft model described in Example 13 above. We also specifically selected eGFP mRNA as our control mRNA to study our delivery system and cellular uptake in the downstream wound analysis.

To better understand specific cell functions and gain deeper insights into the mechanisms of faster wound healing, we then performed single-cell RNA-seq analysis on LNP-CHI3L1-mRNA treated human skin xenografted diabetic mice wounds and on LNP-eGFP-mRNA treated wounds at day 3.

### Results

We found improved wound healing with CHI3L1 mRNA treatment, when compared to the eGFP control, as early as two days after injury and the difference was sustained until study completion on Day 7 (FIG. 20B, 20C). LNP-CHI3L1 mRNA resulted in wound size of ˜16% of their original size, whereas eGFP mRNA yielded wound size of ˜37% on average (FIG. 20B). We have therefore successfully validated the efficacy of delivered CHI3L1 mRNA sequence in this grafted human skin model and our results are consistent with the ones observed in the db/db mice study (FIG. 19). Notably, the healing wounds on grafted human skin showed no untoward effects after being treated with CHI3L1, or eGFP mRNAs.

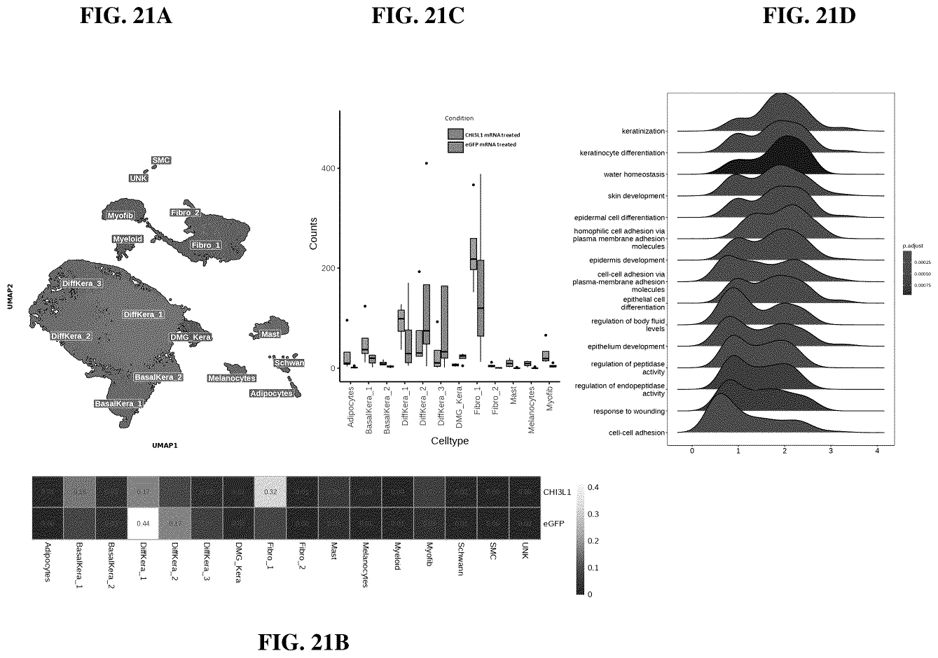

For the single-cell RNA-seq analysis, Uniform Manifold Approximation and Projection (UMAP) and cell annotation revealed multiple clusters encompassing all expected cell types: fibroblasts, myeloid cells, keratinocytes, mast cells, melanocytes and keratinocytes (FIG. 21A). To compare the two treatments, we generated a heatmap of the proportion of cell types per condition. Enrichment (4-fold) of the Fibro_1 subcluster has found in LNP-CHI3L1-mRNA treated wounds when compared to LNP-eGFP-mRNA treated wounds (FIG. 21B). Furthermore, when investigating which cells express the CHI3L1 gene, we noticed a high expression in the Fibro_1 subcluster (FIG. 21C) pointing to fibroblasts as a primary cell for uptake and effective expression of the delivered mRNA. Moreover, in the Pathway barplot of the Fibro_1 cluster, biological pathways that are significantly affected in this cell type include response to wounding, as well as the skin/epithelium development and cell adhesion pathways highlighting a key role for these cells in integral repair processes (FIG. 21D).

### Example 17. Evaluation of LNP-CHI3L1-mRNA in an Ex Vivo Human Skin Model

### Methods

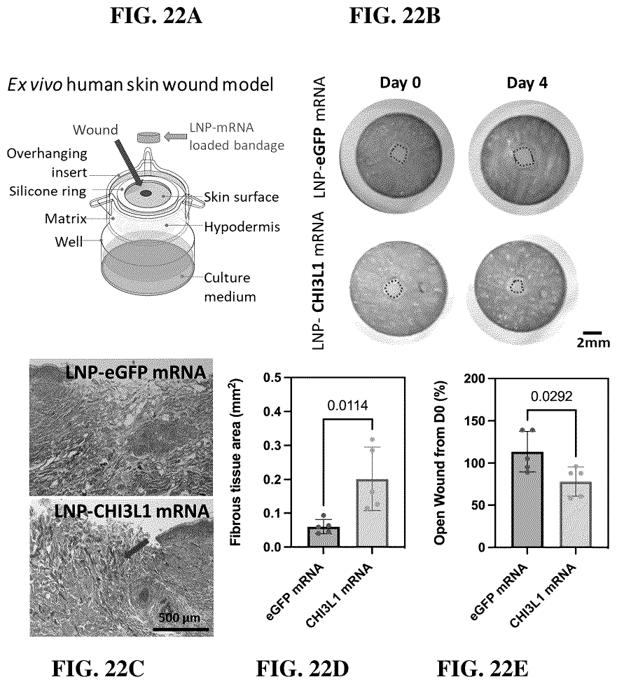

To further assess the efficiency of mRNA treatment on human wounds, we obtained commercially available discarded human skin with an already existing wound in the middle. This 3D culture system maintains the epidermis at an air-liquid interface and provides reproducible and physiologically relevant wounds to study different treatment conditions (FIG. 22A). We treated existing wounds either with LNP-eGFP-mRNA, or LNP-CHI3L1-mRNA for 4 days.

### Results

Our results consistently showed that LNP-CHI3L1-mRNA improves wound healing (FIGS. 22B and 22D left) and increases fibrous-granulation tissue area in the wound, which means that it stimulated the wound healing process (FIGS. 22C and 22D right).

### Example 18. Evaluation of LNP-CHI3L1-mRNA in Porcine Dermal Fibroblasts In Vitro

### Methods

We explored whether the LNP-CHI3L1-mRNA could be functional in another species by delivering the particles to porcine dermal fibroblasts in vitro.

### Results

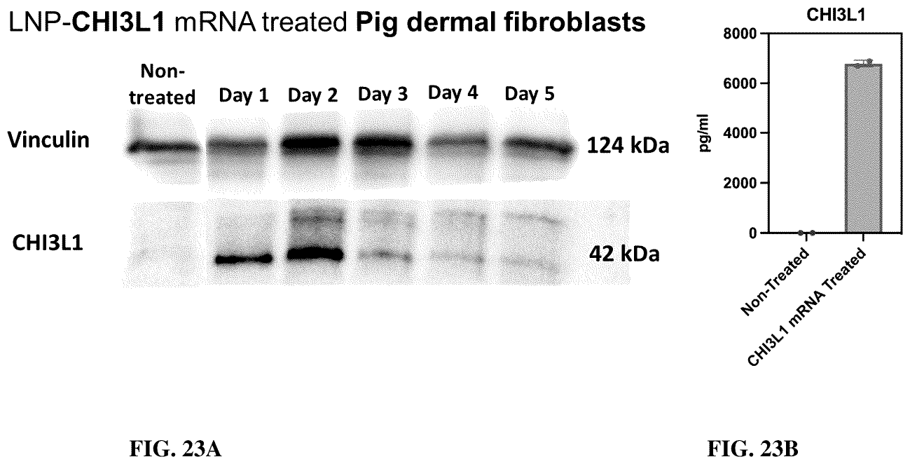

A single dose of LNP-CHI3L1-mRNA (500 ng/ml) induced elevated expression of CHI3L1 on days 1, 2 and 3, peaking on day 2 as illustrated by western blot analysis (FIG. 23A). We corroborated that the protein was indeed secreted by the cells, as quantitated by ELISA of the conditioned media (FIG. 23B).

The following relates to the treatment of ulcerative conditions using acetylcholinesterase inhibitors. Metrifonate ((2,2,2-trichloro-1-hydroxyethyl) dimethylphosphonate) is one example of such an organophosphate acetylcholinesterase inhibitor. Metrifonate is currently being used in various parts of the world as an oral treatment of urinary schistosomiasis at a single dose of 10 mg/k. It has also been clinically tested for the treatment of mild-to-moderate Alzheimer's disease, at oral doses of 30-60 mg daily for periods up to 26 weeks and it was found to have a beneficial effect but was not introduced to clinical practice due to rare side effects such as neuromuscular dysfunction and respiratory failure. Nonetheless, other acetylcholinesterase inhibitors, such as donepezil and rivastigmine are currently employed for the management of Alzheimer's disease. In certain aspects, acetylcholinesterase inhibitors such as metrifonate are suitable for the treatment of DFU wounds.

### A. Wound Dressings

Metrifonate can be incorporated into a wound dressing using an alginate hydrogel. This wound dressing can be configured for time release during the inflammatory and proliferation phases of wound healing. In certain aspects, the disclosure relates to a wound dressing comprising an alginate hydrogel having a therapeutic amount of an acetylcholinesterase inhibitor distributed therein for release onto an ulcerative wound during a delivery period. In some aspects, the wound dressing comprises an absorbent material. In some embodiments, the acetylcholinesterase inhibitor is selected from the group consisting of donepezil, revastigmine, galantamine and tacrine. In certain embodiments, the acetylcholinesterase inhibitor comprises metrifonate.

In certain implementations, the metrifonate is electrostatically bound within the alginate hydrogel until release onto the wound. In one implementation, the alginate hydrogel can comprise an agent that inhibits release of the metrifonate over a selected period of time. This agent can comprise laponite that electrostatically inhibits release from the wound dressing. The metrifonate can also be incorporated into polymer microspheres that degrade over time to release from the wound dressing over a selected time period. In some embodiments, the polymer microspheres comprise poly lactide-co-glycolide (PLG).

Preferred embodiments relate to wound dressings in which time release of a therapeutically effective dose of metrifonate can be delivered to a wound. An alginate hydrogel, configured to form a bandage, is able to release a therapeutically effective amount of metrifonate over a selected time. In certain aspects, the wound dressing comprises the metrifonate in an amount sufficient to result in an accumulated concentration of metrifonate in the wound at less than 1000 μg/ml. In some implementations, the wound dressing comprises a sufficient amount of an acetylcholinesterase inhibitor to increase the wound closure rate when compared to an ulcerative wound that is not treated with the acetylcholinesterase inhibitor. In some embodiments, the amount of the acetylcholinesterase inhibitor is sufficient to increase the re-epithelialization of the wound and/or blood vessel density of the wound relative to an ulcerative wound that is not treated with the acetylcholinesterase inhibitor. The amount of the acetylcholinesterase inhibitor may also be sufficient to increase one or more of total mast cells in the wound and degranulated mast cells in the wound relative to an ulcerative wound that is not treated with the acetylcholinesterase inhibitor.

In some aspects, the amount of the acetylcholinesterase inhibitor in the wound dressing is sufficient to decrease the expression of genes selected from the group consisting of Transforming growth factor beta 1 (TGFB1), alpha-2 actin (ACTA2), interleukin 1B, (IL-1B), Matrix metalloproteinase 9 (MMP9) and Neutrophil gelatinase-associated lipocalin (NGAL), relative to an ulcerative wound that is not treated with the acetylcholinesterase inhibitor. In some embodiments, the wound dressing comprises the acetylcholinesterase inhibitor in an amount that is sufficient to decrease the levels of neutrophil marker NIMP-R14+ cells in the wound and/or increase the number of macrophage marker F4/80+ cells in the wound, relative to an ulcerative wound that is not treated with the wound dressing comprising the acetylcholinesterase inhibitor. In certain embodiments, the amount of the acetylcholinesterase inhibitor in the wound dressing is sufficient to increase the expression of one or more genes selected from the group consisting of cholinergic receptor muscarinic 1 (Chrm1), Chrm2, Chrm3, and cholinergic receptor nicotinic alpha (Chrna7), relative to an ulcerative wound that is not treated with the wound dressing comprising the acetylcholinesterase inhibitor. In one embodiment, the acetylcholinesterase inhibitor activates cholinergic receptor muscarinic 1 (Chrm1).

In some implementations, wound dressings of the disclosure are formulated to be applied to the ulcerative wound during an inflammatory phase of the ulcerative wound. In some aspects, the wound dressings further comprise an adhesive layer to attach the wound dressing to skin surrounding the ulcerative wound on a foot of a subject.

### B. Methods of Treating Wounds

In certain aspects, the disclosure relates to a method of treating a diabetic ulcer wound in a subject, comprising applying a wound dressing to the diabetic ulcer wound of the subject, wherein the wound dressing comprises a therapeutically effective amount of an acetylcholinesterase inhibitor, and an alginate hydrogel configured to release a therapeutic dose of the acetylcholinesterase inhibitor onto the wound for a delivery period. In some embodiments, the acetylcholinesterase inhibitor is selected from the group consisting of donepezil, rivastigmine, gelantamine and tacrine. In certain embodiments, the acetylcholinesterase inhibitor comprises metrifonate ((2,2,2-trichloro-1-hydroxyethyl) dimethylphosphonate).

In some implementations of the methods of treating wounds of the disclosure, the alginate hydrogel includes an agent that electrostatically inhibits release of metrifonate during the delivery period. In certain embodiments, the agent comprises laponite. In some embodiments, the metrifonate used in a method of treating wounds is encapsulated in a plurality of polymer microspheres. In some embodiments, the polymer microspheres comprise poly lactide-co-glycolide (PLG).

In certain aspects, the method of wound treatment comprises application of acetylcholinesterase inhibitor to the diabetic ulcer wound in an amount sufficient to result in an accumulated concentration of metrifonate in the wound at below 1000 μg/ml. In some implementations, the application of an acetylcholinesterase inhibitor is sufficient to increase the wound closure rate when compared to an ulcerative wound that is not treated with the acetylcholinesterase inhibitor. In some embodiments, the amount of the acetylcholinesterase inhibitor applied to the wound is sufficient to increase the re-epithelialization of the wound and/or blood vessel density of the wound relative to an ulcerative wound that is not treated with the acetylcholinesterase inhibitor. The amount of the acetylcholinesterase inhibitor applied may also be sufficient to increase one or more of total mast cells in the wound and degranulated mast cells in the wound relative to an ulcerative wound that is not treated with the acetylcholinesterase inhibitor.

In some aspects, the amount of the acetylcholinesterase inhibitor applied to the diabetic ulcer wound is sufficient to decrease the expression of genes selected from the group consisting of Transforming growth factor beta 1 (TGFB1), alpha-2 actin (ACTA2), interleukin 1B, (IL-1B), Matrix metalloproteinase 9 (MMP9) and Neutrophil gelatinase-associated lipocalin (NGAL), relative to an ulcerative wound that is not treated with the acetylcholinesterase inhibitor. In some embodiments, the method of treating wounds comprises applying the acetylcholinesterase inhibitor in an amount that is sufficient to decrease the levels of neutrophil marker NIMP-R14+ cells in the wound and/or increase the number of macrophage marker F4/80+ cells in the wound, relative to an ulcerative wound that is not treated with the wound dressing comprising the acetylcholinesterase inhibitor. In certain embodiments, the amount of the acetylcholinesterase inhibitor applied to the wound is sufficient to increase the expression of one or more genes selected from the group consisting of cholinergic receptor muscarinic 1 (Chrm1), Chrm2, Chrm3, and cholinergic receptor nicotinic alpha (Chrna7), relative to an ulcerative wound that is not treated with the wound dressing comprising the acetylcholinesterase inhibitor. In one embodiment, the acetylcholinesterase inhibitor activates cholinergic receptor muscarinic 1 (Chrm1).

In some implementations, wound dressings are applied to the diabetic ulcerative wound during an inflammatory phase of the ulcerative wound. In some aspects, the wound dressings further comprise an adhesive layer to attach the wound dressing to skin surrounding the diabetic wound on a foot of a subject, for example. Other anatomic sites that are prone to develop such ulcerative conditions can also be treated with the wound dressings and methods of application and delivery described herein.

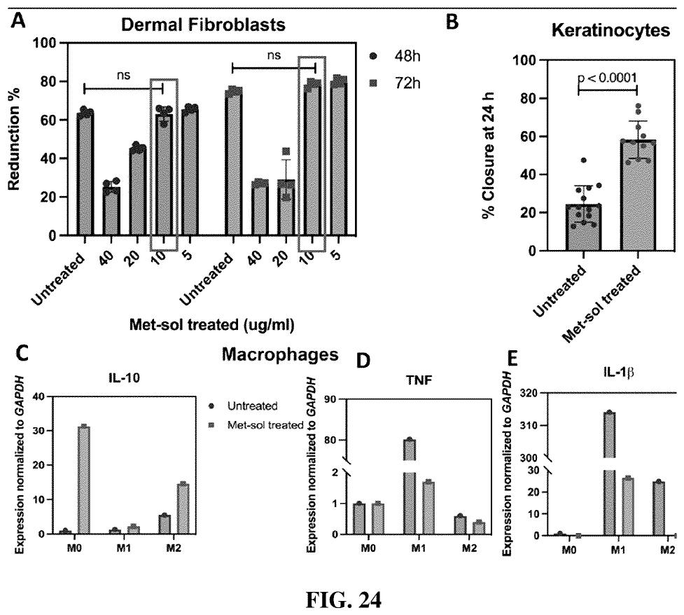

Following the in silico prioritization of metrifonate as a key compound potentially able to positively affect wound healing, we first set out to determine whether addition of metrifonate in tissue cultures of important cells during wound healing, i.e. fibroblasts, keratinocytes and macrophages influences key cell functions. The fibroblast metabolic activity was monitored with the Alamar Blue assay over three days after treatment with a range of metrifonate concentrations and found that anything over 10 ug/ml began to negatively affect viability. This concentration was selected for all subsequent experiments (FIG. 24A). In a wound healing scratch assay experiment, treatment of keratinocytes with metrifonate significantly accelerated migration to facilitate wound closure after 24 hours (FIG. 24B). Furthermore, THP-1 derived macrophages polarized to MO, M1 and M2 phenotypes displayed a notable increase in anti-inflammatory and pro-regenerative gene expression of IL10 (by 30-fold in MO and 10-fold in M2) and a remarkable downregulation of pro-inflammatory cytokines TNF and IL1b in M1 cells, by 40-fold and 12-fold respectively (FIG. 24C-E). Taken together, these findings indicate that metrifonate exerts multiple beneficial effects that promote integral processes in wound healing, such as expedited re-epithelialization and excessive inflammation attenuation.

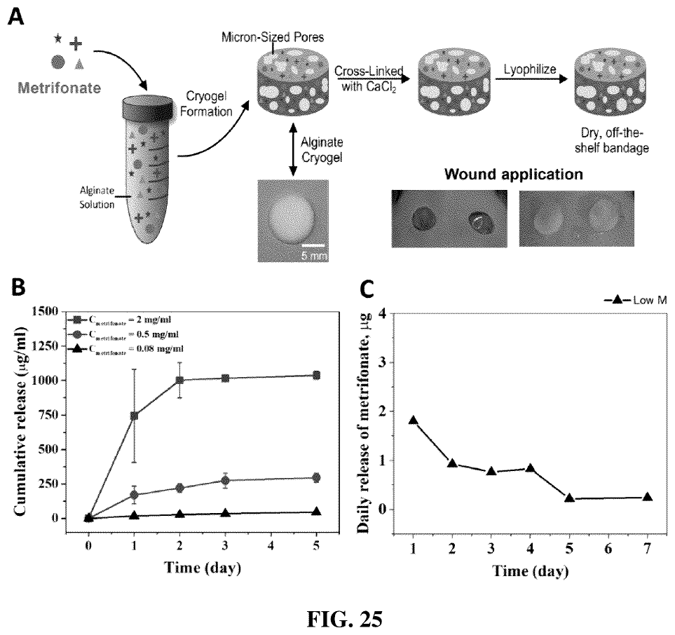

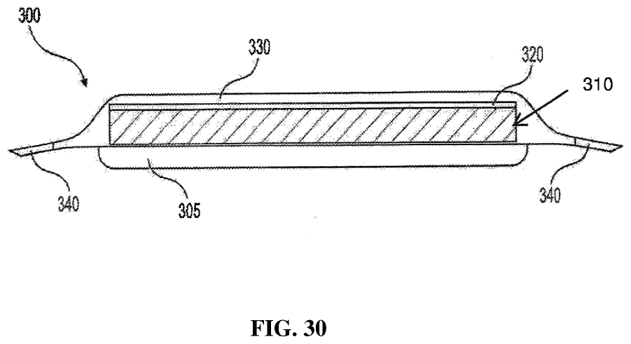

A wound dressing such as a bandage using an alginate, an FDA approved polysaccharide widely used to fabricate commercial wound dressings (e.g., ALGICELL®), can be configured to provide a sustained (>7 days) release of different metrifonate concentrations after placement on wounds. Metrifonate was incorporated into alginate solutions used for fabricating the bandage (FIG. 25A), and the resulting solution placed into pre-formed molds, frozen, and lyophilized to create micron-sized pores in the bandage as illustrated. The cryogels were then cross-linked with calcium chloride, and re-lyophilized to form dry, off-the-shelf bandages that can be stored and are appropriately sized to cover biopsy punch inflicted wounds. Further details regarding the fabrication of this wound dressing material can be found in U.S. Pat. Nos. 10,016,524 and 10,391,136, the entire contents of each of these patents being incorporated herein by reference. The shelf life of these bandages is on the order of months when kept frozen under a dry atmosphere. As shown in FIG. 30 this wound dressing 300 can have a backing layer 320 on which the cryogel 310 is positioned at a central portion relative to an adhesive layer 330 and an adhesive 340 is located at a peripheral region on the adhesive layer to contact the skin of the patient surrounding the wound. A removable strip of material can cover the adhesive and the cryogel prior to removal of the strip for placement over the wound 305.

In order to provide a more continuous and sustained release over seven days or longer, Laponite was mixed with clay nanodisks with the metrifonate/alginate solution (FIG. 25A) prior to fabricating the bandages. Metrifonate associates with the laponite through electrostatic interactions and, as a result, the initial release is diminished and the duration of release extended for a selected. See for example, Koshy S T, Zhang D K Y, Grolman J M, Stafford A G, Mooney D J. Injectable nanocomposite cryogels for versatile protein drug delivery. Acta Biomater. 2018; 65:36-43. Epub 2017/11/13. doi: 10.1016/j.actbio.2017.11.024. PubMed PMID: 29128539; PMCID: PMC5716876, the entire contents of which is incorporated herein by reference. An acetylcholinesterase (AChE) activity assay kit can be used from day 1 to day 7 to determine the release of metrifonate from alginate hydrogels. Each metrifonate loaded hydrogel was first submerged in 500 microliter phosphate buffer saline (PBS). The supernatant from high dose (initial load: 2 mg/ml), medium dose (initial load: 0.5 mg/ml), and low dose (initial load 0.08 mg/ml) metrifonate hydrogels were collected for AChE activity detection from day 1 to day 7 (FIG. 25B). The AChE activity assay kit enabled ing of the AChE inhibitor concentration. The cumulative release from high dose metrifonate hydrogel (HD Met-gel) and medium dose metrifonate hydrogel (MD Met-gel) reached an equivalent point at day 3 with a concentration of 1000 g/ml and 250 g/ml, respectively. To better illustrate drug release from low dose metrifonate hydrogel (LD Met-gel), daily metrifonate release was determined in micrograms (FIG. 25C).

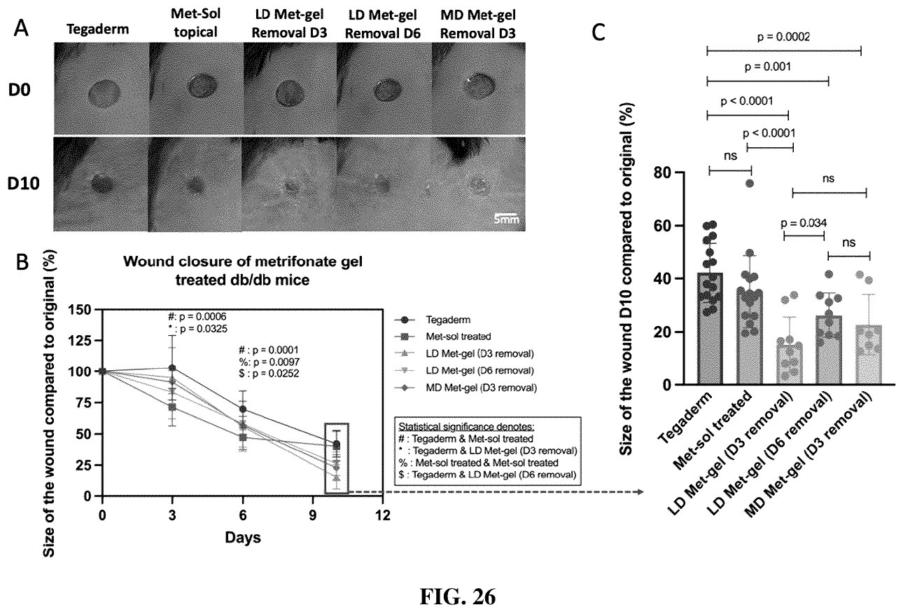

To evaluate the effect of metrifonate releasing bandages in vivo, an established model of impaired diabetic wound healing was selected using db/db mice (FIG. 26A). According to the release study, the release of metrifonate reached to equivalent levels at day 3 (D3). Thus, metrifonate hydrogels were removed or/replaced every 3 days post-wounding. Tegaderm and topical metrifonate solution (Met-sol) were two control groups. The low dose (LD) Met-gel removed at D3 was the most efficient in accelerating mouse dorsal wounds healing initiating the re-epithelialization process (FIG. 26B-C). LD Met-gel removed at D6 and MD Met-gel removed at D3 also improved wound healing to a lesser degree while application of soluble metrifonate (TTMet-sol) had no TH effect. These results indicate that application of low doses of metrifonate during the early stages of wound healing, coinciding with the inflammatory phase improve impaired diabetic wound healing.

When biologic agents are delivered without control over the rate of release, large doses are frequently required to stimulate a therapeutic effect, and the dosing must be frequently repeated; these high, repeated doses, beyond the waste and added expense, can lead to increased toxicity or undesirable side effects. The design of hydrogels can be utilized to provide for sustained drug delivery. See for example, Li J, Mooney D J. Designing hydrogels for controlled drug delivery. Nat Rev Mater. 2016; 1(12). Epub 2016/12/01. doi: 10.1038/natrevmats.2016.71. PubMed PMID: 29657852; PMCID: PMC5898614. the entire contents of which is incorporated herein by reference. A bandage-type macroporous alginate gel, which has been previously developed for drug delivery that can be placed directly over the wound, can be configured to release metrifonate directly into the site. A hydrogel was specifically selected with different metrifonate loading concentrations and different treatment periods to investigate the optimal dosage and application time. The metrifonate can be electrostatically absorbed into the laponite and then loaded into previously developed alginate bandage, using the same techniques that are described. The release of the metrifonate can be altered by different concentrations of laponite and measured through AChE activity assay for all different loading concentrations.

As metrifonate has been demonstrated to improve wound healing in diabetic mice, additional studies can be performed in db/db mice using a range of doses to identify the preferred two doses tested in a pig model. The same methodology that was employed for the studies described above can be employed. More specifically, a dose-dependent efficacy study can be performed in the post-wounding model to determine the minimum efficacy dose. Plasma and local tissue samples can be analyzed for drug exposure, thus providing correlations of exposure and response. Two 6 mm full-thickness skin wounds can be formed on 16-week old male and female db/db mice as previously described in Tellechea A, Kafanas A, Leal E C, Tecilazich F, Kuchibhotla S, Auster M E, Kontoes I, Paolino J, Carvalho E, Nabzdyk L P, Veves A. Increased skin inflammation and blood vessel density in human and experimental diabetes. The international journal of lower extremity wounds. 2013; 12(1):4-11. Epub 2013/03/01. doi: 10.1177/1534734612474303. PubMed PMID: 23446362; PMCID: PMC3688045. Therapeutic effect on wound closure, including re-epithelialization and granulation is analyzed as previously published. Wounds are harvested after animal sacrifice and used in the mechanistic study.

The treatment can be administered as follows, according to the different study groups: i) Five different doses of metrifonate-hydrogel loaded formulation, ranging from 80 μg/ml up to 2000 μg/ml and a blank hydrogel formulation are topically applied to the wounds of the mice for 10 days after wounding (from day 1 to day 10). As dressings can provide sustained release for at least three days, initially changes can be performed every three days while the potential of weekly changes can be examined with the chosen two best doses. At either day 3, 5 or 10 post-wounding and dressing treatment, mice are euthanized and wound tissue is harvested for histological and molecular analysis. Wound healing is monitored using standardized photography daily during the course of 3, 5 and 10 days. Further analysis is performed: 1) H&E staining to evaluate inflammatory or immunological response to the treatment (in vivo toxicity); 2) Immunohistochemical staining to assess angiogenesis (CD31 and VWF); 3) immunofluorescent staining to evaluate the M1/M2 macrophage status. 4) protein levels of factors involved in wound healing, such as VEGF, MMP-9, IL-6, KC, NEP, VEGFR1/2, PDGF, TNFα, SP, SDF1α, MMP-2 and NF-kB. The expression can be evaluated in both unwounded skin (day 0) and wounded skin (days 3, 5 and 10) by qRT-PCR and western blot/immunohistochemistry using standard techniques; 5) serum protein levels of inflammatory cytokines and biochemical markers of endothelial function will be measured using a Luminex 200 apparatus (Luminex, Austin, TX); 6) drug concentration: tissues and plasma samples can be processed and analyzed by LCMS/MS to quantify the drug concentrations.

This can demonstrate wound reduction with each tested treatment and also establish a degree of re-epithelization, granulation and angiogenesis. Additional features can include the number of inflammatory cell infiltration, collagen deposition, skin hemoglobin oxygen saturation and M1/M2 macrophage ratio.

As metrifonate treatment of DM mice accelerates wound healing. There can be differences in the efficacy of each tested dose and time of delivery, e.g. low dose of metrifonate in the early stages of wound healing will be more beneficial as it can stage a robust acute inflammatory response, which facilitates progression to the next phase, the proliferation phase. This provides valuable information regarding the mode of action and differences in the gene expression and pathways that are affected by each metrifonate dose and delivery timing. In formulations in which laponite leads to greatly varying release rates, the metrifonate can instead be encapsulated into poly(lactide-co-glycolide) (PLG) microspheres using a standard double emulsion technique. Metrifonate-encapsulating PLG microspheres can then be incorporated into the alginate bandage; and release from the PLG can be controlled by its degradation kinetics, and regulate the overall kinetics of release of metrifonate from the bandages. See Molavi F, Barzegar-Jalali M, Hamishehkar H. Polyester based polymeric nano and microparticles for pharmaceutical purposes: A review on formulation approaches. Journal of controlled release: official journal of the Controlled Release Society. 2020; 320:265-82. Epub 2020/01/22. doi: 10.1016/j.jconrel.2020.01.028. PubMed PMID: 31962095, and Sun Q, Silva E A, Wang A, Fritton J C, Mooney D J, Schaffler M B, Grossman P M, Rajagopalan S. Sustained release of multiple growth factors from injectable polymeric system as a novel therapeutic approach towards angiogenesis. Pharmaceutical research. 2010; 27(2):264-71. doi: 10.1007/s11095-009-0014-0. PubMed PMID: 19953308; PMCID: 2812420.

The data presented above showed that when comparing mice wounds treated with control versus those treated with metrifonate, a pooled standard deviation of 11.6 and a maximum difference 24.3 was the result which indicates that 7 or 8 mice in each group can enable detection of a difference in wound healing improvement with more with 80% or 90% power and 5% level of significance. Similar results were observed for the other tested parameters. In order to cover possible attrition due to animal loss during the whole experiment, 10 mice can be used in each group. Utilizing 10 mice per group to ascertain that we will be able to capture microvariations within groups or phenotypes of toxicity at the molecular level.

Comparisons between two groups can be performed using the t test for normally distributed data and the Mann-Whitney test for data that are not distributed normally. When more than two groups will be compared the ANOVA and Kruskal-Wallis tests will be used respectively.

As prior animal model for DFU have limitations, at least two animal models are being used to test including the diabetic Yucatan miniature pig model. The skin of pigs is similar to the human skin in many aspects, including thickness of epidermis, collagen composition, hair follicle density, adherence to the underlying structures, epidermis turnover and types of immune cells. Yucatan miniature pigs are being used as they are very similar to Yorkshire pigs that have already been shown to have diabetes-related impaired wound healing that is similar to the human condition but their smaller size of the Yucatab Pig makes them more manageable. It is important to evaluate the mechanisms of action of metrifonate and evaluate whether the obtained results are in concordance with the in silico screen assumptions, confirming the hypothesized mechanisms of action. Furthermore, evaluation of the mechanism of action using bulk, single cell and spatial transcriptomic analysis provides valuable information about the mechanisms of action of metrifonate and identification of differences between mice, pig and human tissue.

A diabetic Yucatan minipig model has been used in recent studies that evaluated the efficacy of new biomaterials for treatment of wounds. The creation of 18 wounds (nine in each side) allows the comparison between various treatments and control treatment, which can include the application of Tegaderm on the wound area (see FIG. 9).

Excisional full-thickness skin wounds of diabetic Yucatan miniature pigs can confirm the efficacy and appropriate doses that to guide human Phase I/II clinical trials. Four pigs (two males and two females) and diabetes are induced using alloxan by the vendor (Sinclair Bio Resources, LLC/562 State Rd. Auxvasse, MO 65231) before their arrival for study. Two weeks after diabetes induction 24 square (15×15 mm) excisional full thickness wounds are created on the paravertebral and thoracic area using an acrylic board (20 by 30 cm) template with a distance of 2.5 cm of unwounded skin between each wound (see FIG. 9). The efficacy of the best two Metrifonate-Hydrogel doses that have been identified in the prior mice studies. In order to scale up the dose for the pig wounds, the size of the wound determine identify specific drug dose amount per cm² of wound area.

At the beginning of the study, for the first two pigs (one male and one female), each wound will be treated with one of the following conditions: A.) Metrifonate-Hydrogel dressing-dose 1; B.) Metrifonate-Hydrogel dressing dose-2; C.) Blank Hydrogel/Alginate+Laponite only; D.) Control, no treatment/Tegaderm only. Treatments can be placed in a way that all four treatments contain wounds with similar characteristics regarding their distribution and anatomical position, including distance from the head and the spine. All wounds can be covered with Tegaderm. Three days following skin wounding and initial treatment, the dressings can be permanently removed and wounds protected with Tegaderm for the reminder of the study (7 additional days) prior to animals being euthanized at 10 days. Using this model, there can be nine wounds in each pig treated with the same experimental conditions. In the last two pigs (one male and one female), allocation and removal of the different experimental dressings can be done the same way as described above, however, wound size can be assessed at 5, 10, 15 and 22 days post-operative treatment, followed by wound biopsy of two individual wounds of each group at each time point. Final wound size assessment and wound harvest can be done at Day 28, when the animals will be sacrificed. Upon sacrifice, all wounds and surrounding skin can be excised and the same tests that are described in connection with the mice study can be performed.

Temporal whole transcriptome (bulk) analysis can be performed with 4 biological replicates in each group, for example. Wounds from the above experiments involving pigs, humanized mice and ex vivo human skin can be analyzed to investigate the effects of the treatment in different doses across the time axis. Systems/Network biology analyses can be employed to identify master regulators and their temporal expression in wound healing with and without the presence of metrifonate. Single cell RNAseq: as described above, an extensive single cell map of human wound healing has been formed, and mouse samples with all available relevant mouse studies have been used for this purpose. To utilize these resources to identify the effects of metrifonate at the cellular level, scRNAseq data is being generated from mouse, pig, and human samples from controls and following metrifonate treatment at 2 doses. Improved time point selection can be performed based on the bulk RNAseq studies. In brief, rapid automated tissue dissociation can be performed from harvested tissue samples using the automated Singulator 100 (S2 Genomics) to minimize batch variations in gene expression resulting from conventional tissue dissociation procedures. Cell viability and number will be evaluated with a Luna-FX7 (Logos Biosystems). An estimated 8,000 cells can be sequenced per sample. Automated 3′ scRNAseq will be performed with the 10× Genomics Connect robotics platform using the vendor's standard protocol with minimal manual steps. The following stop points can be used: following GEM generation to photo document quality, prior to cDNA amplification to QC 1 μl of cDNA on a Fragment Analyzer (FA, Agilent), and library QC using the FA. Only libraries passing QC will be forwarded for sequencing on an Illumina NovaSeq 6000 and any failed samples will be repeated. Single cell data analysis can be performed as previously described. Multiplexed Immunofluorescence: 6-plex antibody panels can be generated for the Akoya PhenoImager whole slide multispectral scanning platform (Vectra Polaris). The panels can be automated on a Leica Bond Rx autostainer to be performed across the study samples with high repeatability and low noise. Akoya inForm software can be utilized to segment the whole slide scans and quantify the markers per region of interest and per cell, providing quantitative data for the temporal and group comparisons.

To analyze the effect of metrifonate on cell-cell interactions during wound healing further methods in addition to multispectral immunofluorescence can be used. To this end, Multiplexed error-robust fluorescence in situ hybridization (MERFISH) can be used enabling quantification of the expression of up to 500 genes in a tissue section at single cell and single molecule resolution. The MERFISH workflow builds upon the highly standardized and optimized implementation on the MERSCOPE instrument (Vizgen, MA). This production-grade pipeline addresses many major hurdles of the classic MERFISH assay, including stitching, registration, and segmentation of image data. The commercial MERSCOPE instrument enables analysis of a sectioned tissue block that is placed on the MERSCOPE slide (Vizgen) and permeabilized overnight, following 5 mL 70% ethanol addition. For autofluorescence quenching, the slide is placed in the MERSCOPE Photobleacher (Vizgen) for 3h. Formamide wash buffer is added and incubated at 37° C. for 30 min. 50 μL MERSCOPE Gene Panel Mix is added to the section and incubated at 37° C. for 40h. 50 μL Gel Slick Solution is placed onto the Gel Coverslip and 5 ul of Gel Embedding Solution is added and following 1′, 50 μL of the Gel Embedding Solution is placed on the tissue section and incubated at room temperature for 1.5 h. The Vizgen Clearing Premix and Proteinase K are used to clear the tissue, while keeping RNA/DNA intact, by following the human liver-specific protocol. Samples are incubated for 24 h or until the tissue section becomes transparent, and then are incubated with DAPI and PolyT Staining Reagent for 15′. Slides are placed into the MERSCOPE and scanned. Up to 1 cm² will be analyzed using a probe set capturing up to 500 targets. Note that 118 targets (see FIG. 9) have been selected by reanalyzing the internal single cell data for wound healing in human and mouse. Additional targets can be incorporated into the assay with the bulk and single cell studies.

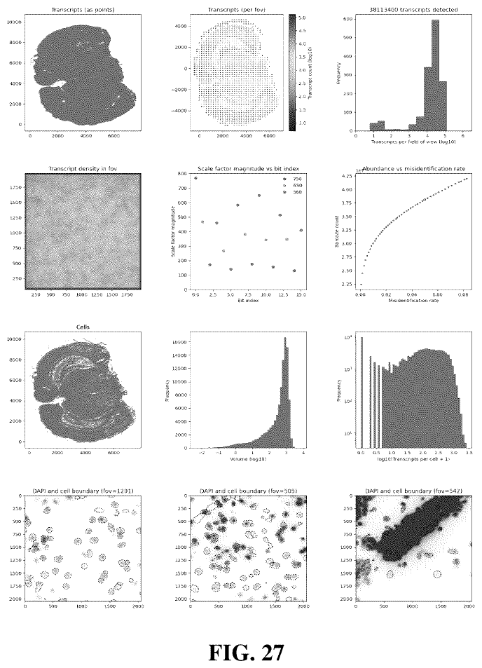

MERSCOPE files are converted to Zarr-AnnData and OME-TIFF format. Extensive QC metrics are extracted (FIG. 27), including the number of cells and RNA molecules detected, sample dimensions, RNA molecule density, and statistics per probe to enable identification of low-performing probes. The multiplexed immunofluorescence assays from serial sections can be overlayed using non-rigid slide registration. MERSCOPE and overlayed multiplexed IF datasets can be further explored with Giotto (see Dries et al., “Giotto: a toolbox for integrative analysis and visualization of spatial expression data”, Genome Biology: 2021; 22(1):78.doi:10.1186/s13059-021-02286-2, the entire contents thereof being incorporated herein by reference) to perform differential expression analyses, cell-cell colocalization and communication, as well as to identify spatially variable genes. Spatially distinct regions can be detected and scRNAseq data can be integrated and registered on the spatial assays using Tangram. The MERSCOPE datasets will be utilized to identify the effects of metrifonate on the tissue microenvironment in situ and to maximize our ability to interrogate tissue and to investigate findings from the bulk, proteomic, and single cell studies.

Experience has shown that the effect of size in pig studies was similar, if not better, to mice studies. The same can apply for this study and, as a result, nine wounds per treatment can be sufficient to provide more than 80% power at 5% level of significance. Across all modalities (single cell, spatial), 4+ biological replicates can be utilized per group/timepoint enabling us to achieve high granularity and power in all comparisons. There can be more than 99% power to detect rare cellular populations (less than 1% of cells) with an adequate number of cells per cluster (>100) for at least 10 rare cell populations per group.

Comparisons between groups can be performed using the ANOVA test for normally distributed data and the Kruskal-Wallis test for data that are not distributed normally. The Fit General Linear Model can be employed for repeated measurement analysis. RNA-seq data can be processed with data being quality-checked using FastQC⁵⁵ and reads can be aligned to reference genome STAR and a 2-pass approach and quantifying reads with featureCounts(72). Differential gene expression analysis can be performed using DESeq2(55), while ClusterProfiler (v3.12.0)(73) can be utilized for downstream functional investigations.

Other complementary assays, including single nucleus sequencing (in lieu of single cell sequencing) as well as different additional spatial tissue profiling platforms such as 10× Genomics Visium, Akoya CODEX, NanoString GeoMx DSP, and others, which can be utilized to substitute or complement the above assays.

Confirmation of the obtained mouse and pig results in ex vivo human skin and the in vivo humanized skin graft mouse model demonstrates that metrifonate is suitable for DFU treatment.

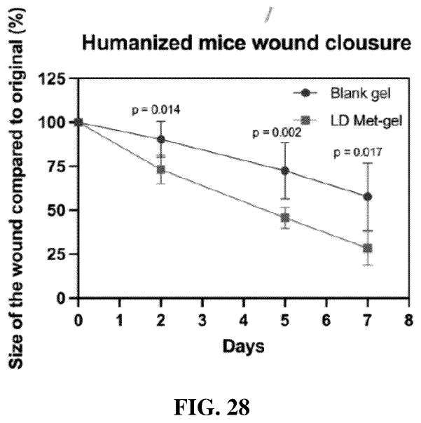

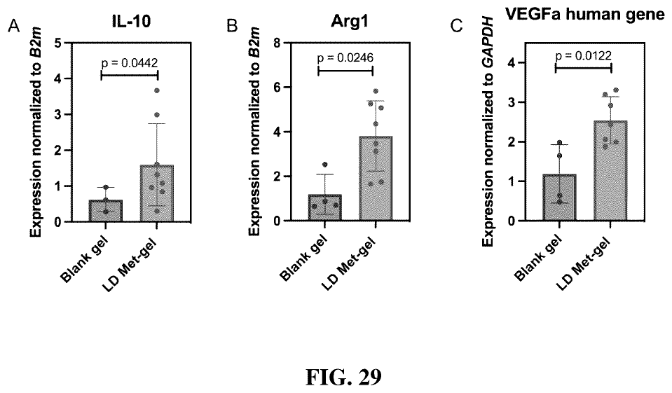

Employing the techniques described below, circular biopsy punch 3-mm full-thickness wounds on the transplanted human skin of diabetic humanized mice are used and treated either with blank hydrogel or hydrogel releasing the low dose of metrifonate (FIG. 11A,). Metrifonate demonstrated improved wound healing, to a degree that was similar, if not better, to the one that was observed in db/db mice (FIG. 28). RT-qPCR gene expression analysis upon sacrifice of the animals showed that metrifonate application on the wound increased the expression of 1110 (FIG. 29A), Arg (FIG. 29B), and human gene VEGFA (FIG. 29C). These genes are well known to improve impaired diabetic wound healing, see Berry-Kilgour et al., Advancements in the Delivery of Growth Factors and Cytokines for the Treatment of Cutaneous Wound Indications; Adv Wound Care (New Rochelle) 2021; 10(11):596-622. Epub 20201125. doi:10/1089?wound.2020.1183. PubMed PMID:33086946: PMCID: PMC8392095.

Human skin transplantation on immunodeficient mice, which support engraftment, are a valuable study model for investigating mechanisms involved in wound healing. It has been demonstrated in various studies that, after injury, the regeneration of human skin transplanted onto nude mice mimics the process observed in injured patient skin; thus making the establishment of a humanized diabetic wound healing model a valuable resource for studying the wound healing process in vivo. Human skin grafted onto the nude mouse has previously been shown to be able to regenerate after an excisional wound is made through the entire thickness of the graft.

To generate a humanized skin graft mice model, Foxn1nu nude male mice of 6 weeks old can be used as the recipient animals. These mice are devoid of T cells and suffer from a lack of cell-mediated immunity making them a model of choice for xenograft studies. Mice dorsal skin will be excised with an area where a graft of an equal size of full-thickness or split-thickness human skin will be sutured (FIG. 11A). Human skin is obtained from IRB-approved discarded human skin from plastic surgeries. Grafted mice are left to heal and accommodate the new tissue for 5 weeks, where the graft fully matures. Diabetes is induced in stable human skin engrafted mice 5 weeks post-grafting via intraperitoneal injections with streptozotocin (STZ). Mice sustain a hyperglycemic state for 7 weeks prior to wounding experiments. Following the sustained diabetic state period, wounds are inflicted on the transplanted human skin with a 3 mm punch biopsy, treatment options include the best two Metrifonate-Hydrogel dressings, previously determined by the mice and pig studies for the preferred two doses. These dressings are applied to each individual wound, as well as a blank hydrogel formulation. As bandages can provide sustained release for at least three days, and changes can be initially perform every three days while the potential of weekly changes will be explored with the chosen two best doses. At days 3, 5 and 10 post-wounding and dressing treatment, mice are euthanized and wound tissue can be harvested for histological and molecular analysis. Wound healing can be monitored using standardized photography daily during the course of 3, 5 and 10 days. Wound area can be presented as % of original wound. Histological and immunohistochemistry studies being performed as described for the mice studies.

To further examine the effects of Metrifonate-Hydrogel on human wound healing, wounds on panniculectomy derived discarded skin can be used. Ex vivo human skin culture wound healing study skin specimens can be obtained from BioIVT (Westbury, NY) and derived from abdominoplasty procedures. De-identified samples are provided with removed subcutaneous adipose tissue in sterile PBS at 4° C. on the day of surgery and are immediately processed. A 6 mm biopsy punch will be used to gently punch the skin's epidermis partially penetrating into the dermis to create a wound. The best performing Metrifonate-Hydrogel and a Blank Hydrogel formulation can be topically applied and the skin specimens will be placed dermis side down onto the culturing dishes and incubated at 37° C., 5% CO2. Healing can be monitored for four days using the techniques as in a recent study that ex vivo involved human skin. Similar transcriptomic and proteomic techniques to those described above can be employed to evaluate in vivo and ex vivo effects of metrifonate in human tissue.

The main endpoint will be the same as with the mice studies described in Aim 1, namely reduction in the wound size and the secondary endpoints will include re-epithelization, granulation and angiogenesis. We expect that for the chosen two doses the results will be similar or better to the ones that will be observed in mice and are presented. In addition, we expect that transcriptomic analysis will show that metrifonate application will have a very similar profile to that obtained in DFU patients who healed the ulcers.

The data presented above showed that when comparing humanized mice wounds treated with control versus those treated with metrifonate, we had a pooled standard deviation of 29 and a difference 30, which indicates that 16 wounds in each group will allow us to detect a difference in wound healing improvement with more with 80% power and 5% level of significance.

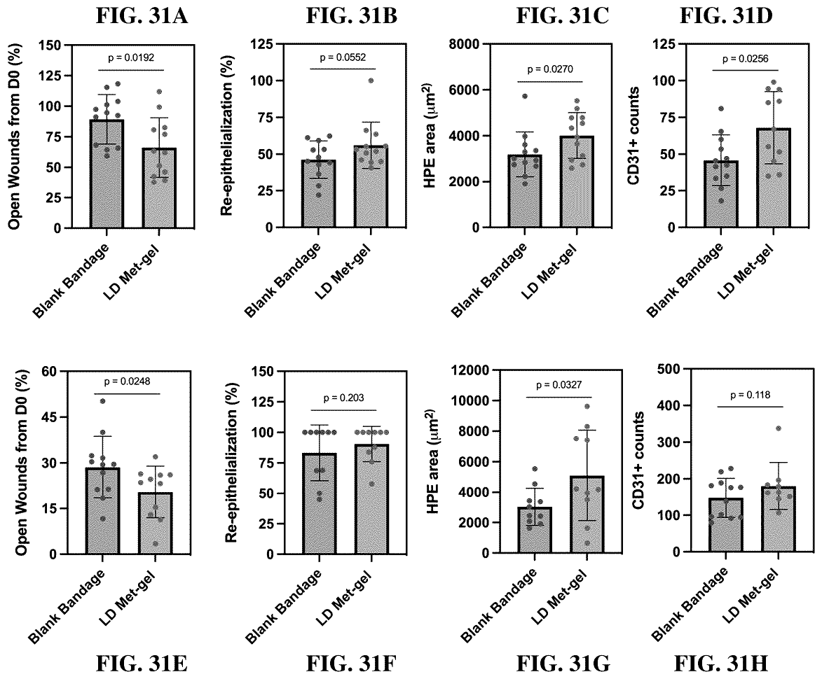

The results above showed the high wound healing shown for low dose levels of metrifonate gel (LD-Met gel) with a day 3 removal treatment on db/db mouse model, a well-established model of impaired diabetic wound healing. These results indicated that metrifonate exerts its therapeutic effects in the early inflammatory stage of wound healing rather than the subsequent proliferative or remodeling phases. To further investigate the mechanism of action, an earlier sacrifice time point, Day 5, was used to provide molecular insights and cell functions involved in the healing process. The wound closure rate (compared to the size of wound on day 0), percentage of re-epithelialization, blood vessel (CD31+ structures) counts, and hyperproliferative epidermis (HPE) area on both day 5 and day 10 were analyzed for three treatment groups: blank gel (blk gel) and LD-Met gel (FIG. 31). Compared to Blk-Gel treated wounds on day 5, the LD Met-Gel significantly accelerated wound healing with a higher re-epithelialization rate (FIG. 31B) and vessel density (FIG. 31D). On day 10, except wound closure rate (FIG. 31E) and HPE area (FIG. 31G), no significance was observed for re-epithelization, and CD31 counts (FIGS. 31F & H). These results indicated that application of low doses of metrifonate during early wound healing, coinciding with the inflammatory phase, improve impaired diabetic wound healing.

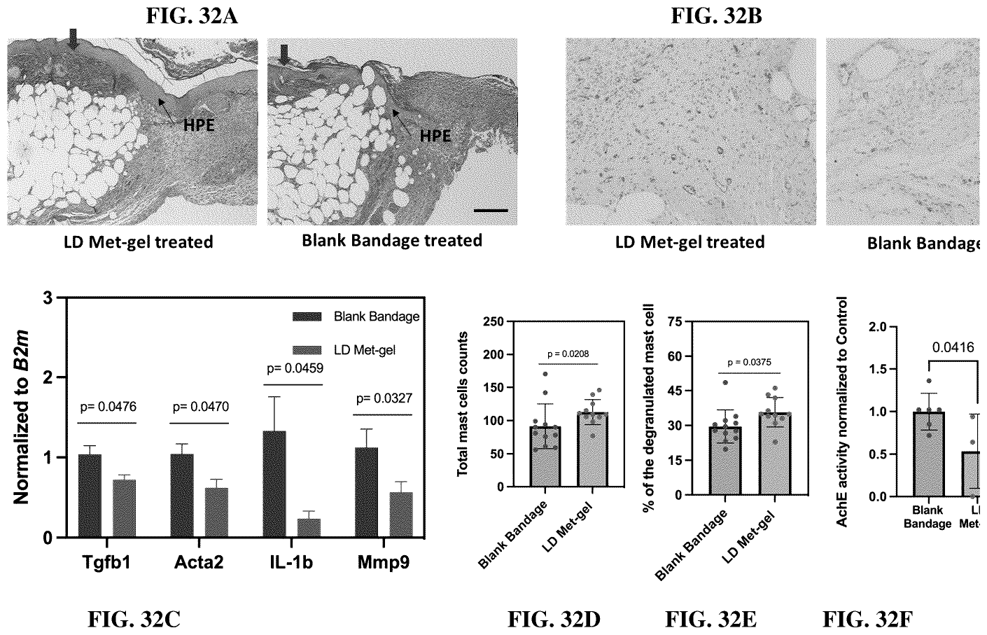

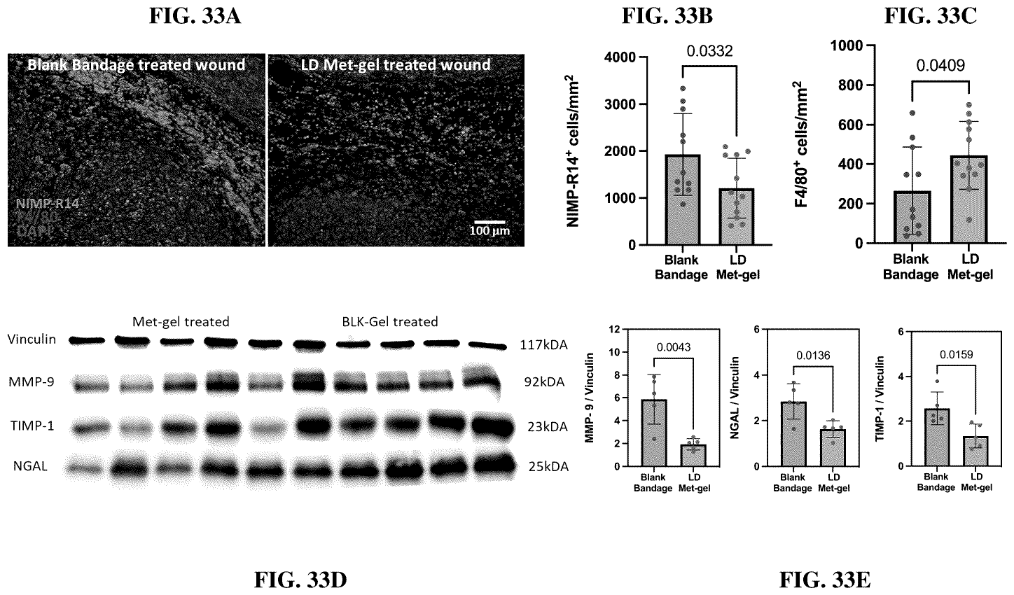

When comparing D5 wounds treated with LD Met-gel removed at D3 with blank gel treated wounds, multiple healing metrics were positively regulated including increased re-epithelialization (FIG. 32A) and angiogenesis (FIG. 32B), as well as reduced fibrosis (Tgfb1, Acta2) and inflammation (Il1b, Mmp9) markers gene expression (FIG. 32C). Intriguingly, metrifonate led to enrichment of both total and degranulated mast cells, as discerned by toluidine blue staining (FIG. 32D). We have previously identified these granulocytes as integral mediators of diabetic wound healing. We also validated that metrifonate treatment inhibits Acetylcholinesterase (AchE) activity by measuring the levels of the enzyme on Day 5 mouse wounds (FIG. 32F), a time point that is 2 days after bandage removal. Collectively, these findings suggest that metrifonate elicits pleiotropic pro-reparative effects to impact important wound healing processes. Further, we found that metrifonate showed a significant effect in modulating inflammatory response by staining for two major cell types that are involved in the early inflammatory stage, neutrophil and macrophages (FIGS. 33A-33E). Metrifonate bandage application decreases the levels of neutrophil marker NIMP-R14+ cells (FIG. 33A green, 33B) but increases the number of macrophage marker F4/80+ cells (FIG. 33A red, 33C). This corroborates the findings indicating that metrifonate promotes neutrophil clearance and facilitates monocyte to macrophage differentiation. The protein expression of both MMP-9 and NGAL (markers for neutrophils) were down regulated by LD-Met gel (FIG. 33D&E).

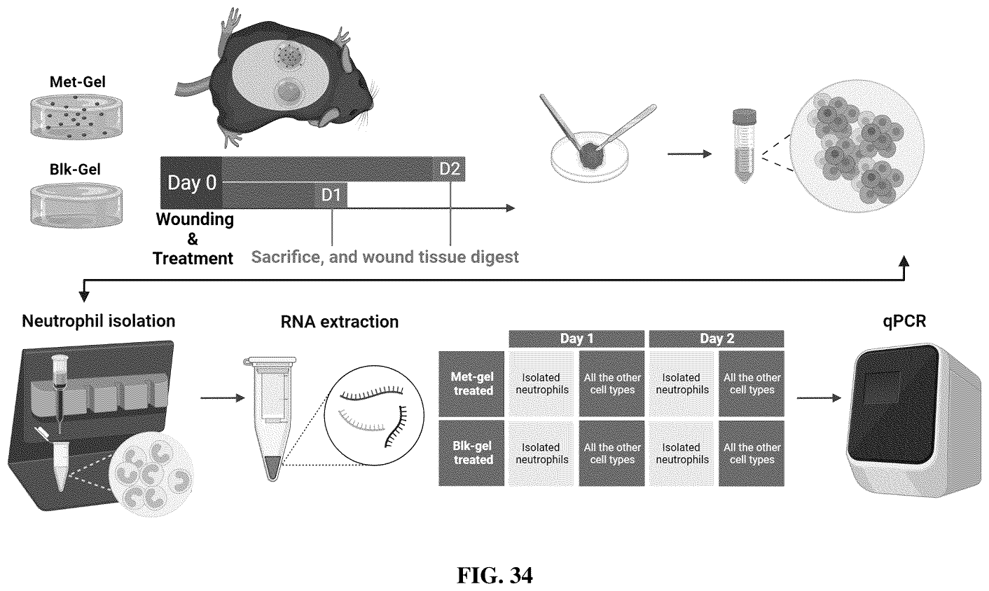

To further explore the dominating cell types that are responsive to metrifonate and the mechanisms of action, an in vivo study was conducted using db/db mouse model with a day 1 and day 2 sacrifice time points (FIG. 34). Blank gel served as control, and LD-Met gel was the selected treatment. Previous studies have shown the vast majority of skin cells express muscarinic and/or nicotinic receptors and could be impacted by fluctuations in Acetylcholine (Ach) levels. In this in vivo study, the primary objective was to elucidate the metrifonate's mechanism of action, particularly in terms of activation of muscarinic and/or nicotinic receptors. We hypothesized that the metrifonate predominantly impacts neutrophils, making them a major target cell type, while not entirely eliminating the influence on other cell types. To this end, our study was divide into two groups, and wounds from a treated group and a control group were collected on day 1 and day 2 of treatment respectively, and were digested to generate highly viable single cell suspension.

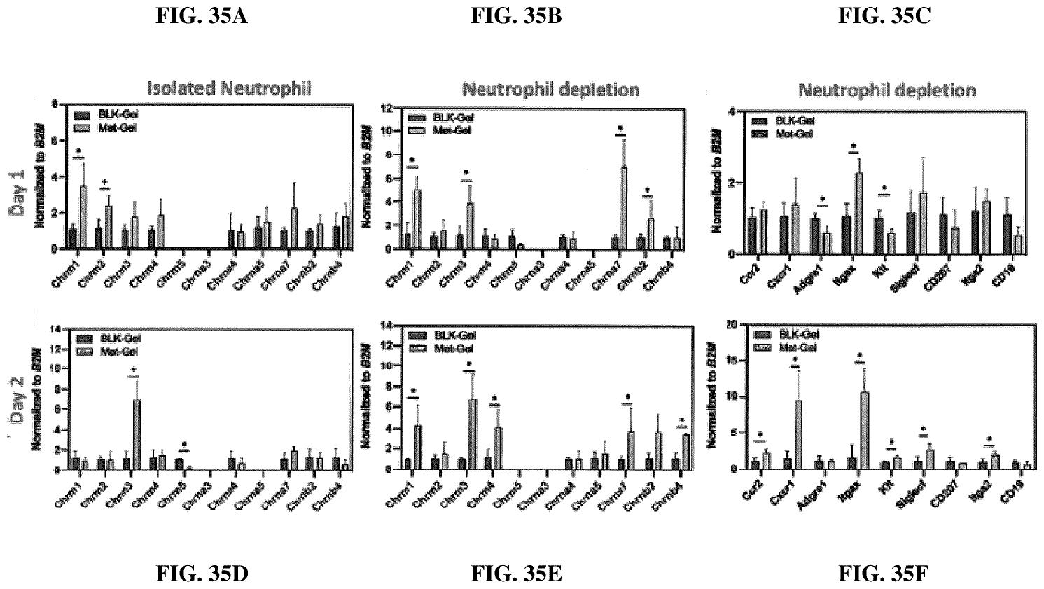

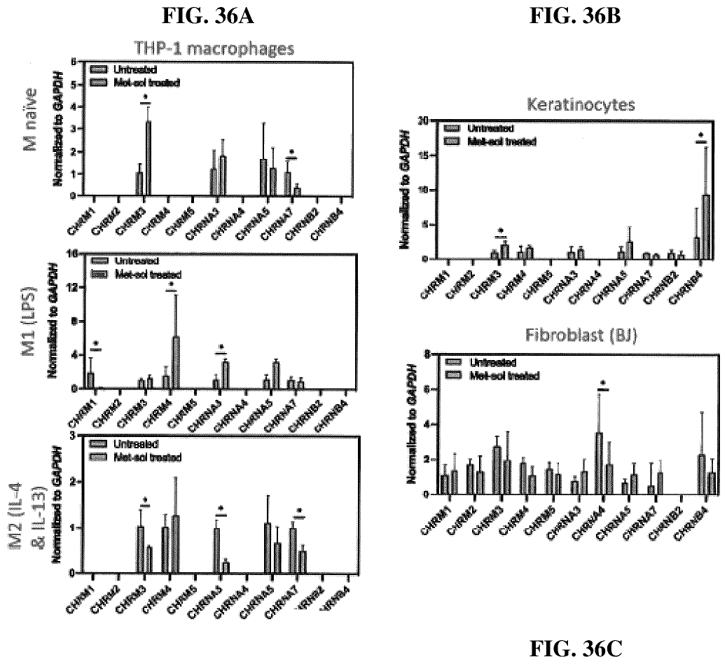

Then, we utilized magnetic sorting techniques to separate the cells into neutrophil-rich and non-neutrophil fractions and extracted RNA from both (FIG. 34). We then performed gene expression analyses of isolated neutrophils and the rest of the wound cells on day 1 and day 2, using a panel of nicotinic and muscarinic receptors (FIG. 35A-B, 35D-E). The gene expression of Chrm1 and Chrm2 for isolated neutrophil from the wounds treated by LD-Met gel on day 1 were upregulated (FIG. 35A), whereas only Chrm3 expression showed a 7-fold change in isolated neutrophil collected from LD-Met gel treated wounds compared to neutrophils in blk gel treated wounds on day 2 (FIG. 35D). Intriguingly, Chrm1, Chrm3, Chrna7 were markedly activated in the rest of the cell types by LD-Met gel on day 1 compared to blk gel, and remained continually activated by LD-Met gel through day 2 (FIG. 35E). To further explore the specific cell types that may involve in this process, RT-qPCR gene expression analysis was carried out for the rest of the cell collections from the wounds. Here, specific primers representing canonical markers for different cell types were selected for both day 1 and day 2 (FIGS. 35C & 35F). The LD-Met gel facilitated dendritic cell recruitment on both day 1 and day 2. Of note, monocytes, mast cells, eosinophilic cells, and natural killers were recruited to the wound site by LD-Met gel treatment (FIGS. 35C & 35F). The nicotinic and muscarinic receptors gene expressions were further studied in human primary cell lines with important roles in the wound healing process, including THP-1 monocyte derived macrophages (M0 naïve macrophages, LPS activated M1 macrophages, and IL-4 & IL-13 activated M2 macrophages), keratinocytes, and fibroblasts (FIG. 36A-36C). RT-qPCR was performed after these cells were treated with or without metrifonate for 48 hours, using the same panel of nicotinic, muscarinic receptors utilized in above in vivo mouse study. The results indicated divergent responses according to the cells' polarization with MO cells displaying activation of CHRM3 receptor and M1 cells exhibiting activation of CHRM4 (FIG. 36A), while CHRNA3 receptor was also more markedly activated in these cells (FIG. 36A). Surprisingly, metrifonate down-regulated CHRM3, CHRNA3, and CHRNA7 expression in M2 cells (FIG. 36A). Keratinocytes responded to metrifonate through activation of CHRM3 and CHRNB4 receptors (FIG. 36B), while fibroblasts predominantly exhibited activation of CHRNA4 (FIG. 36C).

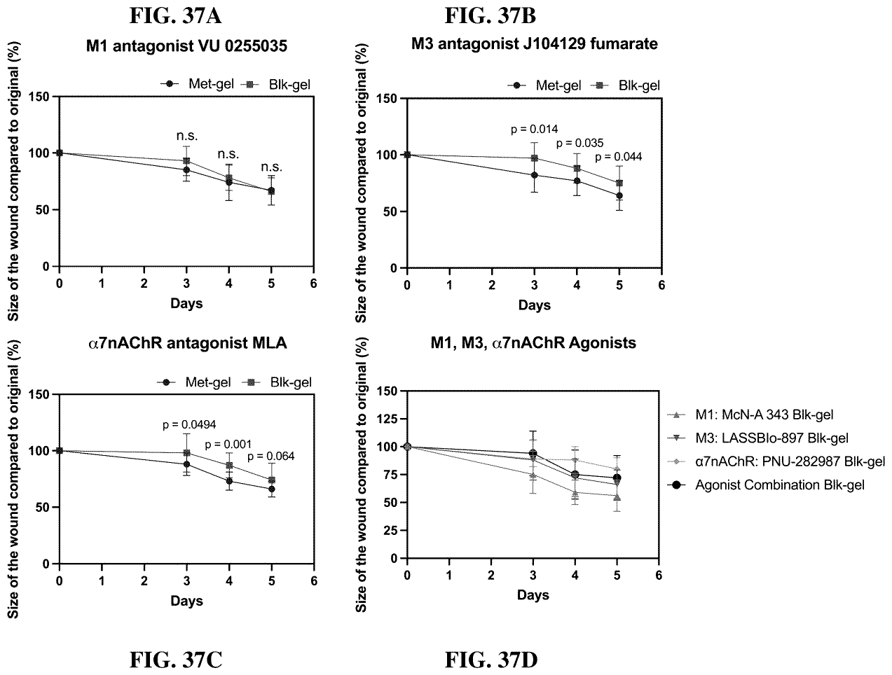

The above in vivo, and in vitro findings reveal important mechanistic insights to provide more accurately decipher metrifonate mode of action. To demonstrate that metrifonate acts through Chrm1 (M1), Chrm3 (M3) muscarinic receptors or α7nAChR nicotinic receptor, another in vivo study was conducted using db/db mouse model with day 5 sacrifice as illustrated in FIGS. 37A-37E. Highly selective agonists or antagonists were chosen for M1, M3, and α7nAChR receptors and topically applied (2 mg/kg) to the wound area daily. Notably, with M1 antagonist (VU0255035) applied, LD-Met gel treated wounds performed comparably to Blk gel treated wounds (FIG. 37A), whereas M3 (J104129) and α7nAChR (Methyllycaconitine citrate) antagonists had no impact on wound closure rate to both treatment groups when compared to LD-Met gel and blk gel treated wounds with no application of any antagonist. (FIGS. 37B & 37C). These findings indicate that metrifonate exerts its therapeutic effect through activation of Chrm1 receptor. To further validate the function of Chrm1 receptor, we employed Chrm1 (McN-A 343), Chrm3 (LASSBIo0897), and α7nAChR (PNU-282987) agonists, molecules that can bind and functionally activate the target receptor, separately to the wound area daily with blk-gel treatment to confirm if the agonist(s) are capable of mimicking the function of metrifonate (FIG. 37D). Of note, solely the M1 agonist promoted the wound healing, and outperformed M3 and α7nAChR agonists. Taken together, these experiments suggest that metrifonate enhances wound healing mainly through the activation of Chrm1 receptor.

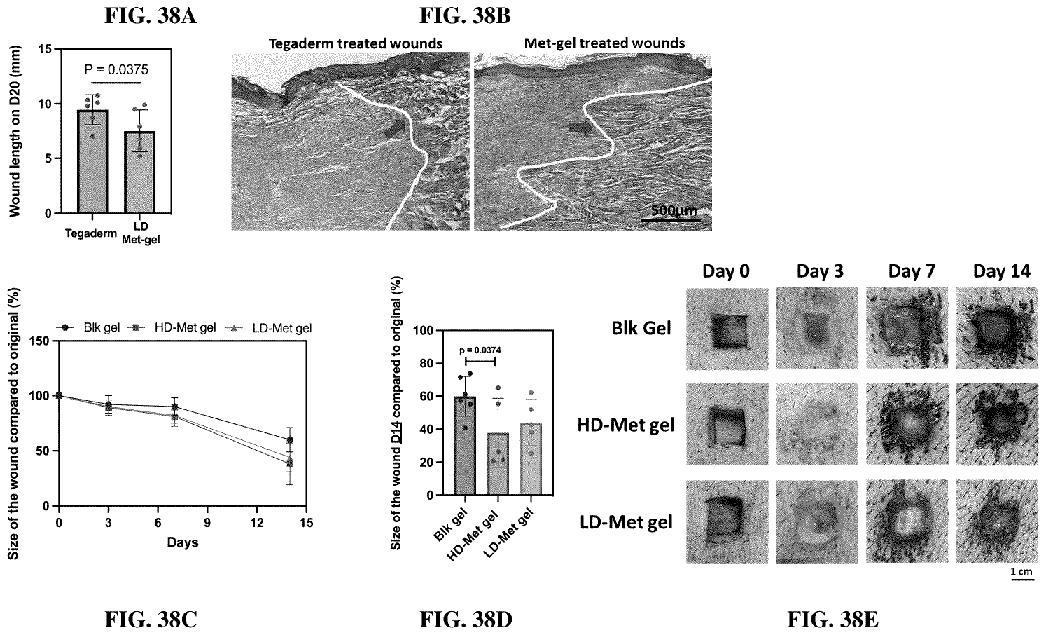

As noted previously, as there is no satisfactory animal model for DFU, it is currently recommended that two animal models be tested, diabetic Yucatan miniature pig model were chosen to confirm the proof of efficacy. Porcine skin is similar to human skin in many aspects, including thickness of epidermis, collagen composition, hair follicle density, adherence to the underlying structures, epidermis turnover and types of immune cells. Yucatan miniature pigs closely resemble Yorkshire pigs that have been shown to have diabetes-related impaired wound healing that is similar to the human condition but their smaller size makes them more manageable. Our experience with the diabetic Yucatan minipig model in recent studies assessed the efficacy of new biomaterials. The creation of 24 wounds (twelve in each side) allows the comparison between various treatments and a control treatment, which is usually the application of Tegaderm on the wound. Experiments performed seeking to explore feasibility and establish the safety of the metrifonate eluting dressings in a larger pre-clinical model. To this end the biomaterials were placed on acute porcine wounds over 20 days and found that metrifonate did not induce any adverse effects and promoted better healing as evidenced by shorter wound lengths in histological assessment (FIGS. 38A & 38B). In addition, diabetic Yucatan miniature pigs were used to confirm the efficacy and appropriate doses to guide human clinical trials. Four weeks after diabetes induction, 24 square (15×15 mm) excisional full thickness wounds were created on the paravertebral and thoracic area with a distance of 2.5 cm of unwounded skin between each wound. To scale up the dose for the pig wounds, the efficacy of the two Metrifonate-Hydrogel doses, High dose-Met gel (HD met-gel) and Low dose (LD-Met gel) were evaluated with blk gel serving as experimental control. The wound size was assessed at Days 3, 7, and 14 post-injury and all treatments were removed on Day 3 (FIG. 38C-38E). Both HD-Met gel and LD-Met gel achieved higher healing rate when compared to the blk gel. Thus, the application of metrifonate has been demonstrated to be effective in advancing wound healing during the inflammatory phase of wound healing relative to the rate and extent of healing in treatments without metrifonate.

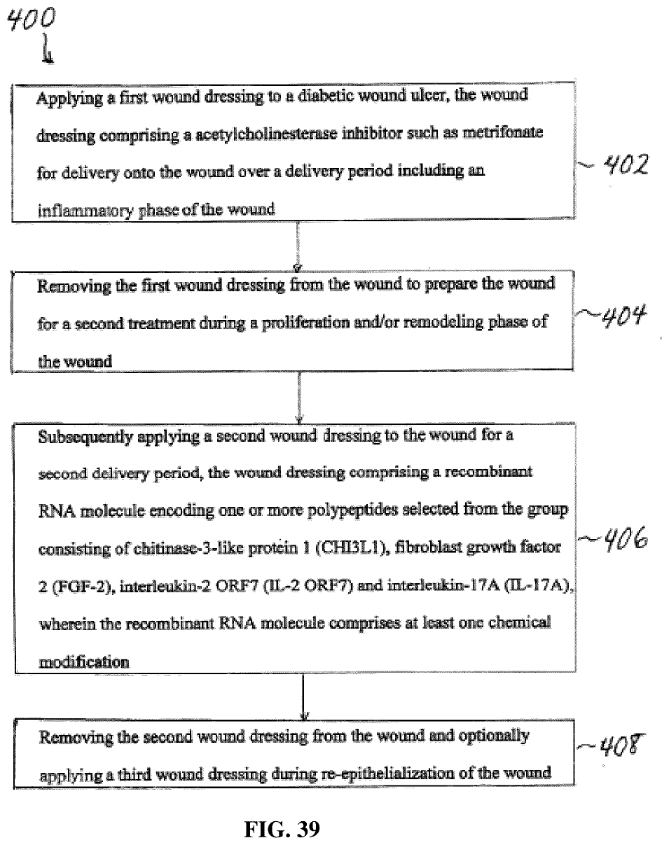

In a further method for the treatment ulcerative wounds using an acetylcholinesterase inhibitor, a first therapeutic dose of the acetylcholinesterase inhibitor is delivered to the wound during the inflammatory phase of wound healing and a therapeutic dose of a recombinant RNA molecule as described herein can then be applied to the wound after the inflammatory phase to improve wound healing during the proliferative and/or regenerative phase of wound healing. In exemplary embodiments, the method 400 illustrated in FIG. 39 can be used wherein a first dressing can be applied 402 to the wound for the delivery of an acetylcholinesterase inhibitor such as metrifonate to improve healing during the inflammatory phase and subsequently applying a second further wound dressing to the wound during the proliferative and/or regenerative phase. After removal of the first dressing 404, the second wound dressing can be used to deliver 406 a therapeutic dose of a recombinant RNA molecule that encodes one or more polypeptides selected from the group consisting of chitinase-3-like protein 1 (CHI3L1), fibroblast growth factor 2 (FGF-2), interleukin-2 ORF7 (IL-2 ORF7) and interleukin-17A (IL-17A). The second dressing can be placed onto the wound after the removal of the first dressing. A third dressing can optionally be placed onto the wound 408 after the removal of the second dressing until re-epithelialization of the wound. As the polypeptides interleukin-2, interleukin-17A and fibroblast growth factor 2 have been shown to improve healing during later stages of the wound healing process, the second wound dressing can include one or more of these polypeptides for delivery onto the wound during the second delivery period.

The recent successful completion of studies that employed both humanized mice and ex vivo human skin provides evidence of efficacy, however a switch in the type of the immunocompromised mouse and/or use human skin specimens for other body areas, including skin from amputated legs, can be used to model wound therapies as described herein.

### Example 19. Evaluation of Low Dose-Metrifonate Gel in Diabetic Wound Healing in a db/db Mouse Model

### Rationale

Previous studies showed that the highest wound healing potential was observed for Low Dose-Metrifonate gel (LD-Met gel) with a day 3 removal treatment on db/db mouse model, a well-established model of impaired diabetic wound healing. We thus hypothesized that metrifonate exerts its therapeutic effects in the early inflammatory stage of wound healing rather than the subsequent proliferative or remodeling phases.

### Methods

To delve into the mechanism of action of metrifonate, an earlier sacrifice time point at Day 5 was introduced, so as to capture molecular insights and study cell functions involved in the healing process.

### Results

Wound closure rates were quantified on Days 5 and 10 and compared to Day 0 for two treatment groups, namely, blank gel and LD-Met gel. The data, expressed as percentage of open wound compared to Day 0, showed significantly accelerated wound healing for the LD-Met gel group compared to blank gel on Day 5 (FIG. 24A) and Day 10 (FIG. 24E). Consistent with this result, higher re-epithelialization rates (FIG. 24B), as well as higher Hyperproliferative Epidermis (HPE) areas (FIG. 24C), and higher numbers of CD31+ structures (FIG. 1D) were observed on Day 5. While wound closure rates and HPE area remained significantly higher for LD-Met gel as compared to blank gel on Day 10 (FIGS. 24E and 24G, respectively), a comparison of re-epithelization rates and CD31+ counts revealed no significant differences between both groups (FIGS. 24F and 24H, respectively). These results indicated that application of low doses of metrifonate during early wound healing, coinciding with the inflammatory phase, improved impaired diabetic wound healing.

### Example 20. Impact of LD Met-Gel on Processes Associated with Wound Healing

### Methods

Wounds were treated with an LD Met-gel that was removed at Day 3. Multiple metrics of healing for these wounds were measured at Day 5, and compared with blank gel treated wounds.

### Results

Imaging of Day 5 wounds with Mason's Trichrome Stain (MTS) showed increased re-epithelialization (representative images in FIG. 25A), while immunohistochemical (IHC) staining for CD31+ revealed improved angiogenesis (representative images in FIG. 2B) for LD Met-gel group compared to blank gel. A qRT-PCR analysis of gene expression of markers of fibrosis (Tgfb1 and Acta2) and inflammation (Il1b, Mmp9) revealed reduced fibrosis and inflammation in the LD Met-gel treated group (FIG. 25B). Intriguingly, use of metrifonate led to enrichment of both total and degranulated mast cells, as discerned by toluidine blue staining (FIGS. 25D and 25E, respectively). These granulocytes have previously been identified as integral mediators of diabetic wound healing. We also validated that metrifonate treatment inhibits the activity of Acetylcholinesterase (AchE) by measuring the levels of the enzyme on Day 5 in mouse wounds lysates (FIG. 25F), a time point that is 2 days after bandage removal. Collectively, these findings suggest that metrifonate elicits pleiotropic pro-reparative effects to affect important wound healing processes.

### Example 21. Effect of Metrifonate on the Inflammatory Response

### Methods

The effect of metrifonate in modulating inflammatory response was assessed by the presence of neutrophils and macrophages that are involved in the early inflammatory stage, and by analysis of protein expression in metrifonate treated db/db mice wounds compared to blank gel in Day 5 diabetic mouse wounds.

### Results

Immunofluorescent imaging of wounds revealed that application of a metrifonate bandage reduced the number of NIMP-R14 positive cells (a neutrophil marker), but increased the number of F4/80 positive cells (a macrophage marker). Representative images are shown in FIG. 26A. These results are quantified in the plots shown in FIGS. 26B and 26C. Next, protein expression in LD Met-gel treated mice wounds was analyzed, and compared to blank gel treated wounds. A Western blot analysis of neutrophil markers MMP-9, NGAL and TIMP-1 showed that their expression was down regulated in LD-Met gels (FIGS. 26D and 26E). These results indicated that metrifonate promotes neutrophil clearance and facilitates monocyte to macrophage differentiation.

### Example 22. Isolation of Neutrophils from Gel-Treated Wounds in db/db Mouse Model

The results thus far suggested that metrifonate's predominant effects were directed at target neutrophils, while still affecting other cell types. To test which cell types respond to metrofinate, we conducted in vivo studies using db/db mouse model with sacrifice time points at day 1 and day 2. The study was divided into two groups, with wounds from both LD-Met gel treated group and control group being collected on days 1 and 2. The samples were subsequently digested to generate highly viable single cell suspensions, and separated into neutrophil-rich and non-neutrophil fractions, through magnetic sorting techniques. RNA was extracted from both fractions. A schematic of the isolation procedure is shown in FIG. 27.

### Example 23. Gene Expression Analyses of Neutrophils and Neutrophil-Depleted Cell Fractions

### Methods

Gene expression patterns of isolated neutrophils and the neutrophil-depleted fractions on days 1 and 2 were analyzed through RT-qPCR experiments. A panel of nicotinic and muscarinic receptors were analyzed, including Cholinergic receptor -muscarinic (Chrm)1, Chrm2, Chrm3, Chrm4, Chrm5, Cholinergic receptor -nicotinic, alpha polypeptide (Chrna)3, Chrna4, Chrna5, Chrna7, Cholinergic receptor -nicotinic, beta polypeptide (Chrnb) 2, and Chrnb4. To identify cell types present in the neutrophil-depleted fractions, specific primers representing canonical markers for different cell types were selected.

### Results

The expression of Chrm1 and Chrm2 genes in neutrophils isolated from the wounds treated by LD-Met gel were found to be upregulated on day 1 (FIG. 28A), whereas on day 2, only Chrm3 expression showed a 7-fold change in isolated neutrophils collected from LD-Met gel treated wounds compared to neutrophils in blank gels (FIG. 28D). Interestingly, in other cell types, Chrm1, Chrm3 and Chrna7 showed markedly higher expression in LD-Met gels on day 1 compared to blank gel, and continued to remain higher on day 2 (FIG. 28E). RT-qPCR experiments that were performed to identify cell types in the neutrophil-depleted fraction revealed that LD-Met gels facilitated dendritic cells recruitment on both day 1 and day 2. Notably, monocytes, mast cells, eosinophilic cells, and natural killers were also recruited to the wound site by LD-Met gel treatment (FIGS. 28C & F).

### Example 24. Gene Expression Analysis of Metrifonate-Treated Human Primary Cell Lines

### Methods

RT-qPCR was used to analyze the expression of nicotinic and muscarinic receptor genes in human primary cell lines that play important roles in wound healing process, such as THP-1 monocyte derived macrophages (M0 naïve macrophages, LPS activated M1 macrophages, and IL-4 & IL-13 activated M2 macrophages), keratinocytes, and fibroblasts. Cells were treated with or without metrifonate for 48 hours, and RT-qPCR experiments were performed with the same panel of nicotinic and muscarinic receptors as used in the in vivo mouse study.

### Results

In response to metrifonate treatment, gene expression patterns varied according to the polarization of the cells, with MO cells displaying up-regulation of CHRM3 receptor, and M1 cells exhibiting higher expression of CHRM4 and CHRNA3 receptors (FIG. 29A). Surprisingly, metrifonate down-regulated CHRM3, CHRNA3, and CHRNA7 expression in M2 cells (FIG. 29A). Keratinocytes responded to metrifonate treatment through up-regulation of CHRM3 and CHRNB4 receptors (FIG. 29B), while fibroblasts predominantly exhibited increased expression of CHRNA4 (FIG. 29C). Taken, together, the gene expression results indicated that metrifonate acts via Chrm1 (M1), Chrm3 (M3) muscarinic receptors or α7nAChR nicotinic receptor.

### Example 25. Validation of Target Receptors of Metrifonate in db/db Mouse Model

### Methods

To confirm the receptors that mediate metrifonate activity in wounds, an in vivo study was performed in the db/db mouse model with sacrifice on day 5. Highly selective agonists or antagonists of M1, M3 and α7nAChR receptors were chosen and topically applied at a dosage of 2 mg/kg to the wound area daily. Wound size was measured on days 3, 4, and 5 post-injury, compared to day 0 for two groups, namely, LD-Met gel treated and blank gel treated wounds.

### Results

LD-Met gel treated wounds performed comparably to blank gel treated wounds in the presence of an M1 antagonist, VU0255035 (FIG. 30A), whereas the presence of M3 (J104129) and α7nAChR (Methyllycaconitine citrate) antagonists had no impact on wound closure rates in LD-Met gel and blank gel treated wounds (FIGS. 30B & 30C). These results indicated that metrifonate exerts its therapeutic effect through activation of Chrm1 receptor. To further validate the role of Chrm1 receptor, we tested Chrm1 (McN-A 343), Chrm3 (LASSBIo0897), and α7nAChR (PNU-282987) agonists, i.e., molecules that can bind and functionally activate the target receptor. These agonists were applied separately to the wound area daily, and their effects were compared to blank gel treatment to confirm if they were capable of mimicking metrifonate activity on wound healing (FIG. 30D). Notably, only the M1 agonist accelerated wound healing under these conditions, and outperformed M3 and α7nAChR agonists. Taken together, these results suggest that metrifonate enhances wound healing primarily through the activation of Chrm1 receptor.

### Example 26. Evaluation of Metrifonate Treatment on Wound Healing in Diabetic Porcine Model

### Rationale

It is currently recommended that two animal models be tested for DFU studies, and we chose diabetic Yucatan miniature pig model to confirm proof of efficacy of metrifonate treatment. Porcine skin is similar to human skin in many aspects, including thickness of epidermis, collagen composition, hair follicle density, adherence to the underlying structures, epidermis turnover and types of immune cells. Yucatan miniature pigs were chosen since they closely resemble Yorkshire pigs that are shown to have diabetes-related impaired wound healing similar to the human condition. The smaller size of Yucatan miniature pigs makes them more manageable.

### Method

Initial experiments were performed to establish the safety of metrifonate-containing dressings on acute porcine wounds over a time course of 20 days. In addition, the procine studies were used to confirm treatment efficacy and appropriate doses that will guide future human Phase I/II clinical trials. Four weeks after diabetes induction, 24 square (15×15 mm) excisional full thickness wounds were created on the paravertebral and thoracic area with a distance of 2.5 cm of unwounded skin between each wound. The creation of 24 wounds (12 on each side) allows for comparison between various treatments and control treatment, which is usually the application of Tegaderm on the wound. Two different Metrifonate-Hydrogel doses were tested: a High dose-Met gel (HD met-gel) and Low dose (LD-Met gel) with blank gel (blk gel) serving as experimental control. The wound size was assessed at Days 3, 7, and 14 post-injury and all treatment were removed on Day 3.

### Results

The porcine studies revealed that metrifonate did not induce any adverse effects, and instead promoted better healing as evidenced by shorter wound lengths in histological assessments (FIGS. 31A & 31B). Quantification of wound closure at days 3, 7, and 14 post-injury revealed higher healing rates with both HD-Met gel and LD-Met gel, when compared to the blank gel (FIGS. 31C, 31D, and 31E).

### Example 27. Effects of Metrifonate in Tissue Cultures of Cells Involved in Wound Healing

### Method

Following the in silico prioritization of metrifonate as a key compound in promoting wound healing, we determined if addition of metrifonate influenced key cell functions in tissue cultures of cells that are implicated in wound healing, such as fibroblasts, keratinocytes and macrophages. The metabolic activity of fibroblasts was monitored with an Alamar Blue assay over three days following treatment with a range of metrifonate concentrations.

### Results

Alamar Blue assays of fibroblasts revealed that metrifonate concentrations over 10 μg/ml began to negatively affect cell viability (FIG. 32A). A metrifonate concentration of 10 μg/ml was thus selected for all subsequent experiments. In a wound healing scratch assay experiment, treatment of keratinocytes with metrifonate was found to significantly accelerate migration and facilitate wound closure after 24 hours (FIG. 32B). Furthermore, THP-1 derived macrophages polarized to MO, M1 and M2 phenotypes displayed a notable increase in gene expression of anti-inflammatory and pro-regenerative IL-10, by 30-fold in MO and 10-fold in M2 (FIG. 32C). In addition, M1 cells also displayed a remarkable downregulation of the pro-inflammatory cytokines TNF and IL-10, by 40-fold and 12-fold respectively (FIGS. 32D and 32E). Taken together, these findings indicate that metrifonate exerts multiple beneficial effects that promote integral processes in wound healing, such as expedited re-epithelialization and excessive inflammation attenuation.

### Example 28. Measurement of Metrifonate Release Rates from Hydrogel Bandages

### Method

Alginate is an FDA approved polysaccharide widely used to fabricate commercial wound dressings (e.g., ALGICELL®), and can be configured to provide a sustained (>7 days) release of different metrifonate concentrations after placement on wounds. Metrifonate was incorporated into alginate solutions used for fabricating the bandage, and the resulting solution was placed into pre-formed molds, frozen, and lyophilized to create micron-sized pores in the bandage as illustrated in the schematic shown in FIG. 33A. The cryogels were then cross-linked with calcium chloride, and re-lyophilized to form dry, off-the-shelf bandages that can be stored and appropriately sized to cover biopsy punch inflicted wounds. Further details regarding the fabrication of this wound dressing material can be found in U.S. Pat. Nos. 10,016,524 and 10,391,136, the entire contents of each of these patents being incorporated herein by reference. The shelf life of these bandages is in the order of months when kept frozen under a dry atmosphere. In order to ensure continuous and sustained release of metrifonate over seven days or longer, Laponite was mixed with clay nanodisks with the metrifonate/alginate solution (FIG. 33A) prior to bandage fabrication. Metrifonate associates with the laponite through electrostatic interactions and, as a result, its initial release is diminished and the duration of release extended. See for example, Koshy S T, Zhang D K Y, Grolman J M, Stafford A G, Mooney D J. Injectable nanocomposite cryogels for versatile protein drug delivery. Acta Biomater. 2018; 65:36-43. Epub 2017/11/13. doi: 10.1016/j.actbio.2017.11.024. PubMed PMID: 29128539; PMCID: PMC5716876, the entire contents of which is incorporated herein by reference.

Release of metrifonate from alginate hydrogels was measured using an acetylcholinesterase (AChE) activity assay kit from day 1 to day 7. Each metrifonate loaded hydrogel was first submerged in 500 microliter phosphate buffer saline (PBS). The supernatant from high dose (initial load: 2 mg/ml), medium dose (initial load: 0.5 mg/ml), and low dose (initial load 0.08 mg/ml) metrifonate hydrogels were collected for AChE activity detection from day 1 to day 7.

### Results

Measurements of the AChE inhibitor concentration using the AChE assay kit showed that the cumulative release from high dose metrifonate hydrogel (HD Met-gel) and medium dose metrifonate hydrogel (MD Met-gel) reached equivalent points at day 3, with concentrations of 1000 μg/ml and 250 μg/ml, respectively (FIG. 33C). For low dose metrifonate hydrogel (LD Met-gel), daily metrifonate release was determined in micrograms, to better illustrate the dynamics of drug release (FIG. 33C).

### Example 29. Effects of Metrifonate Releasing Bandages on Wound Healing In Vivo

### Methods

The in vivo effects of metrifonate releasing bandages were evaluated in db/db mice, an established model of impaired diabetic wound healing. Since the release of metrifonate was seen to reach an equivalent point at day 3 (D3) in the experiments described above, metrifonate hydrogels were removed or replaced every 3 days post-wounding. Control groups in this study were Tegaderm and topical metrifonate solution (Met-sol).

### Results

Of the different treatment groups, low dose (LD) Met-gel removed at Day 3 was seen to be the most efficient in accelerating mouse dorsal wounds healing and initiating the re-epithelialization process. These results are evident from the images shown in FIG. 34A, and time course of wound closure measurements shown FIG. 34B. Quantification of wound sizes on Day 10 revealed that LD Met-gel removed at Day 6, as well as MD Met-gel removed at Day 3 improved wound healing to a lesser degree, while application of soluble metrifonate (TTMet-sol) showed no significant effect compared to Tegaderm application. These results indicated that application of low doses of metrifonate during the early stages of wound healing, coinciding with the inflammatory phase, improve impaired diabetic wound healing.

### Example 30. Analysis of Effects of Metrifonate on Cell-Cell Interactions During Wound Healing

To analyze the effect of metrifonate on cell-cell interactions during wound healing further methods in addition to multispectral immunofluorescence can be used. To this end, Multiplexed error-robust fluorescence in situ hybridization (MERFISH) can be used enabling quantification of the expression of up to 500 genes in a tissue section at single cell and single molecule resolution. The MERFISH workflow builds upon the highly standardized and optimized implementation on the MERSCOPE instrument (Vizgen, MA). This production-grade pipeline addresses many major hurdles of the classic MERFISH assay, including stitching, registration, and segmentation of image data. The commercial MERSCOPE instrument enables analysis of a sectioned tissue block that is placed on the MERSCOPE slide (Vizgen) and permeabilized overnight, following 5 mL 70% ethanol addition. For autofluorescence quenching, the slide is placed in the MERSCOPE Photobleacher (Vizgen) for 3 hours. Formamide wash buffer is added and incubated at 37° C. for 30 minutes. 50 μl MERSCOPE Gene Panel Mix is added to the section and incubated at 37° C. for 40 hours. 50 μl Gel Slick Solution is placed onto the Gel Coverslip and 5 μl of Gel Embedding Solution is added and following 1 minute, 50 μl of the Gel Embedding Solution is placed on the tissue section and incubated at room temperature for 1.5 hours. The Vizgen Clearing Premix and Proteinase K are used to clear the tissue, while keeping RNA/DNA intact, by following the human liver-specific protocol. Samples are incubated for 24 hours or until the tissue section becomes transparent, and then are incubated with DAPI and PolyT Staining Reagent for 15 minutes. Slides are placed into the MERSCOPE and scanned. Up to 1 cm² will be analyzed using a probe set capturing up to 500 targets. Note that 118 targets have been selected by reanalyzing the internal single cell data for wound healing in human and mouse. Additional targets can be incorporated into the assay with the bulk and single cell studies.

MERSCOPE files are converted to Zarr-AnnData and OME-TIFF format. Extensive QC metrics are extracted (FIG. 35), including the number of cells and RNA molecules detected, sample dimensions, RNA molecule density, and statistics per probe to enable identification of low-performing probes. The multiplexed immunofluorescence assays from serial sections can be overlayed using non-rigid slide registration. MERSCOPE and overlayed multiplexed IF datasets can be further explored with Giotto (see Dries et al., “Giotto: a toolbox for integrative analysis and visualization of spatial expression data”, Genome Biology: 2021; 22(1):78.doi:10.1186/s13059-021-02286-2, the entire contents thereof being incorporated herein by reference) to perform differential expression analyses, cell-cell colocalization and communication, as well as to identify spatially variable genes. Spatially distinct regions can be detected and scRNAseq data can be integrated and registered on the spatial assays using Tangram. The MERSCOPE datasets will be utilized to identify the effects of metrifonate on the tissue microenvironment in situ and to maximize our ability to interrogate tissue and to investigate findings from the bulk, proteomic, and single cell studies.

Experience has shown that the effect of size in pig studies was similar, if not better, than mice studies. The same can apply for this study and, as a result, nine wounds per treatment can be sufficient to provide more than 80% power at 5% level of significance. Across all modalities (single cell, spatial), 4+ biological replicates can be utilized per group/time point enabling us to achieve high granularity and power in all comparisons. There can be more than 99% power to detect rare cellular populations (less than 1% of cells) with an adequate number of cells per cluster (>100) for at least 10 rare cell populations per group.

Comparisons between groups can be performed using the ANOVA test for normally distributed data and the Kruskal-Wallis test for data that are not distributed normally. The Fit General Linear Model can be employed for repeated measurement analysis. RNA-seq data can be processed with data being quality-checked using FastQC55 and reads can be aligned to reference genome STAR and a 2-pass approach and quantifying reads with featureCounts(72). Differential gene expression analysis can be performed using DESeq2(55), while ClusterProfiler (v3.12.0)(73) can be utilized for downstream functional investigations.

Other complementary assays, including single nucleus sequencing (in lieu of single cell sequencing) as well as different additional spatial tissue profiling platforms such as 10× Genomics Visium, Akoya CODEX, NanoString GeoMx DSP, and others, which can be utilized to substitute or complement the above assays.

### Example 31. Effect of Metrifonate on Wound Healing in Diabetic Humanized Mice

### Rationale

Human skin transplantation on immunodeficient mice, which support engraftment, are a valuable study model for investigating mechanisms involved in wound healing. It has been demonstrated in various studies that, after injury, the regeneration of human skin transplanted onto nude mice mimics the process observed in injured patient skin; thus making the establishment of a humanized diabetic wound healing model a valuable resource for studying the wound healing process in vivo. Human skin grafted onto the nude mouse has previously been shown to be able to regenerate after an excisional wound is made through the entire thickness of the graft.

### Method

To generate a humanized skin graft mice model, Foxn1nu nude male mice of 6 weeks old can be used as the recipient animals. These mice are devoid of T cells and suffer from a lack of cell-mediated immunity making them a model of choice for xenograft studies. Mice dorsal skin will be excised with an area where a graft of an equal size of full-thickness or split-thickness human skin will be sutured. Human skin is obtained from IRB-approved discarded human skin from plastic surgeries. Grafted mice are left to heal and accommodate the new tissue for 5 weeks, where the graft fully matures. Diabetes is induced in stable human skin engrafted mice 5 weeks post-grafting via intraperitoneal injections with streptozotocin (STZ). Mice sustain a hyperglycemic state for 7 weeks prior to wounding experiments. Following the sustained diabetic state period, wounds are inflicted on the transplanted human skin with a 3 mm punch biopsy, treatment options include the best two Metrifonate-Hydrogel dressings, previously determined by the mice and pig studies for the preferred two doses. These dressings are applied to each individual wound, as well as a blank hydrogel formulation. As bandages can provide sustained release for at least three days, changes can be initially performed every three days, and the potential of weekly changes will be explored with the chosen two best doses. At days 3, 5 and 10 post-wounding and dressing treatment, mice are euthanized and wound tissue can be harvested for histological and molecular analysis. Wound healing can be monitored using standardized photography daily during the course of 3, 5 and 10 days. Wound area can be presented as % of original wound. Histological and immunohistochemistry studies are performed as described for the mice studies.

### Results

Measurements of wound closure in diabetic humanized mice demonstrated improved wound healing with metrifonate, compared to blank gels, to a degree that was similar, if not better, than wound healing observed in db/db mice. These results are shown in FIG. 36A.

### Example 32. Effect of Metrifonate on Gene Expression in Diabetic Humanized Mice

RT-qPCR experiments were performed upon sacrifice of humanized mice described above, to analyze gene expression. These results showed that metrifonate application on the wound increased the expression of Il10 (FIG. 37A), Arg (FIG. 37B), and the human gene VEGFA (FIG. 37C). These genes are well known to improve impaired diabetic wound healing, see Berry-Kilgour et al., Advancements in the Delivery of Growth Factors and Cytokines for the Treatment of Cutaneous Wound Indications; Adv Wound Care (New Rochelle) 2021; 10(11):596-622. Epub 20201125. doi:10/1089?wound.2020.1183. PubMed PMID:33086946: PMCID: PMC8392095.

### Example 33. AlamarBlue™ Assay

In a further example, an alamarBlue™ Assay can be used as follows: Human dermal fibroblasts (immortalized cell line, abm Cat. No. T0904) are cultured in complete medium (PriGrow III with 10% Fetal Bovine Serum, 100U/L penicillin, and 100 ug/L streptomycin) at 37° C. in 5% CO2. The cells are passaged every 7 days using 0.025% trypsin-ethylenediaminetetraacetic acid (Life Technologies) and plated at 10,000 cells per well in 96-well plates. Cells are incubated in complete medium with metrifonate or other AchE inhibitor concentrations: 0.05, 0.1, 1, 5, 10, 20 and 40 μg/ml and positive control and negative control are generated by culturing cells without metrifonate and wells with no cells seeded. Cell viability is determined by alarmarBlue™ (Invitrogen) after 48h, and 72h of incubation. Briefly, after 48 or 72h of incubation the media containing inhibitors is removed and replaced with 10% v/v alamarBlue™ in complete media and the plate is incubated at 37° C. for further 24 h. After this incubation period, plates are read at an excitation range of 540-570 nm and an emission range of 580-610 nm. All AchE inhibitors are expected to not negatively affect cell proliferation up to the concentration of 10 μg/ml.

### Example 34. Scratch Assay

Keratinocyte HaCaT cells (ATCC) are cultured in complete medium (Dulbecco's Modified Eagle Medium with 10% Fetal Bovine Serum, 100U/L penicillin, and 100 ug/L streptomycin) at 37° C. in 5% CO2. The cells are passaged every 5 days using 0.025% trypsin-ethylenediaminetetraacetic acid (Life Technologies) detachment and plated at 100,000 cells per well in 24-well plates. The cell monolayer is scratched in a straight line using a P200 pipette tip. Debris are removed by washing once with complete medium, then cells were incubated in serum free medium with metrifonate or other AchE inhibitor (with concentrations ranging from 0.05 to 40 μg/ml) throughout the experiment. Images are taken immediately after the scratch (0 h), in 6 h, 12 h, 24 h, 48 h, and 72 h for at least four wells per condition. Scratch areas are analyzed using ImageJ/FIJI. At least three independent experiments are performed. All AchE inhibitors are expected to promote faster wound coverage up to the concentration of 10 μg/ml.

### Example 35. Wound Healing In Vivo

Two 6 mm diameter full-thickness skin wounds are created on 16-week old db/db mice and alginate dressings with metrifonate or other AchE inhibitor (and cumulative release dose determined from the highest dose that does not cause reduction in proliferation in vitro—we hypothesize similar dose to metrifonate) are placed immediately after injury on the wounds. Control includes blank dressings. The dressings are removed on day 3 after wounding and wound closure is measured on days 3, 5, 7 and 10, when the experiment is completed. Wound tissue is collected and processed for histology, RNA and protein extraction. All AchE inhibitors are expected to accelerate wound healing compared to the blank dressings.

1. A wound dressing comprising:
an alginate hydrogel having a therapeutic amount of an acetylcholinesterase inhibitor distributed therein for release onto an ulcerative wound during a delivery period.

2. The wound dressing of claim 1, wherein the wound dressing comprises an absorbent material.

3. The wound dressing of claim 1, wherein the acetylcholinesterase inhibitor comprises metrifonate.

4. The wound dressing of claim 3, wherein the metrifonate is electrostatically bound within the alginate hydrogel until release onto the wound.

5. The wound dressing of claim 4, further comprising an agent that electrostatically inhibits release of the metrifonate during the delivery period.

6. The wound dressing of claim 5, wherein the agent comprises laponite.

7. The wound dressing of claim 3, wherein the metrifonate is encapsulated in polymer microspheres dispersed within the alginate hydrogel.

8. The wound dressing of claim 7, wherein the polymer microspheres comprise poly lactide-co-glycolide (PLG).

9. The wound dressing of claim 2, wherein the wound dressing comprises the metrifonate in an amount sufficient to result in an accumulated concentration of metrifonate in the wound below 1000 micrograms/ml.

10. The wound dressing of claim 1, wherein the acetylcholinesterase inhibitor activates cholinergic receptor muscarinic 1 (Chrm1).

11. The wound dressing of claim 1, wherein the wound dressing comprises the acetylcholinesterase inhibitor in an amount sufficient to:
(a) increase the wound closure rate relative to an ulcerative wound that is not treated with the acetylcholinesterase inhibitor;
(b) increase one or more of re-epithelialization of the wound and blood vessel density of the wound relative to an ulcerative wound that is not treated with the acetylcholinesterase inhibitor;
(c) increase one or more of total mast cells in the wound and degranulated mast cells in the wound relative to an ulcerative wound that is not treated with the acetylcholinesterase inhibitor;
(d) decrease expression of one or more genes selected from the group consisting of Transforming growth factor beta 1 (TGFB1), alpha-2 actin (ACTA2), interleukin 1B, (IL-1B), Matrix metalloproteinase 9 (MMP9) and Neutrophil gelatinase-associated lipocalin (NGAL), relative to an ulcerative wound that is not treated with the acetylcholinesterase inhibitor;
(e) decrease the levels of neutrophil marker NIMP-R14+ cells in the wound and/or increase the number of macrophage marker F4/80+ cells in the wound, relative to an ulcerative wound that is not treated with the wound dressing comprising the acetylcholinesterase inhibitor; and/or
(f) increase expression of one or more genes selected from the group consisting of cholinergic receptor muscarinic 1 (Chrm1), Chrm2, Chrm3, and cholinergic receptor nicotinic alpha (Chrna7), relative to an ulcerative wound that is not treated with the wound dressing comprising the acetylcholinesterase inhibitor.

12-16. (canceled)

17. The wound dressing of claim 1, wherein the acetylcholinesterase inhibitor is selected from the group consisting of donepezil, revastigmine, galantamine and tacrine.

18. The wound dressing of claim 1, wherein the wound dressing is formulated to be applied to the ulcerative wound during an inflammatory phase of the ulcerative wound.

19. The wound dressing of claim 1, wherein the wound dressing further comprises an adhesive layer to attach the wound dressing to skin surrounding the ulcerative wound on a foot of a subject.

20. A combination comprising the wound dressing of claim 1, and a further wound dressing.

21. The combination of claim 20, wherein the further wound dressing comprises a recombinant RNA molecule encoding one or more polypeptides selected from the group consisting of chitinase-3-like protein 1(CHI3L1), fibroblast growth factor 2 (FGF-2), interleukin-2 ORF7 (IL-2 ORF7), interleukin (IL-2) and interleukin-17A (IL-17A).

22-28. (canceled)

29. A method of treating a diabetic ulcer wound in a subject, comprising:
applying a wound dressing to the diabetic ulcer wound of the subject, the wound dressing comprising:
a) a therapeutically effective amount of an acetylcholinesterase inhibitor; and
b) an alginate hydrogel configured to release a therapeutic dose of the acetylcholinesterase inhibitor onto the wound for a delivery period.

30. The method of claim 29, wherein the acetylcholinesterase inhibitor comprises metrifonate ((2,2,2-trichloro-1-hydroxyethyl) dimethylphosphonate).

31. The method of claim 29, wherein the alginate hydrogel includes an agent that electrostatically inhibits release of metrifonate during the delivery period.

32. The method of claim 31, wherein the agent comprises laponite.

33. The method of claim 30, wherein the metrifonate is encapsulated in a plurality of polymer microspheres.

34. The method of claim 33, wherein the polymer microspheres comprise poly lactide-co-glycolide (PLG).

35. The method of claim 30, wherein the acetylcholine esterase inhibitor is applied to the diabetic ulcer wound in an amount sufficient to result in an accumulated concentration of metrifonate in the wound below 1000 micrograms/ml.

36. The method of claim 29, wherein the acetylcholinesterase inhibitor activates cholinergic receptor muscarinic 1 (Chrm1).

37. The method of claim 29, wherein the acetylcholinesterase inhibitor is selected from the group consisting of donepezil, rivastigmine, gelantamine and tacrine.

38. The method of claim 29, wherein the wound dressing further comprises an adhesive layer to attach the wound dressing to skin surrounding the diabetic wound on a foot of the subject.

39. The method of claim 29, wherein the acetylcholinesterase inhibitor is applied to the diabetic ulcer wound in an amount sufficient to:
(a) increase the wound closure rate relative to a diabetic ulcer wound that is not treated with the acetylcholinesterase inhibitor;
(b) increase one or more of re-epithelialization of the wound and blood vessel density of the wound relative to a diabetic ulcer wound that is not treated with the acetylcholinesterase inhibitor;
(c) increase one or more of total mast cells in the wound and degranulated mast cells in the wound relative to a diabetic ulcer wound that is not treated with the acetylcholinesterase inhibitor;
(d) decrease expression of one or more genes selected from the group consisting of Transforming growth factor beta 1 (TGFB1), alpha-2 actin (ACTA2), interleukin 1B, (IL-1B), Matrix metalloproteinase 9 (MMP9) and Neutrophil gelatinase-associated lipocalin (NGAL), relative to a diabetic ulcer wound that is not treated with the acetylcholinesterase inhibitor;
(e) decrease the levels of neutrophil marker NIMP-R14+ cells in the wound and/or increase the number of macrophage marker F4/80+ cells in the wound, relative to a wound that is not treated with the wound dressing comprising the acetylcholinesterase inhibitor; and/or
(f) increase expression of one or more genes selected from the group consisting of cholinergic receptor muscarinic 1 (Chrm1), Chrm2, Chrm3, and cholinergic receptor nicotinic alpha (Chrna7), relative to a wound that is not treated with the wound dressing comprising the acetylcholinesterase inhibitor.

40-44. (canceled)

45. The method of claim 29, wherein the wound dressing in applied to the diabetic ulcer wound during an inflammatory phase of the wound.

46. The method of claim 45, further comprising applying a further wound dressing to the wound after the inflammatory phase.

47. The method of claim 46, wherein the further wound dressing comprises a recombinant RNA molecule encoding one or more polypeptides selected from the group consisting of chitinase-3-like protein 1(CHI3L1), fibroblast growth factor 2 (FGF-2), interleukin-2 ORF7 (IL-2 ORF7), interleukin (IL-2) and interleukin-17A (IL-17A).

48-54. (canceled)

55. The method of claim 46, wherein the further wound dressing is applied during the proliferation phase of healing of the wound.


SEQUENCE LISTING (14 sequences)

SEQ ID NO: 1 - DNA, 1152 bp, Homo sapiens
  misc_feature 1..1152: note = Human wildtype chitinase-3-like protein 1 (CHI3L1) nucleic acid sequence
  source 1..1152: mol_type = genomic DNA
atgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgtggccaggatctgcgcttccctctcaccaatgccatcaaggatgcactcgctgcaacgtag

SEQ ID NO: 2 - protein, 383 aa, Homo sapiens
  REGION 1..383: note = misc_feature - Human wildtype chitinase-3-like protein 1 (CHI3L1) amino acid sequence
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 3 - DNA, 468 bp, Homo sapiens
  misc_feature 1..468: note = Human wildtype fibroblast growth factor 2 (FGF-2) nucleic acid sequence
  source 1..468: mol_type = genomic DNA
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 4 - protein, 155 aa, Homo sapiens [sequence omitted]

SEQ ID NO: 5 - DNA, 93 bp, Homo sapiens
  misc_feature 1..93: note = Human wildtype interleukin-2 ORF7 (IL-2 ORF7) nucleic acid sequence
  source 1..93: mol_type = genomic DNA
atgcaatttatactgttaattctggaaaaatattatgggggtgtcaaaatgttttacatattacacatattttcaaagactttacctgtctga

SEQ ID NO: 6 - protein, 30 aa, Homo sapiens
  REGION 1..30: note = misc_feature - Human wildtype interleukin-2 ORF7 (IL-2 ORF7 ) amino acid sequence
MQFILLILEKYYGGVKMFYILHIFSKTLPV

SEQ ID NO: 7 - DNA, 468 bp, Homo sapiens
  misc_feature 1..468: note = Human wildtype interleukin-17A (IL-17A) nucleic acid sequence
  source 1..468: mol_type = genomic DNA
atgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaa

SEQ ID NO: 8 - protein, 155 aa, Homo sapiens
  REGION 1..155: note = misc_feature - Human wildtype interleukin-17A (IL-17A) amino acid sequence
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 9 - DNA, 477 bp, Mus musculus
  misc_feature 1..477: note = Mouse wildtype interleukin-17A (IL-17A) nucleic acid sequence
  source 1..477: mol_type = genomic DNA
atgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaa

SEQ ID NO: 10 - protein, 158 aa, Mus musculus
  REGION 1..158: note = misc_feature - Mouse wildtype interleukin-17A (IL-17A) amino acid sequence
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 11 - DNA, 462 bp, Homo sapiens
  misc_feature 1..462: note = IL-2 nucleic acid sequence
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttga

SEQ ID NO: 12 - protein, 153 aa, Homo sapiens
  REGION 1..153: note = misc_feature - IL-2 amino acid sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - DNA, 510 bp, Mus musculus
  misc_feature 1..510: note = IL-2 nucleic acid sequence
  source 1..510: mol_type = genomic DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 14 - protein, 169 aa, Mus musculus
  REGION 1..169: note = misc_feature - IL-2 amino acid sequence
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ